(19) United States
(12) Patent Application Publication
(10) Pub. No.: US 2020/0385785 A1
(43) Pub. Date: Dec. 10, 2020

(54) COMPOSITIONS AND METHODS FOR BARCODING

(71) Applicant: Rootpath Genomics, Inc., Watertown, MA (US)

(72) Inventors: Xi Chen, Newton, MA (US); Ely Porter, Medford, MA (US)

(21) Appl. No.: 16/905,270

(22) Filed: Jun. 18, 2020

Related U.S. Application Data
(63) Continuation of application No. PCT/US18/66748, filed on Dec. 20, 2018.
(60) Provisional application No. 62/609,756, filed on Dec. 22, 2017.

(51) Int. Cl. C12Q 1/6806

(52) U.S. Cl. CPC C12Q 1/6806

(57) ABSTRACT

Provided herein are compositions and methods to entrap a target in a hardened particle (e.g., a hydrogel particle) and barcode the target for downstream applications. The target provided herein can be a polynucleotide, a polypeptide, or an organelle from a single cell, a single complex of cell or a single exosome. The present disclosure provides various strategies to immobilize or restrict diffusion of the target within a hardened particle so that the target can be stably entrapped or embedded. A split-and-pool combinatorial strategy described herein can be used to barcode each hardened particle so that one or more targets from the same hardened particle can be barcoded with a common barcode sequence. The barcoded sequences can further be analyzed in a downstream application, for example, sequencing.

### CROSS-REFERENCE

This application is a continuation of International Patent Application No. PCT/US18/66748, filed on Dec. 20, 2018, which claims the benefit of U.S. Provisional Patent Application No. 62/609,756, filed Dec. 22, 2017, the entire contents of each of which is entirely incorporated herein by reference.

### SEQUENCE LISTING

The instant application contains a Sequence Listing which has been submitted electronically in ASCII format and is hereby incorporated by reference in its entirety. Said ASCII copy, created on Jun. 16, 2020, is named 53563-701 301 SL.txt and is 4,669 bytes in size.

### BACKGROUND OF THE INVENTION

Many high-throughput single-cell analysis methods have emerged over the last few years. Droplet-based single cell RNA-Seq methods such as DropSeq, 10×GEM, and inDrop are some of the examples.

### SUMMARY OF THE INVENTION

There can be some limitations for droplet-based method. For example, not every droplet has a bead leading to loss of cells since the mRNA from these cells cannot be barcoded. Also, the types of manipulation that can be performed to the target molecules may be fairly limited since it is non-trivial to deliver reagent to and remove component from the droplets. Therefore, it has been challenging to extend the success of single-cell RNA-Seq to other types of analysis such as protein analysis and histone modification analysis at single cell level with high throughput. Recognized herein is a need for high-throughput strategies for sample preparation and efficient barcoding for various down-stream analyses.

According to an aspect of the present disclosure, provided herein is a method of barcoding comprising contacting a barcode to a hardened particle comprising polymerized or gelled polymers and/or monomers and a single cell, a single complex of cell, a single exosome, a target or derivative thereof, or a combination thereof; and diffusing the barcode into the hardened particle. In some cases, the method further comprises forming a compartment. In some cases, the method comprises forming the compartment before contacting the barcode. In some cases, the compartment comprises a plurality of polymerizable or gellable polymers and/or monomers; the single cell, the single complex of cells, the single exosome, the target, or a combination thereof; and an immobilizable probe. In some cases, the method further comprises polymerizing or gelling the plurality of polymers and/or monomers in the compartment, thereby forming the hardened particle. In some cases, the polymers comprise polysaccharides, polyacrylamides, polyacrylic acids, polyethylene glycols, polyvinyl alcohols, polymethacrylamides, or any combination thereof. In some cases, the polysaccharides comprise agarose, hyaluronic acids, carboxymethycellose, chitosan, alginate, or any combination thereof. In some cases, the monomers comprise acrylic acids, acrylamides, methacrylamides, methacrylic acids, or any combination thereof. In some cases, the compartment or the hardened particle further comprises a crosslinking agent. In some cases, the compartment or the hardened particle further comprises a polymerization initiator. In some cases, the polymerization initiator is a photo-initiator. In some cases, the polymerization initiator is ammonium persulfate (APS), N,N,N′,N′-tetramethylethane-1,2-diamine (TEMED), Lithium- and magnesium phenyl-2,4,6-trimethylbenzoylphosphinates (TMPPL and TMPPM), sodium 4-[2-(4-morpholino)benzoyl-2-dimethylamino]-butylbenzenesulfonate (MBS), methylated-O-cyclodextrin (MβCD), or 2,2-dimethoxy-2-phenyl acetophenone (DMPA), or any combination thereof. In some cases, the method further comprises removing a thin liquid layer surrounding the hardened particle. In some cases, the single cell, the single complex of cells, or the single exosome is lysed. In some cases, the method further comprises lysing the single cell, the single complex of cells, or the single exosome before or after polymerizing or gelling the plurality of polymers and/or monomers. In some cases, the target or derivative thereof is from the single cell, the single complex of cells, or the single exosome. In some cases, the target or derivative thereof is bound to the immobilizable probe. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the modified DNA is a methylated DNA. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the target is a target polynucleotide and the derivative of the target is a complementary strand of the target polynucleotide. In some cases, the complementary strand is a cDNA strand or a templated-switched cDNA strand. In some cases, the target polypeptide is a peptide, a protein or a portion thereof, a modified protein or a portion thereof, a secreted protein, a protein complex, or any combination thereof. In some cases, the immobilizable probe comprises a targeting moiety, which targeting moiety links the immobilizable probe to the target or derivative thereof. In some cases, the targeting moiety is a polynucleotide, a polypeptide, or a chemical group. In some cases, the target is a target polynucleotide and the targeting moiety is a primer. In some cases, the primer is a reverse transcription primer. In some cases, the reverse transcription primer comprises a poly-deoxy-thymidine nucleotide sequence. In some cases, the method further comprises annealing the primer to the target polynucleotide. In some cases, the method further comprises extending the primer to generate a complementary strand of the target polynucleotide. In some cases, extending is performed before or after the polymerizing or gelling of the polymers and/or monomers. In some cases, the polypeptide is an antibody or a fragment thereof, or a peptide aptamer. In some cases, the chemical group forms a covalent bond with an amino acid side chain or a nucleobase of the target. In some cases, the chemical group is an N-hydroxysuccinimide (NHS) ester, a maleimide group, a hydrazide group, an azide group, or Label-IT linker and reactive group. In some cases, the amino acid side chain comprises an amine group, a carboxylate group, a carbonyl group, or a sulfhydryl group. In some cases, the nucleobase is guanine, adenine, cytosine or thymine. In some cases, the immobilizable probe comprises an immobilization moiety, which immobilization moiety links the immobilizable probe to one or more of the polymers and/or monomers of the polymerized or gelled plurality. In some cases, the immobilization moiety wherein the immobilization moiety is a chemical that is co-polymerized with the polymers and/or monomers, a chemical or protein that stably interact with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the method further comprises attaching the barcode to a barcode-accepting polynucleotide, the barcode-accepting polynucleotide is the immobilizable probe or the target or derivative thereof. In some cases, the compartment or the hardened particle further comprises a detection probe, which detection probe binds to the target or derivative thereof. In some cases, the barcode is attached to the detection probe. In some cases, the barcode has a size that is smaller than a pore size of the hardened particle. In some cases, the barcode is from 5 to 500 nucleotides in length. In some cases, the hardened particle is a plurality of hardened particles. In some cases, attaching the barcode within the plurality of hardened particles comprises: pooling the plurality of hardened particles; dividing the plurality of hardened particles into separate first populations; attaching a first oligonucleotide comprising a first barcode sequence segment to the separate first populations, wherein each separate first population comprises a different first barcode sequence segment; pooling the separate first populations to provide a first pooled population; separating the first pooled population into a plurality of second populations; attaching a second oligonucleotide comprising a second barcode sequence segment to the first oligonucleotide already attached to the second populations, wherein each of the plurality of second populations comprises a different second barcode sequence segment; and pooling the plurality of second populations to provide a second pooled population comprising a barcode library. In some cases, the first barcode sequence segment or the second barcode sequence segment is at least 1 nucleotide in length. In some cases, the first barcode sequence segment or the second barcode sequence segment is at least 4 nucleotides in length. In some cases, the first barcode sequence segment or the second barcode sequence segment is from about 4 to about 20 nucleotides in length. In some cases, the method further comprises washing the hardened particle.

According to another aspect of the present disclosure, provided herein is a method of barcoding, comprising: generating a population of vessels comprising a first total number of vessels, each vessel of the population of vessels comprising (i) a target or derivative thereof from a single cell, a single complex of cells, or a single exosome; and (ii) a plurality of polymerizable or gellable polymers and/or monomers; polymerizing or gelling the plurality of polymerizable or gellable polymers and/or monomers of the population of vessels, thereby forming a population of hardened particles; and contacting a barcode to the population of hardened particles, thereby forming a population of barcoded hardened particles comprising a second total number of barcoded hardened particles, each barcoded hardened particle of the population of barcoded hardened particles comprising (i) the target or derivative thereof from the single cell, the single complex of cells, or the single exosome; and (ii) polymerized or gelled polymers/monomers; wherein the second total number is at least 90%, 91%, 92%, 93%, 94%, 95%, 96%, 97%, 98%, 99%, or at least 100% of the first total number. In some cases, the barcode diffuses into the hardened particles. In some cases, the method further comprises removing a thin liquid layer surrounding the population of vessels. In some cases, the vessel or the hardened particle further comprises a non-cellular polynucleotide.

According to another aspect of the present disclosure, provided herein is a composition comprising a plurality of vessels, each vessel of the plurality comprising a hydrogel particle or a bead; an immobilizable probe embedded within the hydrogel particle or bead, wherein the immobilizable probe is immobilized to a diffusion restricting agent; and a target or derivative thereof embedded within the hydrogel particle or bead and linked to the immobilizable probe, wherein the target is from a single cell, a single complex of cells, or a single exosome, or the derivative thereof is a derivative of a target from a single cell, a single complex of cells, or a single exosome; wherein the hydrogel particle or the bead has a volume that is at least 50% of a volume of the vessel. In some cases, the target is from a different single cell, a different single complex of cells, or a different single exosome. In some cases, the hydrogel particle or the bead has a volume that is at least 60%, 70%, 80%, or 90% of the volume of the vessel. In some cases, the hydrogel particle or bead comprises a polymerized or gelled plurality of polymers and/or monomers. In some cases, the polymers comprise polysaccharides, polyacrylamides, polyacrylic acids, polyethylene glycols, polyvinyl alcohols, polymethacrylamides, or any combination thereof. In some cases, the polysaccharides comprise agarose, hyaluronic acids, carboxymethycellose, chitosan, alginate, or any combination thereof. In some cases, the monomers comprise acrylic acids, acrylamides, methacrylamides, methacrylic acids, or any combination thereof. In some cases, the immobilizable probe and the target or derivative thereof are linked by a covalent bond or a non-covalent interaction. In some cases, the immobilizable probe comprises an immobilization moiety, which immobilization moiety links the immobilizable probe to one or more polymers and/or monomers of the polymerized or gelled plurality. In some cases, the immobilization moiety is a chemical that is co-polymerized with the polymers and/or monomers, a chemical or protein that stably interacts with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the diffusion restricting agent is one or more polymers and/or monomers of the polymerized or gelled plurality. In some cases, the diffusion restricting agent is a polymer chain conjugated on the immobilizable probe. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the target is a target polynucleotide and the derivative of the target is a complementary strand of the target polynucleotide. In some cases, the complementary strand is a cDNA strand or a template-switched cDNA strand. In some cases, the target polypeptide is a peptide, a protein or a portion thereof, a modified protein or a portion thereof, a secreted protein, a protein complex, or any combination thereof. In some cases, the immobilizable probe further comprises a targeting moiety, which targeting moiety links the immobilizable probe to the target or derivative thereof. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotides sequence. In some cases, the polypeptide is an antibody or a fragment thereof, or a peptide aptamer. In some cases, the chemical group forms a covalent bond with an amino acid side chain or a nucleobase of the target. In some cases, the chemical group is an N-hydroxysuccinimide (NHS) ester, a maleimide group, a hydrazide group, an azide group or Label-IT linker and reactive group. In some cases, the amino acid side chain comprises an amine group, a carboxylate group, a carbonyl group, or a sulfhydryl group. In some cases, the nucleobase is guanine, adenine, cytosine or thymine. In some cases, each vessel of the plurality further comprises a lysing agent. In some cases, the lysing agent is a cell lysing agent. In some cases, the different single cell, the different single complex of cells, or the different single exosome is lysed. In some cases, the derivative is a copied product of the target. In some cases, the copied product of the target is linked to the immobilizable probe via a phosphodiester bond.

According to another aspect of the present disclosure, provided herein is a composition comprising a plurality of hydrogel particles or beads, each hydrogel particle or bead of the plurality comprising an immobilizable probe embedded within the hydrogel particle or the bead, wherein the immobilizable probe is immobilized to a diffusion restricting agent or has a hydrodynamic radius that is larger than a pore size of the hydrogel particle or the bead; a target or derivative thereof embedded within the hydrogel particle or the bead and linked to the immobilizable probe, wherein the target is from a single cell, a single complex of cells, or a single exosome or the derivative thereof is a derivative of a target from a single cell, a single complex of cells, or a single exosome; and a barcode. In some cases, each of hydrogel particle or bead comprises a polymerized or gelled plurality of polymers and/or monomers. In some cases, the diffusion restricting agent is one or more polymers and/or monomers of the polymerized or gelled plurality. In some cases, the diffusion restricting agent is a polymer chain conjugated on the immobilizable probe. In some cases, the polymer chain is a polyethylene glycol molecule or a polyacrylamide molecule. In some cases, the immobilizable probe comprises an immobilization moiety, which immobilization moiety links the immobilizable probe to one or more polymers and/or monomers of the polymerized or gelled plurality. In some cases, the immobilization moiety is a chemical that is co-polymerized with the polymers and/or monomers, a chemical or protein that stably interacts with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the target is a target polynucleotide and the derivative of the target is a complementary strand of the target polynucleotide. In some cases, the complementary strand is a cDNA strand or a template-switched cDNA strand. In some cases, the target polypeptide is a peptide, a protein or a portion thereof, a modified protein or a portion thereof, a secreted protein, a protein complex, or any combination thereof. In some cases, the immobilizable probe further comprises a targeting moiety, which targeting moiety links the immobilizable probe to the target or derivative thereof. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotides sequence. In some cases, the polypeptide is an antibody or a fragment thereof, or a peptide aptamer. In some cases, the chemical group forms a covalent bond with an amino acid side chain or a nucleobase of the target. In some cases, the chemical group is an N-hydroxysuccinimide (NHS) ester, a maleimide group, a hydrazide group, an azide group or Label-IT linker and reactive group. In some cases, the amino acid side chain comprises an amine group, a carboxylate group, a carbonyl group, or a sulfhydryl group. In some cases, the nucleobase is guanine, adenine, cytosine or thymine. In some cases, the different single cell, the different single complex of cells, or the different single exosome is lysed. In some cases, the barcode is attached to the target or derivative thereof or the immobilizable probe. In some cases, the barcode is from 5 to 500 nucleotides in length.

According to another aspect of the present disclosure, provided herein is a hardened particle comprising a polymerized or gelled plurality of polymers and/or monomers; a single cell, a single complex of cells, or a single exosome; an immobilizable probe linked to one or more polymers and/or monomers of the polymerized or gelled plurality; and a barcode. In some cases, the hardened particle is a hydrogel particle. In some cases, the immobilizable probe comprises an immobilization moiety, which immobilization moiety links the immobilization probe to one or more polymers and/or monomers of the polymerized or gelled plurality. In some cases, the immobilization moiety is a chemical that is co-polymerized with the polymers and/or monomers, a chemical or protein that stably interacts with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group.

According to another aspect of the present disclosure, provided herein is a hardened particle comprising: a polymerized or gelled plurality of polymers and/or monomers; a single cell, a single complex of cells, or a single exosome; an immobilizable probe, wherein the immobilizable probe has a hydrodynamic radius that is larger than a pore size of the vessel; and a barcode. In some cases, the immobilizable probe comprises an immobilization moiety. In some cases, the immobilization moiety is conjugated to a polymer chain. In some cases, the immobilization polymer is a polyethylene glycol molecule. In some cases, the hardened particle further comprises a barcode. In some cases, the hardened particle further comprises a target from the single cell, the single complex of cell, or the single exosome. In some cases, the target is bound to the immobilizable probe. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the modified DNA is a methylated DNA. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the hardened particle further comprises a complementary strand of the target polynucleotide. In some cases, the complementary strand is a cDNA strand. In some cases, the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some cases, the target polypeptide is a peptide, a protein or a portion thereof, a modified protein or a portion thereof, a protein complex, or any combination thereof. In some cases, the immobilizable probe further comprises a targeting moiety. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotide sequence. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. In some cases, the reactive group is an N-hydroxysuccinimide (NHS) ester, a maleimide group, a hydrazide group, an azide group or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine, adenine, cytosine or thymine. In some cases, the barcode is linked to the immobilizable probe, the target, or a complementary strand of the target polynucleotide. In some cases, the hardened particle further comprises a detection probe. In some cases, the barcode is linked to the detection probe. In some cases, the barcode is from 5 to 500 nucleotides in length. In some cases, the polymers are polysaccharides or polyacrylamides. In some cases, the polysaccharides are poly-agarobioses, hyaluronic acids, carboxymethycellose, chitosan or alginate. In some cases, the monomers are acrylamide or methacrylamide monomers. In some cases, the polymerized or gelled plurality of polymers and/or monomers are cross-linked.

According to another aspect of the present disclosure, provided herein is a droplet, comprising: a single cell, a single complex of cells, or a single exosome; a plurality of polymerizable or gellable polymers and/or monomers; an immobilizable probe linked to one or more polymers and/or monomers of the plurality; and a non-cellular polynucleotide; wherein the non-cellular polynucleotide is linked to the immobilizable probe. In some cases, the single cell, the single complex of cells, or the single exosome is lysed. In some cases, the immobilizable probe comprises an immobilization moiety. In some cases, the immobilization moiety links the immobilization probe to one or more polymerizable or gellable polymers and/or monomers of the plurality. In some cases, the immobilization moiety is a chemical that is co-polymerized with the polymers and/or monomers, a chemical or protein that stably interacts with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the droplet further comprises a target from the single cell, the single complex of cell, or the single exosome. In some cases, the target is bound to the immobilizable probe. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the modified DNA is a methylated DNA. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the droplet further comprises a complementary strand of the target polynucleotide. In some cases, the complementary strand is a cDNA strand. In some cases, the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some cases, the target polypeptide is a peptide, a protein or a portion thereof, a modified protein or a portion thereof, a protein complex, or any combination thereof. In some cases, the immobilizable probe further comprises a targeting moiety. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotide sequence. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. The droplet of claim 184-185, wherein the reactive group is an N-hydroxysuccinimide (NHS) ester, a maleimide group, a hydrazide group, an azide group or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine, adenine, cytosine or thymine. In some cases, the droplet is a water-in-oil droplet.

According to another aspect of the present disclosure, provided herein is a method, comprising: generating a population of vessels, each comprising a plurality of polymerizable or gellable polymers or monomers, wherein a subpopulation of the population comprises (i) a target from a single cell, a single complex of cells or a single exosome, and (ii) a non-cellular polynucleotide; and polymerizing or gelling the plurality of polymerizable or gellable polymers or monomers of the population, wherein each vessel of the subpopulation comprises a hardened particle. In some cases, the method further comprises contacting a barcode to the hardened particle. In some cases, the barcode diffuses into the hardened particle. In some cases, the method further comprises removing a thin liquid layer surrounding the hardened particle. In some cases, the single cell, the single complex of cells, or the single exosome is lysed. In some cases, the target is from the single cell, the single complex of cells, or the single exosome. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the modified DNA is a methylated DNA. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the method further comprises a complementary strand of the target polynucleotide. In some cases, the complementary strand is a cDNA strand. In some cases, the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some cases, the target polypeptide is a peptide, a protein or a portion thereof, a modified protein or a portion thereof, a protein complex, or any combination thereof. In some cases, each of the vessels or hardened particles further comprises an immobilizable probe. In some cases, the immobilizable probe is linked to one or more of the plurality of polymerizable or gellable polymers or monomers. In some cases, the immobilizable probe comprises a targeting moiety. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotide sequence. In some cases, the method further comprises annealing the primer to the target polynucleotide. In some cases, the method further comprises extending the primer to generate a complementary strand of the target polynucleotide. In some cases, extending is performed before or after the polymerizing or gelling of the polymers and/or monomers. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. In some cases, the reactive group is an N-hydroxysuccinimide (NHS) ester, a maleimide group, a hydrazide group, an azide group, or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine, adenine, cytosine or thymine. In some cases, the immobilizable probe comprises an immobilization moiety. In some cases, the immobilization moiety links the immobilizable probe to one or more of the polymers and/or monomers of the polymerized or gelled plurality. In some cases, the immobilization moiety a chemical that is co-polymerized with the polymers and/or monomers, a chemical or protein that stably interact with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the barcode is attached to the immobilizable probe, the target, or the complementary strand of the target polynucleotide. In some cases, the non-cellular polynucleotide is a barcode-accepting polynucleotide. In some cases, the barcode is attached to the barcode-accepting polynucleotide. In some cases, the compartment or vessel further comprises a detection probe. In some cases, the barcode-accepting polynucleotide is attached to the detection probe. In some cases, the compartment or the vessel is a water-in-oil droplet. In some cases, the hardened particle is a hydrogel particle.

According to another aspect of the present disclosure, provided herein is a method, comprising: generating a population of vessels, each comprising a plurality of polymerizable or gellable polymers or monomers, wherein a subpopulation of the population comprises a single cell, a single complex of cells or a single exosome; polymerizing or gelling the plurality of polymerizable or gellable polymers or monomers of the population, wherein each vessel of the subpopulation comprises a hardened particle; and lysing the single cell, the single complex of cell, or the single exosome. In some cases, the method further comprises removing a thin liquid layer surrounding the hardened particle. In some cases, the method further comprises contacting a barcode to the hardened particle. In some cases, the barcode diffuses into the hardened particle. In some cases, the vessel or hardened particle further comprises a target from the single cell, the single complex of cells, or the single exosome. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the modified DNA is a methylated DNA. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the method further comprises a complementary strand of the target polynucleotide. In some cases, the complementary strand is a cDNA strand. In some cases, the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some cases, the target polypeptide is a peptide, a protein or a portion thereof, a modified protein or a portion thereof, a protein complex, or any combination thereof. In some cases, each of the vessels or hardened particles further comprises an immobilizable probe. In some cases, the immobilizable probe is linked to one or more of the plurality of polymerizable or gellable polymers or monomers. In some cases, the immobilizable probe comprises a targeting moiety. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotides sequence. In some cases, the method further comprises annealing the primer to the target polynucleotide. In some cases, the method further comprises extending the primer to generate a complementary strand of the target polynucleotide. In some cases, extending is performed before or after the polymerizing or gelling of the polymers and/or monomers. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. In some cases, the reactive group is an N-hydroxysuccinimide (NHS) ester, a maleimide group, a hydrazide group, an azide group, or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine, adenine, cytosine or thymine. In some cases, the immobilizable probe comprises an immobilization moiety. In some cases, the immobilization moiety links the immobilizable probe to one or more of the polymers and/or monomers of the polymerized or gelled plurality. In some cases, the immobilization moiety a chemical that is co-polymerized with the polymers and/or monomers, a chemical or protein that stably interact with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the barcode is attached to the immobilizable probe, the target, or the complementary strand of the target polynucleotide. In some cases, the compartment or vessel further comprises a detection probe. In some cases, the barcode is attached to the detection probe. In some cases, the method further comprises washing the hardened particles extensively. In some cases, the compartment or the vessel is a water-in-oil droplet. In some cases, the hardened particle is a hydrogel particle.

According to another aspect of the present disclosure, provided herein is a method of barcoding a target, comprising: forming a plurality of vessels each comprising: a plurality of polymerizable or gellable polymers and/or monomers; a target; polymerizing or gelling the polymers and/or monomers in each of the plurality of vessels to form a hardened particle, thereby forming a plurality of hardened particles; generating a barcode sequence in each of the plurality of hardened particles, thereby generating a barcoded target sequence. In some cases, each of the plurality of vessels further comprises a single cell, a single complex of cells, or a single exosome. In some cases, the target is from the single cell, the single complex of cells, or the single exosome. In some cases, the target is a target polynucleotide, a target polypeptide, or a target organelle. In some cases, the target polynucleotide is a target ribonucleic acid (RNA) or a target deoxyribonucleic acid (DNA). In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the target DNA is a genomic DNA, a modified DNA, or a combination thereof. In some cases, the modified DNA is a methylated DNA. In some cases, the target organelle comprises acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some cases, the target polypeptide is a peptide, a protein or a portion thereof, a modified protein or a portion thereof, a protein complex, or any combination thereof. In some cases, each of the plurality of vessels further comprises an immobilizable probe linked to one or more polymerizable or gellable polymers or monomers of the plurality. In some cases, the immobilizable probe comprises a targeting moiety. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotides sequence. In some cases, the method further comprises annealing the primer to the target polynucleotide. In some cases, the method further comprises extending the primer to generate a complementary strand of the target polynucleotide. In some cases, extending is performed in the vessel or the hardened particle. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. In some cases, the reactive group is an N-hydroxysuccinimide (NHS) ester, a maleimide group, a hydrazide group, an azide group, or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine, adenine, cytosine or thymine. In some cases, the immobilizable probe comprises an immobilization moiety. In some cases, the immobilization moiety links the immobilizable probe to one or more of the polymers and/or monomers of the plurality. In some cases, the immobilization moiety a chemical that is co-polymerized with the polymers and/or monomers, a chemical or protein that stably interact with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the method further comprises contacting the plurality of vessels or hardened particles to a lysing agent. In some cases, the lysing agent comprises IGEPAL CA-630, Sarkosyl, protease K, lyticase, glusulase and zymolyase, or any combination thereof. In some cases, the barcode sequence diffuses into the plurality of hardened particles. In some cases, the vessel or the hardened particle further comprises a barcode-accepting polynucleotide. In some cases, the barcode is attached to the barcode-accepting polynucleotide. In some cases, the barcode-accepting polynucleotide is the target, the complementary strand of the target, or the immobilizable probe. In some cases, the vessel or the hardened particle further comprises a detection probe. In some cases, the barcode-accepting probe is attached to the detection probe. In some cases, generating the barcode sequence comprises: (a) pooling the plurality of hardened particle; (b) dividing the plurality of hardened particle into separate first populations; (c) attaching a first oligonucleotide comprising a first barcode sequence segment to the separate first populations, wherein each separate first population comprises a different first barcode sequence segment; (d) pooling the separate first populations to provide a first pooled population; (e) separating the first pooled population into a plurality of second populations; (0 attaching a second oligonucleotide comprising a second barcode sequence segment to the first oligonucleotide already attached to the second populations, wherein each of the plurality of second populations comprises a different second barcode sequence segment; and (g) pooling the plurality of second populations to provide a second pooled population comprising a barcode library. In some cases, the first barcode sequence segment or the second barcode sequence segment comprises at least 1 nucleotide in length. In some cases, the first barcode sequence segment or the second barcode sequence segment comprises at least 4 nucleotides in length. In some cases, the first barcode sequence segment or the second barcode sequence segment comprises from about 4 to about 20 nucleotides in length. In some cases, the barcode sequence is attached to the immobilizable probe, the target, or the complementary strand of the target polynucleotide. In some cases, the method further comprises washing the hardened particles extensively. In some cases, the single complex of cells comprises a first cell and a second cell. In some cases, the first cell is a mammalian cell. In some cases, the mammalian cell expresses a T-cell receptor or a portion thereof. In some cases, the first cell is an immune cell. In some cases, the immune cell is a T cell. In some cases, the second cell is an antigen presenting cell. In some cases, the second cell is a yeast cell. In some cases, the yeast cell expresses a MHC molecule on its surface. In some cases, the MHC molecule is a class I MHC or a class II MHC. In some cases, the MHC molecule is expressed from a gene selected from HLA-A, HLA-B, HLA-C, HLA-DPA1, HLA-DPB1, HLA-DQA1, HLA-DQB1, HLA-DRA, HLA-DRB1, or any combination thereof. In some cases, the MHC molecule further comprises a peptide. In some cases, the method further comprises sequencing the barcoded target sequence. In some cases, the vessel is a water-in-oil droplet. In some cases, the hardened particle is a hydrogel particle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 illustrates an example scheme of vessel formation and hardened particle generation. In this example, the hardened particle is immobilized with target nucleic acid molecules within its framework.

FIG. 2 illustrates an example of microfluidic channels.

FIG. 3 illustrates an example scheme of combinatorial split-and-pool barcode synthesis.

FIG. 4A illustrates an example of barcode-containing segment constructs that can bind to a barcode-accepting polynucleotide. A first barcode-containing segment carries a barcode sequence BC-a*. A second barcode-containing segment carries a barcode sequence BC-b*.

FIG. 4B illustrates an example of barding scheme using the barcode-containing segments illustrated in FIG. 4A.

FIG. 4C illustrates an example of a polynucleotide as an immobilizable probe.

FIG. 4D illustrates an example of an immobilizable probe. In this example, the immobilizable probe comprises two polynucleotide strands.

FIG. 5A illustrates a schematic representation of an immobilizable probe comprising immobilization moiety X and targeting moiety Y.

FIG. 5B illustrates an example of an immobilizable probe functioning in a vessel to link a target (T) onto the polymer framework. A barcode is attached to a barcode-accepting polynucleotide.

FIG. 6A illustrates an embodiment of the present disclosure wherein a target is a protein. A barcode-accepting polynucleotide is attached to a detection probe (e.g. antibody).

FIG. 6B illustrates an embodiment of the present disclosure wherein a target is a modified DNA.

FIG. 6C illustrates an embodiment of the present disclosure wherein a target is a chromosomal DNA associated with histones.

FIG. 6D illustrates an embodiment of the present disclosure wherein a target is an active protein (e.g. an active enzyme).

FIG. 7 illustrates images of hydrogel particles with or without stably entrapped oligonucleotide.

FIG. 8 illustrates an example of melting hydrogel particles using a reducing agent.

FIG. 9A illustrates an example scheme of ligating a barcode onto the barcode acceptor site of a barcode accepting polynucleotide. “dT20” is disclosed as SEQ ID NO: 16.

FIG. 9B illustrates an example scheme of generating another barcode acceptor site. “dT20” is disclosed as SEQ ID NO: 16.

FIG. 9C illustrates an example scheme of ligating another barcode onto the barcode acceptor site generated in FIG. 9B. “dT20” is disclosed as SEQ ID NO: 16.

FIG. 9D illustrates an example of generating another barcode acceptor site. “dT20” is disclosed as SEQ ID NO: 16.

FIG. 9E illustrates an example of ligating another barcode onto the barcode acceptor site generated in FIG. 9D. “dT20” is disclosed as SEQ ID NO: 16.

FIG. 10 illustrates experimental data of generating barcodes using hardened particles.

FIG. 11 illustrates experimental data of entrapping target molecules in hardened particles.

### DETAILED DESCRIPTION OF THE INVENTION

In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

Overview

Single cells can be encapsulated in water-in-oil droplets of heterogeneous or uniform size. Many droplets may contain one and only one cell. If the aqueous solution in the droplets contains precursors of the hydrogel (e.g., polymerizable monomers or cross-linkable linear polymers) and the precursors can be converted to hydrogel form after the formation of the droplets, then a hydrogel particle entrapping a cell can be formed in each droplet that contains a cell. Such particles can later be purified away from the oil phase and be dispersed in aqueous solution. These particles can undergo additional manipulations such as flow cytometry analysis, FACS sorting, microscopic analysis, or even re-encapsulation into water-in-oil droplets. The hydrogel particle may provide structural support for the embedded cell which may facilitate analysis of the cell with single-cell resolution.

The target molecules (such as nucleic acids and proteins) within the entrapped cells can be manipulated. One can deliver reagents (such as enzymes, probes and primers) to the target molecules. In these cases, the cell membrane may be permeabilized. However, during this permeabilization process, some target molecules may leak out of the cell and out of the hydrogel particle. Target molecules of smaller molecular weight and diameter may be more likely to leak out; whereas large structures (such as cytoskeleton and chromatin) may be retained. This leakage can pose a challenge to single-cell analysis. Not only one may lose molecules of interest in one hydrogel particle, the molecule of interest may also diffuse to other hydrogel particles, causing confusing results.

The root cause of the leakage problem may be that, although the cell is entrapped in the hydrogel particle, the target molecule may not have stable association with the hydrogel particle. Thus, to solve this leakage problem, one may engineer stable molecular interaction between the target molecule and the polymer network that support the hydrogel. The original (or primary) target molecule may be converted to a secondary target molecule. For example, an mRNA molecule may be converted to a cDNA molecule via reverse transcription, where the mRNA molecule can be the primary target molecule and the cDNA molecule can be the secondary target molecule. As another example, a protein may be converted to a nucleic acid molecule via proximity ligation assay (PLA), where the protein can be the primary target molecule and the nucleic acid molecule can be the secondary target molecule.

In analyzing mRNAs in a population of cells, a polyT-based reverse transcription (RT) primer with an Acrydite modification and 3 to 10% polyacrylamide hydrogel may be used. The polyT-based RT primer may comprise 20 to 30 consecutive dT nucleotides optionally followed by a dV (dA, dC, or dT) nucleotide at its 3′ end (SEQ ID NO: 1). The RT primer can be co-polymerized into the polyacrylamide framework that support the hydrogel, and can either stably bind the target mRNA molecules, or be part of the cDNA (the secondary target molecule). The target molecule can be stably attached to the hydrogel, yet the hydrogel can be sufficiently porous to allow reagents to diffuse to the target molecule.

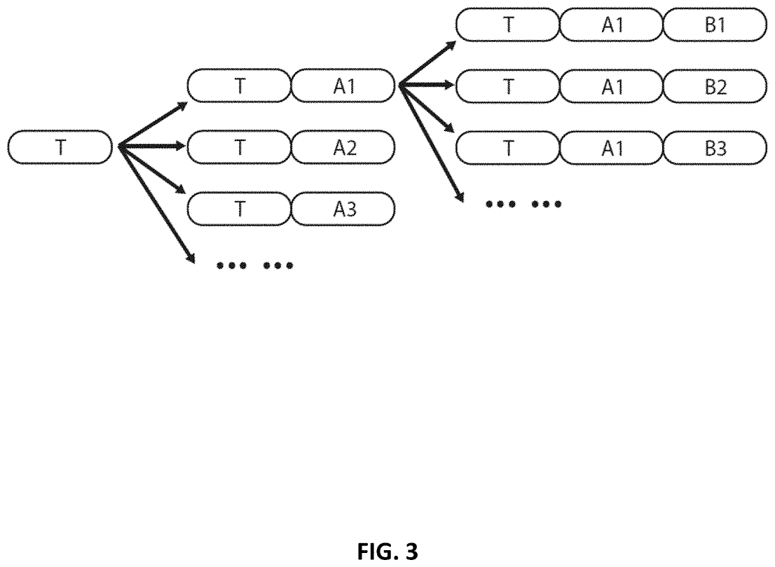

Provided herein are compositions and methods to entrap a target in a hardened particle (e.g. a hydrogel particle) and barcode the target for downstream applications. The target provided herein can be a polynucleotide, a polypeptide, or an organelle from a single cell, a single complex of cell or a single exosome. The present disclosure provides various strategies to immobilize or restrict diffusion of the target within a hardened particle so that the target can be stably entrapped or embedded. A split-and-pool combinatorial strategy described herein can be used to barcode each hardened particle so that one or more targets from the same hardened particle can be barcoded with a common barcode sequence (e.g., FIG. 3). The barcoded sequences can further be analyzed in a downstream application, for example, sequencing. The compositions and methods provided herein allow for highly efficient barcoding and increased percentage of barcoded hardened particles with targets entrapped therein. The methods provided herein can ensure every single target of interest will be barcoded and therefore can be analyzed, resulting in high barcoding efficiency. The hardened particle comprising a target entrapped or embedded therein also allows for vigorous downstream manipulations or sample preparations (e.g. extensive washing).

An example of the barcoding methods described herein can comprise: (a) forming a plurality of vessels each comprising: (i) a plurality of polymerizable or gellable polymers and/or monomers; and (ii) a target or derivative thereof from a single cell, a single complex of cells, or a single exosome; (b) polymerizing or gelling the polymers and/or monomers in each of the plurality of vessels to form a hardened (e.g. polymerized or gelled) particle, thereby forming a plurality of hardened particles; (c) generating a barcode sequence in each of the plurality of hardened particles, thereby generating a barcoded target sequence.

An example of the compositions described herein can be a hardened particle, comprising: a polymerized or gelled plurality of polymers and/or monomers; a single cell, a single complex of cells, or a single exosome; an immobilizable probe linked to one or more polymers and/or monomers of the polymerized or gelled plurality; and a barcode-accepting polynucleotide. The barcode-accepting polynucleotide can be used to attach a barcode sequence.

### Definitions

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polynucleotide”, “nucleotide”, “nucleic acid” and “oligonucleotide” are used interchangeably. They can refer to a polymeric form of nucleotides of any length, either deoxyribonucleotides or ribonucleotides, or analogs thereof. A polynucleotide may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), circular RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. In some cases, a polynucleotide provided herein is a target, e.g. a target polynucleotide.

Polynucleotides may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K et al., Nat. Chem. Biol. 2012 July; 8(7):612-4, which is herein incorporated by reference for all purposes.

As used herein, the term “polypeptide” is a polymer of amino acids and which are joined together through amide bonds and is alternatively referred to as a “peptide”. In the context of this specification it should be appreciated that the amino acids may be the L-optical isomer or the D-optical isomer. Peptides are two or more amino acid monomers long, and often can be more than 20 amino acid monomers long. A polypeptide can be linearly unstructured or folded in three-dimensional structure. A structured polypeptide can be a protein. As used herein, “protein” refers to a long polymer of amino acid residues linked via peptide bonds and which may be composed of one or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, antibodies, and any fragments thereof. A protein can be a portion of the protein, for example, a domain, a subdomain, or a motif of the protein. A protein can be a variant (or mutation) of the protein, wherein one or more amino acid residues are inserted into, deleted from, and/or substituted into the naturally occurring (or at least a known) amino acid sequence of the protein. A protein can be a modified protein. Non-limiting examples of protein modification include phosphorylation, acetylation, glycosylation, amidation, hydroxylation, methylation, alkylation, acylation, ubiquitylation, pyrrolidone carboxylic acid, and sulfation. A protein or a variant thereof can be naturally occurring or recombinant.

The term “sequence” and its grammatical equivalents as used herein can refer to a polypeptide sequence or a polynucleotide sequence. A polynucleotide sequence can be DNA or RNA; can be linear, circular or branched; and can be either single-stranded or double stranded. A sequence can be mutated. A sequence can be of any length, for example, between 2 and 1,000,000 or more amino acids or nucleotides in length (or any integer value there between or there above), e.g., between about 100 and about 10,000 nucleotides or between about 200 and about 500 amino acids or nucleotides.

The term “vessel” used herein refers to a compartment (e.g. a microfluidic channel, a well, or a droplet) in which a biochemical reaction (e.g., target protein and antibody binding, nucleic acid hybridization and primer extension) may occur. The terms “vessel” and “compartment” can be used interchangeably. The volume of the compartment may be as large as 1 mL or as small as 1 picoLiter. In some embodiments, the median size of the compartments of a plurality of compartments is from 1 to 10 picoLiter, from 10 to 100 picoLiter, from 100 picoLiter to 1 nanoLiter, from 1 to 10 nanoLiter, from 10 to 100 nanoLiter, from 100 nanoLiter to 1 microLiter, from 1 to 10 microLiter, from 10 to 100 microLiter, or from 100 to 1000 microLiter. The volume of the aqueous content in the compartment can be smaller than or about equal to the volume of the compartment. In some embodiments, the median volume of the aqueous content in the compartments is 1 microLiter or less. The vessel or compat intent can comprise a plurality of polymerizable or gellable polymers and/or monomers. The plurality of polymerizable or gellable polymers and/or monomers forms a hydrogel or hardened matrix upon polymerization or gelation, thereby forming a hardened particle. In some cases, a vessel comprises a hardened particle. In some cases, the hardened particle is a hardened vessel. The hardened particle can be a polymerized or gelled particle. The hardened particle can be a bead. The hardened particle can be a porous particle. The hardened particle can be a hydrogel particle. The hydrogel particle may be made of gelled polymers such as cross-linked polyacrylamide, cross-linked PEG, agarose, or alginate.

“Droplets” are compartments surrounded by liquid rather than solid. Droplets may be water-in-oil; water-in-oil-in-water, or water in a lipid layer (liposome). In some embodiments, droplets can be of uniform size or heterogeneous size. In some embodiments, the median diameter of the droplets in a plurality of droplets can range from about 0.001 μm to about 1 mm. In some embodiments, the median volume of the droplets in a plurality of droplets can range from 0.01 nanoLiter to 1 microLiter.

The term “particle” includes a large number of insoluble materials of any configuration, including spherical, thread-like, brush-like and many irregular shapes. Particles can be porous with regular or random channels inside. Examples include silica, cellulose, Sepharose beads, polystyrene (solid, porous and derivatized) beads, controlled-pore glass, gel beads, sols, biological cells, subcellular particles, microorganisms (protozoans, bacteria, yeast, viruses, etc.) micelles, liposomes, cyclodextrins, two phase systems (e.g. agarose beads in wax) and other structures which entrap or encapsulate a material.

The term “partition,” as used herein, may be a verb or a noun. When used as a verb (e.g., “to partition,” or “partitioning”), the term generally refers to the fractionation (e.g., subdivision) of a species or sample (e.g., a polynucleotide) between vessels that can be used to sequester one fraction (or subdivision) from another. Such vessels are referred to using the noun “partition.” Partitioning may be performed, for example, using microfluidics, dilution, dispensing, and the like. A partition may be, for example, a well, a microwell, a hole, a droplet (e.g., a droplet in an emulsion), a continuous phase of an emulsion, a test tube, a spot, a capsule, a bead, a surface of a bead in dilute solution, or any other suitable container for sequestering one fraction of a sample from another. A partition may also comprise another partition.

“Polymerizable or gellable polymer or monomer” refers to any polymer or monomer that is capable of forming a hydrogel or a hardened matrix through a polymerization or a non-polymerization mechanism. Polymerizable or gellable polymers suitable for use in the present disclosure are those which are soluble or dispersible in an aqueous liquid to increase the viscosity of the liquid. Polymerizable or gellable polymers include those which are capable of crosslinking with a suitable crosslinking agent via crosslinkable groups. The “polymerizable” may encompass the meaning of “crosslinkable”. Polymerization can be a process of polymer formation from monomers, and can also be a process of crosslinked polymer formation from linear polymers. The polymerizable polymer can be a macromer. The term “macromer”, as used herein, refers to any polymer or oligomer that has a functional group that can take part in further polymerization.

“Matrix”, “framework”, and “polymer framework” can be used interchangeably herein and refer to the polymer network formed within a vessel or compartment.

Biological Particle

The analysis of a single biological particle can comprise analyzing biological contents originated from the single biological particle. For example, a single-cell analysis is to study of genomics, transcriptomics, proteomics and metabolomics at the single cell level. Partitioning a single cell or single complex of cells into an individual compai intent is useful for single-cell analysis. In various embodiments, the methods provided herein comprise generating a plurality of vessels or compartments, wherein each of the plurality of vessels or compartments comprises a single biological particle.

Biological particles are individually separable and dispersible particles of biological origin, such as cells (prokaryotic or eukaryotic), nuclei, organelles (such as mitochondria), and viruses. In some embodiments, biological particles are prepared from biological samples. For example, the biological particles can be cells prepared from fresh tissue (such as dense cell matter from tumor or neural tissues). In some embodiments, biological particles are whole cells or nuclei prepared from frozen tissue. In some embodiments, biological particles are nuclei (rather than cells). In some embodiments, the biological particles are nuclei prepared from FFPE tissue.

In some embodiments, a single biological particle is a single cell, a single complex of cells, a single exosome, a single organelle, or any combination thereof. The single cell can be any type of cell, including normal and cancer cells. In some case, the single cell is an immune cell, for example, a B cell or a T cell. After partitioning a single biological particle into an individual compartment, the biological particle can be manipulated to release its constituents. For example, a single cell or a single complex of cells can be lysed to release its DNAs, RNAs, proteins, and/or peptides into the compartment for further analysis.

In some embodiments, a single biological particle is a single polypeptide molecule. In some embodiments, a single biological particle is a single polynucleotide molecule. In some embodiments, a single biological particle is a single molecule. In some embodiments, a single biological particle is one or more molecules, wherein each of the one or more molecules is from a single cell, a single complex of cells or a single exosome.

In some embodiments, the methods provided herein can be used to generate a plurality of vessels or compartments, wherein each vessel or compartment of the plurality of vessels or compartments comprises a single biological particle. In some embodiments, the methods provided herein can be used to generate a plurality of vessels or compartments, wherein each vessel or compartment of the plurality of vessels or compartments comprises a single complex of cells. An example of analyzing a single complex of cells includes, but is not limited to, analyzing paired T cell and antigen presenting cell conjugates. The polynucleotide sequences from the T cell and antigen presenting cell conjugate within the compartment or vessel can be labeled with a unique barcode sequence for further sequencing analysis. The polynucleotide sequences can comprise the polynucleotide sequences encoding the T cell receptor. The antigen presenting cell may further comprise a polynucleotide sequence encoding a peptide that can be represented by a MHC molecule on the cell surface of the antigen presenting cell. The polynucleotide sequences analyzed can comprise the polynucleotide sequence encoding the peptide. The single complex of cells can comprise a first cell and a second cell. The first cell can be a mammalian cell. In some embodiments, the mammalian cell expresses a T cell receptor or a portion thereof. In some embodiments, the first cell is an immune cell. In some embodiments, the immune cell is a T cell. In some embodiments, the second cell is an antigen presenting cell or dendritic cell. In some embodiments, the second cell is a mammalian cell. In some embodiments, the second cell is a B cell. In some embodiments, the second cell is a yeast cell. In some embodiments, the yeast cell expresses a MHC molecule on its surface. In some embodiments, the MHC molecule is a class I MHC or a class II MHC. In some embodiments, the MHC molecule is expressed from a gene selected from HLA-A, HLA-B, HLA-C, HLA-DPA1, HLA-DPB1, HLA-DQA1, HLA-DQB1, HLA-DRA, HLA-DRB1, or any combination thereof. In some embodiments, the MHC molecule further comprises a peptide. The peptide can be an antigen or neoantigen that can be recognized by a T cell.

An aspect of the disclosure provides the methods to provide biological particles (i.e., prepare biological particles in such a way as to ready them for compartmentalization). In some embodiments, the number of provided biological particles can be about 1, 2, 3, 4, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of provided biological particles can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of provided biological particles can be less than about 5, 10, 50, 100, 250, 500, 750, 1000, 1500, 2000, 2500, 5000, 7500, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of provided biological particles can be about 5-10000000, 5-5000000, 5-1000000, 10-10000, 10-5000, 10-1000, 1000-6000, 1000-5000, 1000-4000, 1000-3000, or 1000-2000.

In some embodiments, the biological particles do not need to be prepared beyond standard washing and incubation, for example, if they are cells in suspension such as peripheral blood mononuclear cells (PBMCs) and/or pre-dissociated cells. In some embodiments, the biological particles need to be prepared into suspension.

In some embodiments, the biological sample is dissociated by mechanical means. Mechanical separation can be serial passage through a constrictive device such that shearing forces pull biological particles apart. A constrictive device can be a large bore pipet tip, a Pasteur pipet, a Dounce homogenizer, or similar. Mechanical separation can be achieved by passing the biological particles through the constriction a number of times. The number of passages can be about 1, 2, 3, 4, 5, 6, 8, 10, 15, 20, 25, 30, 40, 50, or more. The number of passages can be at least about 1, 2, 3, 4, 5, 6, 8, 10, 15, 20, 25, 30, 40, 50, or more. The number of passages can be less than about 1, 2, 3, 4, 5, 6, 8, 10, 15, 20, 25, 30, 40, 50, or more. The number of passages can be about 1 to 50, 1 to 10, 2 to 10, 5 to 10, 5 to 20, 5 to 30, 10 to 20, 10 to 30, 10 to 40, 15 to 20, 15 to 30, 20 to 30, 20 to 40, 20 to 50, or 30 to 50.

In some embodiments, fixation reversal agent is used to facilitate the dissociation of the biological sample. A fixation reversal agent can be used to reverse the connective material in fixed cells. A fixation reversal agent may include, but not limited to, a fixation reversal enzyme or a fixation reversal catalyst. A fixation reversal enzyme is an enzyme that digests some content of the fixed biological sample so that the target nucleic acid is more accessible for analysis. For example, mRNA in formalin-fixed biological samples may be inaccessible for reverse transcription primers or enzymes due to the heavy crosslinking of the protein contents in the biological sample. Fixation reversal enzymes, such as proteinase K, collagenase, and hyaluronidase, can digest some protein and/or carbohydrate content of the fixed biological sample, making mRNA more accessible. A fixation reversal catalyst is catalyst that aids in the reversal of the fixation. For example, the fixation reversal catalyst can include bifunctional transimination catalysts such as anthranilates and/or phosphoanilates that catalyze the reversal of adducts formed during formalin fixation. Examples of the fixation reversal agent can include, but not limited to, collagenase, hyaluronidase, and trypsin. The fixation reversal agent can be a combination of agents. The fixation reversal agent can be provided in the amount suggested by the manufacturer to digest a given amount of substrate for a given time and temperature. The biological particle can be treated with about 1, 2, 3, 4, 5, 10, 15, 20, 25, 30, 50, 100, 150, 200, 250, or 500 times the amount suggested for the estimated content in the sample. The biological particle can be treated with at least about 1, 2, 3, 4, 5, 10, 15, 20, 25, 30, 50, 100, 150, 200, 250, or 500 times the amount suggested for the estimated content in the sample. The biological particle can be treated with less than about 1, 2, 3, 4, 5, 10, 15, 20, 25, 30, 50, 100, 150, 200, 250, or 500 times the units suggested for the estimated content in the sample. The temperature for incubation can be about 20, 25, 30, 35, 37, 40, 45, 50, 55, 60, 65, 70, or 75° C. The temperature for incubation can be at least about 20, 25, 30, 35, 37, 40, 45, 50, 55, 60, 65, 70, or 75° C. The temperature for incubation can be less than about 20, 25, 30, 35, 37, 40, 45, 50, 55, 60, 65, 70, or 75° C. The time can be about 5, 10, 15, 20, 25, 30, 40, 45, 50, 55 minutes or 1, 1.5, 2, 2.5, 3, 4, 5, 6, 7, 8, 10, 12, 14, 16, 18, 20, or 24 hours. The time can be at least about 5, 10, 15, 20, 25, 30, 40, 45, 50, 55 minutes or 1, 1.5, 2, 2.5, 3, 4, 5, 6, 7, 8, 10, 12, 14, 16, 18, 20, or 24 hours. The time can be less than about 5, 10, 15, 20, 25, 30, 40, 45, 50, 55 minutes or 1, 1.5, 2, 2.5, 3, 4, 5, 6, 7, 8, 10, 12, 14, 16, 18, 20, or 24 hours.

In some embodiments, chemicals are used to facilitate the dissociation of the biological sample. The chemical used can be a denaturant, such as urea or guanidinium, or a chelating agent, such as EDTA. The chemical can also be a detergent, such as Triton X-100, Tween 20, Nonident P40 (NP40), IGEPAL CA-630, or similar. The concentration of a chemical can be about 1, 2, 5, 10, 15, 20, 30, 50, 100, 200, 300, 500, 1000, or 2000 mM in water or buffer. The concentration of a chemical can be at least about 1, 2, 5, 10, 15, 20, 30, 50, 100, 200, 300, 500, 1000, or 2000 mM in water or buffer. The concentration of a chemical can be less than about 1, 2, 5, 10, 15, 20, 30, 50, 100, 200, 300, 500, 1000, or 2000 mM in water or buffer. The concentration of detergent can be about 0.1, 0.5, 1, 2, 3, 4, 5, 6, 8, 10, 15, 20, 25, or 30 percent volume/volume (% v/v) in water or buffer. The concentration of detergent can be at least about 0.1, 0.5, 1, 2, 3, 4, 5, 6, 8, 10, 15, 20, 25, or 30% v/v in water or buffer. The concentration of detergent can be less than about 0.1, 0.5, 1, 2, 3, 4, 5, 6, 8, 10, 15, 20, 25, or 30% v/v in water or buffer. The concentration of detergent can be about 0.1 to 30, 0.1 to 1, 0.1 to 5, 1 to 5, 0.5 to 1, 0.5 to 2, 0.5 to 5, 1 to 10, 5 to 10, 2 to 8, 5 to 20, 5 to 30, 10 to 20, or 10 to 30% v/v in water or buffer. The temperature of heating can be about −80, −70, −50, −20, −10, −5, −1, 20, 25, 30, 35, 37, 40, 45, 50, 55, 60, 65, 70, or 75° C. The temperature of heating can be at least about −80, −70, −50, −20, −10, −5, −1, 20, 25, 30, 35, 37, 40, 45, 50, 55, 60, 65, 70, or 75° C. or greater. The temperature of heating can be less than about −80, −70, −50, −20, −10, −5, −1, 20, 25, 30, 35, 37, 40, 45, 50, 55, 60, 65, 70, or 75° C. The temperature of heating can be about −80 to 100° C., −80 to 20° C., −20 to 0° C., 0 to 20° C., 0 to 37° C., 20 to 100° C., 20 to 75° C., 50 to 75° C., 30 to 50° C., 40 to 75° C., 75 to 100° C., or 75 to 90° C. The time of heating can be about 5, 10, 15, 20, 25, 30, 40, 45, 50, or 60 minutes. The time can be about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time of heating can be at least about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time for heating can be less than about 5 minutes, 15 minutes, 30 minutes, 45 minutes, or 1, 1.5, 2, 2.5, 3, 4, 5, 6, 7, 8, 10, 12, 14, 16, 18, 20, or 24 hours. In some embodiments, the chemical is a combination of denaturant, chelator, and/or detergent.

In some embodiments, the sample is dissociated with targeted separation. In targeted separation, a microscope or visual aid is used to select individual cells from tissue in a manual or automated fashion. An example is laser capture microdissection.

In some embodiments, the sample dissociation may be incomplete. Incomplete dissociation can be a mixed suspension of single cells and intact tissue. The mixture can be partitioned by filtering. The filter can be about a 10, 20, 30, 35, 40, 50, 70, or 100 μm nylon mesh.

In some embodiments, the sample is dissociated by a combination of dissociation methods. In some embodiments, this can be enzymatic treatment of a biological sample followed by mechanical separation of individual particles. In some embodiments, as with very difficult preserved tissue, the sample may be washed in a solvent.

In some embodiments, the dissociated sample may be enriched for a specific population or multiple populations by FACS, MACS, or similar.

Target

The present disclosure provides compositions and methods for analyzing a target, including barcoding a target for downstream sequencing analysis. The target can be a target polynucleotide, a target polypeptide, a target organelle, or a combination thereof. The target can be a target polynucleotide, a target polypeptide, or a combination thereof, from a single biological particle. The target can be a target polynucleotide, a target polypeptide, or a combination thereof, from a single cell, a single complex of cells, or a single exosome. The target can be any type of biological contents released from a single biological particle, including a single cell, a single complex of cells, or a single exosome. In many cases, the target can be a target molecule. The target from a single biological particle can be entrapped into an individual compartment or vessel for barcoding and further analysis.

The single biological particle partitioned in an individual vessel can be treated (e.g. lysed) to release its biological contents comprising target molecules. The biological particle can be lysed before gelling or after gelling of the polymerizable or gellable polymers and/or monomers within the vessel. For example, the biological particle can be lysed within a droplet of an emulsion. The lysing buffer or agent can be co-compartmentalized with the biological particle into an individual droplet. For another example, the biological particle can be lysed within a hardened particle. The lysing buffer or agent can be brought into contact with the hardened particle, allowing the lysing buffer or agent diffuses into the hardened particle through pores.

To entrap the target within a hardened particle, the target can be immobilized within the matrix of the hardened particle to prevent the target from diffusing out of the hardened particle. The immobilization can be achieved through an immobilizable probe which binds to the target and also interacts with the matrix of the hardened particle. The immobilizable probe can bind to the matrix through covalent bond or non-covalent interaction. The immobilizable probe can comprise (e.g. be conjugated with) one or more high molecular weight polymer tails (e.g. polyethylene glycol with molecular weight of 3350, 8000, and 20,000) which result in a higher hydrodynamic radius than the pore size of the hardened particle. A total molecular weight of the conjugated polymer tails (or chains) can be from 5 kDa to 1000 kDa. In some cases, the total molecular weight of the conjugated polymer tails can be from 5 kDa to 10 kDa, from 10 kDa to 15 kDa, from 15 kDa to 20 kDa, from 20 kDa to 25 kDa, from 25 kDa to 30 kDa, from 30 kDa to 35 kDa, from 35 kDa to 40 kDa, from 40 kDa to 45 kDa, or from 45 kDa to 50 kDa. In some cases, the total molecular weight of the conjugated polymer tails can be from 50 kDa to 100 kDa, from 100 kDa to 150 kDa, from 150 kDa to 200 kDa, from 200 kDa to 250 kDa, from 250 kDa to 300 kDa, from 300 kDa to 350 kDa, from 350 kDa to 400 kDa, from 400 kDa to 450 kDa, or from 450 kDa to 500 kDa. In some cases, the total molecular weight of the conjugated polymer tails can be from 500 kDa to 600 kDa, from 600 kDa to 700 kDa, from 700 kDa to 800 kDa, from 800 kDa to 900 kDa, from 900 kDa to 1,000 kDa, from 1,000 kDa to 1,500 kDa, from 1,500 kDa to 2,000 kDa, from 2,000 kDa to 3,000 kDa, or from 3,000 kDa to 5,000 kDa. The immobilizable probe may also bind to a large particle which is in turn entrapped within the hardened particle. The large particle usually has a size that is larger than the pore size of the hardened particle. One can should a large particle, and adjust the pore size of the hardened particle accordingly. The size of the large particle may be from 0.5 μm to 5 μm. In some cases, the size of the large particle may be from 0.5 μm to 1 μm, from 1 μm to 1.5 μm, from 1.5 μm to 2 μm, from 2 μm to 2.5 μm, from 2.5 μm to 3 μm, from 3 μm to 3.5 μm, from 3.5 μm to 4 μm, from 4 μm to 4.5 μm, from 4.5 μm to 5 μm. In some other cases, the size of the large particle may be from 5 μm to 10 μm, from 10 μm to 20 μm, from 20 μm to 30 μm, from 30 μm to 40 μm, or from 40 μm to 50 μm.

In some cases, the target is a target polynucleotide. According to one aspect, target polynucleotides are single stranded nucleic acids. According to another aspect, target polynucleotides are double stranded nucleic acids. The target polynucleotide can be DNA, for example, genomic DNA. The target polynucleotide can be modified DNA. An example of modified DNA includes, but is not limited to, methylated DNA. The methylated DNA can comprise 5-methyl-cytosine, 5-hydroxymethylcytosine, 5-formylcytosine, 5-carboxylcytosine, CpG dinucleotides, or non-CpG methylation. The target polynucleotide can be RNA, including, e.g., mRNA, total RNA, circular RNA, non-coding RNA, microRNA, ribosomal RNA, transfer RNA, small interfering RNA (siRNA), small nucleolar RNA (snoRNAs), Piwi-interacting RNA (piRNA), tRNA-derived small RNA (tsRNA), small rDNA-derived RNA (srRNA), Transfer-messenger RNA (tmRNA), or the like, that may be processed to produce cDNA for sequencing, e.g., using any of a variety of RNA-seq methods. In some cases, the target polynucleotide encodes a T cell receptor (TCR) alpha subunit, a TCR beta subunit, a TCR gamma subunit, or a TCR delta subunit. The length of the target polynucleotide can vary. According to certain aspects, the length of the target polynucleotide can be from 1 nucleotide to about 3,000,000 nucleotides in length, from 1 nucleotide to about 2,500,000 nucleotides in length, from 1 nucleotide to about 2,000,000 nucleotides in length, from 1 nucleotide to about 1,500,000 nucleotides in length, from 1 nucleotide to about 1,000,000 nucleotides in length, from 1 nucleotide to about 500,000 nucleotides in length, from 1 nucleotide to about 250,000 nucleotides in length, from 1 nucleotide to about 200,000 nucleotides in length or from 1 nucleotide to about 150,000 nucleotides in length. Examples of target polynucleotide can also be from 1 nucleotide to about 100,000 nucleotides in length, from 1 nucleotide to about 10,000 nucleotides in length, from 1 nucleotide to about 5,000 nucleotides in length, from 4 nucleotides to about 2,000 nucleotides in length, from 6 nucleotides to about 2,000 nucleotides in length, from 10 nucleotides to about 1,000 nucleotides in length, from 10 nucleotides to about 500 nucleotides in length, from 10 nucleotides to about 300 nucleotides in length, from 10 nucleotides to about 200 nucleotides in length, from 20 nucleotides to about 100 nucleotides in length, and any range or value in between whether overlapping or not.

In some cases, the target is a target polypeptide. The target polypeptide can be unstructured or structured, including peptides and proteins. The target polypeptide can be a domain or a motif of a protein. The target polypeptide can comprises one or more polypeptide chains. The target polypeptide can be a protein complex comprising two or more proteins. The target polypeptide can be a protein with any function, including enzymes, structural proteins, and ligand binding and signal transduction proteins. The target polypeptide can be a secreted protein, a transmembrane protein, or an intracellular protein. The intracellular protein can localize in any subcellular location within a cell, including but not limited to ER, Golgi, lysosomes or vacuoles, mitochondria, chloroplasts, and plasma membrane. The target can be total proteins within a given cell.

A single chain of the target polypeptide can be in any length. For example, the single chain of a target polypeptide can be from 2 to 50000 amino acid residues in length. In some embodiments, a single chain of a target polypeptide can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, a single chain of a target polypeptide can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500 or more amino acid residues in length. In some embodiments, a single chain of a target polypeptide can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length.

In some embodiments, a single chain of a target polypeptide has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids. In some embodiments, a single chain of a target polypeptide has a total length of at least 500, at least 600, at least 700, at least 800, at least 900, at least 1,000, at least 1,200, at least 1,500, at least 2,000, at least 3,000, at least 4,000, at least 5,000, at least 6,000, at least 7,000, at least 8,000, at least 9,000, or at least 10,000 amino acid residues in length. In some embodiments, a single chain of a target polypeptide has a total length of at least 10,000, at least 20,000, at least 30,000, at least 40,000, or at least 50,000 amino acid residues in length.

In some embodiments, a single chain of a target polypeptide has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids. In some embodiments, a single chain of a target polypeptide has a total length of at most 500, at most 600, at most 700, at most 800, at most 900, at most 1,000, at most 1,200, at most 1,500, at most 2,000, at most 3,000, at most 4,000, at most 5,000, at most 6,000, at most 7,000, at most 8,000, at most 9,000, or at most 10,000 amino acid residues in length. In some embodiments, a single chain of a target polypeptide has a total length of at most 10,000, at most 20,000, at most 30,000, at most 40,000, or at most 50,000 amino acid residues in length.

In some cases, the target comprises both a polypeptide and a polynucleotide. For example, the target can comprise total mRNA and total proteins released from a single cell. In some cases, the target can be a complex formed by a polypeptide and a polynucleotide. For example, the target can be ribosome or a portion thereof which comprises both proteins and ribosomal RNA. For another example, the target can be chromosome or a portion thereof comprising histones and chromosomal DNA.

In some cases, the target can be a target organelle. The target organelle can be membrane-bound and non-membrane bound. The target organelle that are membrane-bound include, but is not limited to, nucleolus, nucleus, ribosome, vesicle, rough endoplasmic reticulum, golgi apparatus, cytoskeleton, smooth endoplasmic reticulum, mitochondrion, vacuole, cytosol, lysosome, centrosome, cell membrane, and plastids. The non-membrane bound organelles, also called large biomolecular complexes, are large assemblies of macromolecules that carry out particular and specialized functions, but they lack membrane boundaries. Such cell structures include: large RNA and protein complexes (e.g. ribosome, spliceosome, vault); large protein complexes (e.g. proteasome, DNA polymerase III holoenzyme, RNA polymerase II holoenzyme, symmetric viral capsids, complex of GroEL and GroES); membrane protein complexes (e.g. photosystem I, ATP synthase large DNA); and protein complexes (e.g. nucleosome centriole and microtubule-organizing center (MTOC), cytoskeleton, flagellum, nucleolus, stress granule, germ cell granule, neuronal transport granule). In some embodiments, the target organelle can comprise acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some embodiments, the target organelle can comprise carboxysome, chlorosome, flagellum, magnetosome, mesosomes, nucleoid, pilus, plasmid, ribosome (70S), thylakoid, or any combination thereof.

In some cases that organelles are isolated from cells before partitioning into individual compartments, the organelles can be considered as biological particles. In such cases, the organelles are partitioned into individual compartments or vessels, and the target can be biological contents released from a single organelle.

The target can be of a genus or of a species. For example, polynucleotide and polypeptide are of different genus. Within a genus, there can be different species. For example, target polynucleotide can comprise DNA species and RNA species. For another example, target polypeptide can comprise phosphorylated protein species and methylated protein species.

Immobilizable Probe

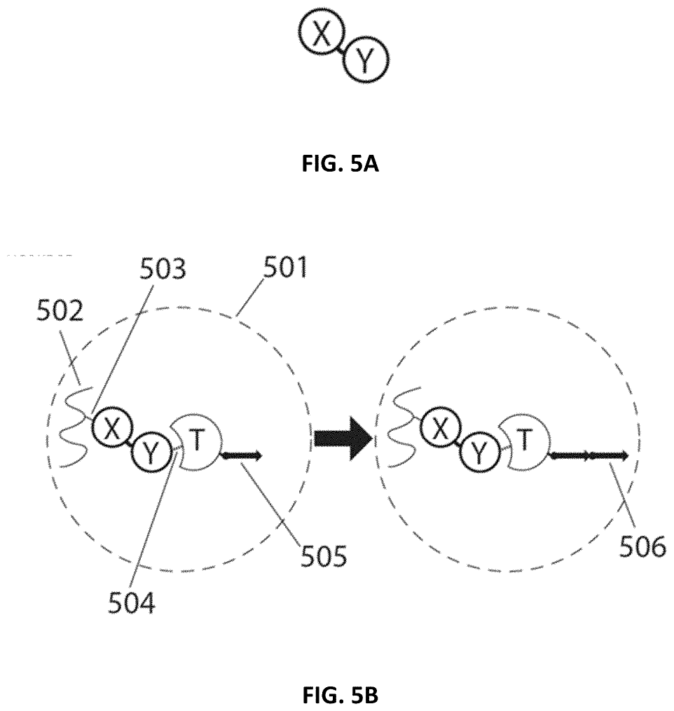

The compositions or methods provided herein comprise an immobilizable probe (e.g., FIGS. 5A and 5B). The immobilizable probe can function as an anchor to immobilize or entrap a target within a vessel or a hardened particle. On one hand, the immobilizable probe can bind to a target. On the other hand, the immobilizable probe can associate with the matrix or polymer framework within a hardened particle to restrict its own diffusion which in turn results in limited diffusion of the target.

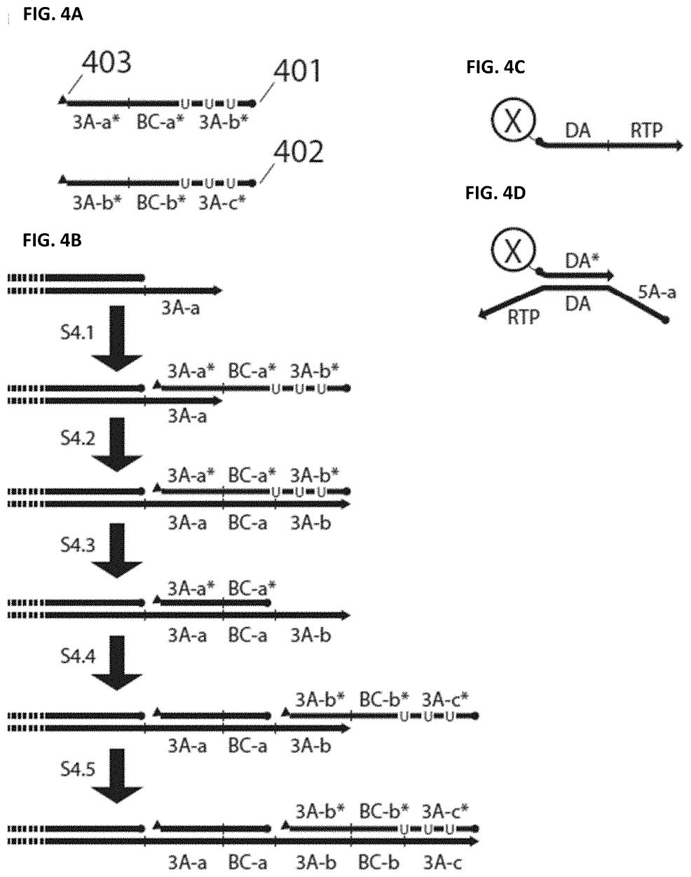

An immobilizable probe can comprise two moieties: a targeting moiety and an immobilization moiety. The immobilization moiety can be responsible for attaching the immobilizable probe to the polymer framework (e.g. matrix) supporting the hardened particle (e.g. hydrogel), and can be chosen based on the choice of the polymer framework. The targeting moiety can be responsible for making a stable interaction with the target. The immobilizable probe can be one molecule wherein the targeting moiety and the immobilization moiety are two portions of the same molecule and are covalently bound. The immobilizable probe can be more than one molecule, wherein the targeting moiety and the immobilization moiety can be covalently linked through a linker or non-covalently linked. For example, the immobilization moiety can be a first polynucleotide linked to the polymer framework, and the targeting moiety can be a second polynucleotide which can hybridize with the first polynucleotide (e.g. FIG. 4D). It is to be understood that there is no limitation on the type of interaction between the targeting moiety and the immobilization moiety of the immobilizable probe.

The targeting moiety may be specific of the ‘genus’ of the target molecule or the ‘species’ of the target molecule. For example, the genus of target molecules can be protein genus or polynucleotide genus. The targeting moiety can be a polynucleotide, a polypeptide, or a chemical group. To recognize a ‘genus’ of target molecule, the targeting moiety may bind to or bond with a general class of molecules such as proteins and nucleic acids. In some situations, the targeting moiety can be a chemical group. For example, the targeting moiety can be an NHS ester or a maleimide group, making the targeting moiety capable of forming covalent bonds with lysine or cysteine side chains, respectively, of essentially all proteins. As another example, the targeting moiety may comprise a “Label-IT” linker and reactive groups which can form covalent bond with guanine bases of essentially all DNA and RNA molecules. An example of immobilizable probe comprising an NHS ester as a targeting moiety can be “Acryloyl-X, SE, 6-((acryloyl)amino)hexanoic Acid, Succinimidyl Ester” (available from ThermoFisher, cat. #A20770), wherein the immobilizable probe can be co-polymerized with polyacrylamide gels via immobilization moiety.

To recognize a specific ‘species’ of target molecules, the targeting moiety often comprise an oligonucleotide, an antibody, an antibody fragment, or an aptamer that recognize specific sequence of 3D structure of the target molecule. Such sequence or 3D structure may be specific to one gene or protein, or maybe shared by multiple genes or proteins (e.g., poly A tail is shared by essentially all mRNA molecules). For example, when proteins are target molecules of interest, the targeting moiety can be an antibody specific for a protein target. For another example, when polynucleotides are target molecules of interest, the targeting moiety can be an oligonucleotide which hybridizes with a target polynucleotide. The targeting moiety can be a reverse transcription primer which hybridizes with all mRNA species in a cell. The targeting moiety can be a primer having a specific or designed sequence which hybridizes with a particular DNA or RNA of interest.

Depending on the application and the target molecule, the targeting moiety may be a primer (which can be extended by a polymerase or a reverse transcriptase), an affinity agent that stably binds the target molecule non-covalently, or a bonding agent that forms covalent bond with the target molecule.

An affinity agent can be an oligonucleotide that binds its target by base-pairing, or an antibody, an antibody-fragment, an aptamer. Affinity agents can bind diverse types of target molecules such as proteins, nucleic acids, and particular modifications (such as phosphorylation, methylation) on proteins or nucleic acids. For example, an antibody against a specific histone methylation can be used to capture histones with such modifications and DNA physically associated with such histone.

A bonding agent may comprise ‘genus-recognizing’ functional groups such as NHS, maleimide, and Label-IT groups described above, or comprise ‘species-recognizing’ functional groups such as a suicidal substrate of a particular enzyme which form a covalent-bond of said enzyme. Examples of such ‘species-recognizing’ functional groups include halo-tag, SNAP-tag, CLIP-tag, and various probes used in activity-based protein profiling.

The immobilization moiety can associate the matrix or polymer framework through covalent bond or non-covalent interaction. The immobilization moiety can comprise (a) a chemical that is incorporated into the polymer framework, (b) a chemical or protein that stably interact with the polymer framework directly, or (c) a chemical or protein that that stably interacts with the polymer framework indirectly. An example of (a) can be methacryl group, which can be co-polymerized into polyacrylamide gel. An example of (b) can be NHS ester, which can react with primary amine group to form a stable covalent interaction. This example can be applicable if the polymer framework contains primary amine groups. Another example of (b) can be maleimide, which can react with thiol group to form a stable covalent interaction. This example can be applicable if the polymer framework contains thiol groups. Another example of (b) can be thiol group, which can react with C═C double bond-containing groups (such as maleimide and acrylate) to form a stable covalent interaction. This example can be applicable if the polymer framework contains C═C double bond-containing groups. Another example of (b) can be azide group, which can react with alkyne group to for a stable covalent interaction. This example can be applicable if the polymer framework contains alkyne group. Another example of (b) can be avidin or streptavidin, which can interact with biotin to for a stable noncovalent interaction. This example can be applicable if the polymer framework contains biotin groups. Another example of (b) can be a single-stranded DNA polynucleotide, which can interact with single-stranded polynuclotide of the reverse complementary sequence, to form a stable noncovalent interaction. This example can be applicable if the polymer framework contains single-stranded polynuclotide. An example of (c) can be biotin. This example can be applicable if the polymer contains biotin, and the hydrogel further contain streptavidin tetramer. In this case, one monomer of the streptavidin can stably interact with the biotin as the immobilization moiety and another monomer of the same streptavidin can stably interact with the biotin on the polymer. In this manner the immobilization moiety can stably interact with the polymer indirectly.

The immobilization moiety can be reacted with a reactive group on the polymer framework (e.g. hydrogel framework) through conjugation chemistry (e.g., click chemistry). As used herein, the term “reactive group” means any moiety on the monomer or polymer of the framework that is capable of reacting chemically with another reactive group on a different substance to form a covalent or ionic linkage. “Reactive group” and “functional group” can be used interchangeably. Immobilization moiety can comprise a reactive group. Examples of suitable reactive groups include electrophiles or nucleophiles that can form a covalent linkage by reaction with a corresponding nucleophile or electrophile, respectively, on the substrate of interest. Non-limiting examples of suitable electrophilic reactive groups may include, for example, esters including activated esters (e.g., succinimidyl esters), amides, acrylamides, acyl azides, acyl halides, acyl nitriles, aldehydes, ketones, alkyl halides, alkyl sulfonates, anhydrides, aryl halides, aziridines, boronates, carbodiimides, diazoalkanes, epoxides, haloacetamides, haloplatinates, halotriazines, imido esters, isocyanates, isothiocyanates, maleimides, phosphoramidites, silyl halides, sulfonate esters, sulfonyl halides, and the like. Non-limiting examples of suitable nucleophilic reactive groups may include, for example, amines, anilines, thiols, alcohols, phenols, hyrazines, hydroxylamines, carboxylic acids, glycols, heterocycles, and the like. In some cases, the reactive group is an amine, a thiol, an azide, an alkyne, a nitrone, an alkene, a tetrazine, tetrazole, or other click reactive group.

In certain cases, the interaction between the immobilizable probe and the polymer framework can be reversible so that immobilizable probe can be released from the framework if needed. For example, the immobilization moiety comprises a streptavidin and the polymer framework comprises a biotin, the interaction between the streptavidin and biotin can be reversed by adding an excess amount of free biotins. For another example, when the interaction between the immobilization moiety and the polymer framework is through nucleic acid hybridization, the interaction can be reversed by increasing the temperature to melt the nucleic acid duplex. In some cases, the immobilizable probe is linked to the polymer framework through a photo-cleavable linker.

In some cases, the immobilizable probe can be immobilized onto a diffusion restricting agent in order to entrap the immobilizable probe within the vessel or the hardened particle. The immobilization moiety can associate with the polymer framework through covalent or non-covalent interaction. In these cases, the polymer framework functions as the diffusion restricting agent. The immobilizable probe can be linked to one or more polymers and/or monomers of the framework through the immobilization moiety. The diffusion restricting agent can also be a long polymer chain directly conjugated on the immobilizable probe so that the conjugated immobilizable probe is large enough to be entangled by the polymer framework. For example, an immobilizable probe antibody can be conjugated with multiple long PEG chains so that the diffusion of the antibody is restricted within the framework. In some other cases, the diffusion restricting agent can be a large particle (e.g. micron-sized streptavidin-coated beads). The large particle is larger than the pore size of the hardened particle so that when the immobilizable probe is associated to the large particle, the immobilizable probe is entrapped within the framework of the hardened particle.

Compartmentalization

Compartmentalization of Biological Particle

The disclosure involves partitioning biological particles into compartments so that in some compartments there is only one biological particle in a compartment. In some embodiments, at least 50%, 60%, 70%, 75%, 80%, 85%, 90%, 95%, 96%, 97%, 98%, 99%, or 100% of the compartments contain zero or only one biological particle. In some embodiments, at least 50%, 60%, 70%, 75%, 80%, 85%, 90%, 95%, 96%, 97%, 98%, 99%, or 100% of the compartments contain zero or only one primer delivery particle.

The number of partitions or compartments employed can vary depending on the application. For example, the number of partitions or compartments can be about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be less than 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be about 5-10000000, 5-5000000, 5-1000000, 10-10000, 10-5000, 10-1000, 1000-6000, 1000-5000, 1000-4000, 1000-3000, or 1000-2000.

The number of biological particles (including cells and other types of biological particles) that are partitioned into compartments can be about 1, 2, 3, 4, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of cells that are partitioned into compartments can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of cells that are partitioned into compai hnents can be less than 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of cells that are partitioned into compartments can be about 5-10000000, 5-5000000, 5-1000000, 10-10000, 10-5000, 10-1000, 1000-6000, 1000-5000, 1000-4000, 1000-3000, or 1000-2000.

In some embodiments, the partition is an emulsion formed passively using a microfluidics device. These methods can involve squeezing, dripping, jetting, tip-streaming, tip-multi-breaking, or similar. Passive microfluidic droplet generation can be modulated to control the particle number, size, and diameter by altering the competing forces of two different fluids. These forces can be capillary, viscosity, and/or inertial forces upon the mixing of two solutions.

In some embodiments, the compartments are wells in a standard microwell plate with separation aided by sorting. In some embodiments, the sorter is a fluorescence activated cell sorter (FACS). Additionally, partitioning can be coupled with automated library generation in separated microfluidics chambers, as is the case with the Fluidigm Cl.

In some embodiments, the partition is a subnanoliter well and particles are sealed by a semipermeable membrane.

In some embodiments, the partition is a microfluidics droplet formed by active control of a microfluidics chip. In active control, droplet generation can be manipulated via external force application, such as electric, magnetic, or centripetal forces. A popular method for controlling active manipulation of droplets in a microfluidic chip is to modify intrinsic forces by tuning fluid velocities of two mixing solutions, such as oil and water.

Co-Partition of Other Agents

In accordance with certain aspects, the cells may be partitioned along with a lysing agent (e.g. cell lysis reagents) in order to release the contents of the cells within the partition. In such cases, the lysis agents can be contacted with the cell suspension concurrently with, or immediately prior to the introduction of the cells into the partitioning junction/droplet generation zone, e.g., through an additional channel or channels upstream of channel junction. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysing agents may additionally or alternatively be co-partitioned with the cells to cause the release of the cell's contents into the partitions. For example, in some cases, surfactant based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Similarly, lysis methods that employ other methods may be used, such as electroporation, thermal, acoustic or mechanical cellular disruption may also be used in certain cases, e.g., non-emulsion based partitioning such as encapsulation of cells that may be in addition to or in place of droplet partitioning, where any pore size of the encapsulate is sufficiently small to retain nucleic acid fragments of a desired size, following cellular disruption.

In addition to the lysing agents co-partitioned with the cells described above, other reagents can also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells, the cells may be exposed to an appropriate stimulus to release the cells or their contents from a co-partitioned microcapsule. For example, in some cases, a chemical stimulus may be co-partitioned along with an encapsulated cell to allow for the degradation of the microcapsule and release of the cell or its contents into the larger partition.

Additional reagents may also be co-partitioned with the cells, such as endonucleases to fragment the cell's DNA, DNA polymerase enzymes and dNTPs used to amplify the cell's nucleic acid fragments. Additional reagents may also include reverse transcriptase enzymes, including enzymes with terminal transferase activity, primers and oligonucleotides, and switch oligonucleotides (also referred to herein as “switch oligos”) which can be used for template switching. In some cases, template switching can be used to increase the length of a cDNA. In one example of template switching, cDNA can be generated from reverse transcription of a template, e.g., cellular mRNA, where a reverse transcriptase with terminal transferase activity can add additional nucleotides, e.g., polyC, to the cDNA that are not encoded by the template, such, as at an end of the cDNA. Switch oligos can include sequences complementary to the additional nucleotides, e.g. polyG. The additional nucleotides (e.g., polyC) on the cDNA can hybridize to the sequences complementary to the additional nucleotides (e.g., polyG) on the switch oligo, whereby the switch oligo can be used by the reverse transcriptase as template to further extend the cDNA. Switch oligos may comprise deoxyribonucleic acids, ribonucleic acids, modified nucleic acids including locked nucleic acids (LNA), or any combination.

Although the above described agents can be co-partitioned with the cells or other biological particles, it is not necessary to co-partition those agents. Since the methods described herein allow formation of hardened particles and entrapping of targets within the hardened particles, various steps can be performed after formation of hardened particles by directly mixing the agents with pooled hardened particles in a test tube. The targets originated from a single cell would still maintain their identity without mixing with the targets from a different single cell after barcoding the targets within each individual hardened particle. For example, the lysing agents can be added after pooling the hardened particles. For another example, the agents for reverse transcription can be added after pooling the hardened particles. It is to be understood that the hardened particles with entrapped targets allow for various manipulations in bulk.

In various embodiments, the biological particle can be co-partitioned with a solution comprising polymerizable or gellable polymers and/or monomers. In some embodiments, the biological particle is co-partitioned with polymerizable or gellable polymers. In some embodiments, the biological particle is co-partitioned with polymerizable or gellable monomers. In some embodiments, the biological particle is co-partitioned with a mixture of polymerizable or gellable polymers and monomers. The polymers may be of the same chemicals or different chemicals. The monomers may be of the same chemicals or of different chemicals. In some embodiments, the biological particle is co-partitioned with an immobilizable probe. In some embodiments, the biological particle is co-partitioned with a barcode-accepting polynucleotide. In some embodiments, the biological particle is co-partitioned with a solution comprising a plurality of polymerizable or gellable polymers and/or monomers, an immobilizable probe, and a barcode-accepting polynucleotide. In some embodiments, the solution further comprises agents necessary for initiating a gelation process. In some embodiments, the solution further comprises agents necessary for initiating a polymerization process of forming a gel or hardened particle.

Hardened Particle

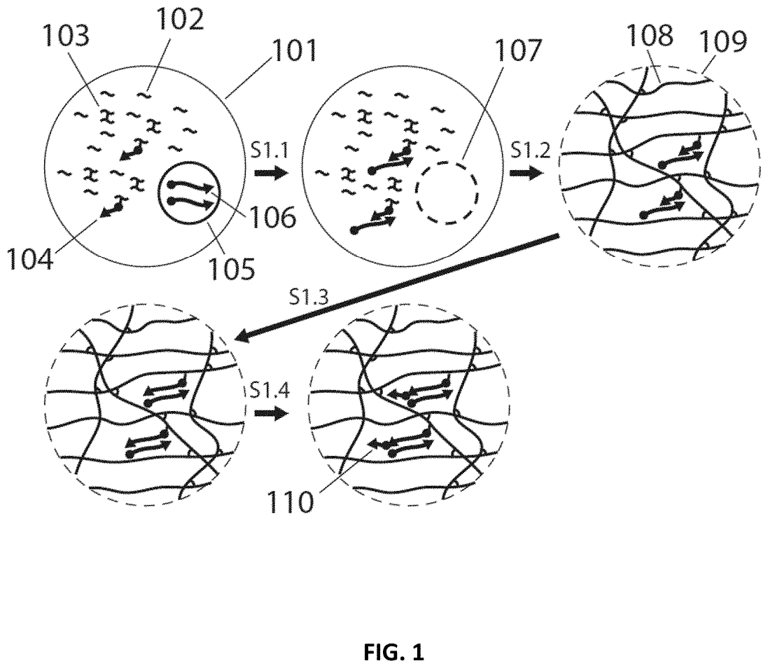

In various embodiments, the compositions or methods comprise a vessel comprising a plurality of polymerizable or gellable polymers and/or monomers. Upon polymerization or gelation of the polymers and/or monomers, the vessel can become a hardened particle comprising a polymer framework (FIG. 1).

There can be at least two advantages of the “hardened particles”. Firstly, they are dispersible in aqueous phase. This can be an important aspect because other water-soluble reagents such as enzymes, primers, barcode polynucleotides can diffuse into the hardened particle from the surrounding aqueous solution, or out of the hardened particle into the surrounding aqueous solution. This property distinguishes hardened particles from the water-in-oil droplets which are not dispersible in aqueous phase (i.e., they are only dispersible in oil phase). Secondly, they are non-mergable. This can be another important aspect because one needs to minimize the targets from different hardened particles from mixing. This property distinguishes hardened particles from droplets of highly viscous liquid (e.g., glycerol) which may merge with each other in routine laboratory handling. Because of the advantages, many steps can be performed after polymerizing or gelling of the polymers and/or monomers. For example, lysing entrapped cells, barcoding, washing, or primer extension (e.g. reverse transcription) can be performed after obtaining hardened particles. It is to be understood that these steps can also be performed before polymerizing or gelling.

In one aspect, the present disclosure provides a hardened particle comprising: a polymerized or gelled plurality of polymers and/or monomers; a single cell, a single complex of cells, or a single exosome; an immobilizable probe linked to one or more polymers and/or monomers of the polymerized or gelled plurality; and a barcode-accepting polynucleotide. In some embodiments, the immobilizable probe comprises an immobilization moiety. In some embodiments, the immobilization moiety links the immobilizable probe to one or more polymers and/or monomers of the polymerized or gelled plurality. In some embodiments, the immobilization moiety is a chemical that is co-polymerized with the polymers and/or monomers, a chemical or protein that stably interact with the polymers and/or monomers directly, or a chemical or protein that stably interacts with the polymers and/or monomers indirectly. In some embodiments, the immobilization moiety is an NHS ester, a biotin, a maleimide group, a thiol group, an azide group, an avidin or streptavidin, a single-stranded polynucleotide, a biotin group, or a methacryl group.

In another aspect, the present disclosure provides a hardened particle comprising: a polymerized or gelled plurality of polymers and/or monomers; a single cell, a single complex of cells, or a single exosome; an immobilizable probe, wherein the immobilizable probe has a hydrodynamic radius that is larger than a pore size of the vessel; and a barcode-accepting polynucleotide. In some embodiments, the immobilizable probe comprises an immobilization moiety. In some embodiments, the immobilization moiety comprises an immobilization polymer. In some embodiments, the immobilization polymer is a polyethylene glycol molecule.

In various embodiments, the hardened particle further comprises a target from the single cell, the single complex of cell, or the single exosome. In some embodiments, the target is bound to the immobilizable probe. In some embodiments, the target is a target polynucleotide, a target polypeptide, a target organelle, or any combination thereof. In some embodiments, the target polynucleotide is a target deoxyribonucleic acid (DNA), a target ribonucleic acid (RNA), or a combination thereof. In some embodiments, the target DNA is a modified DNA, for example, a methylated DNA. In some embodiments, the target RNA is total RNA within a cell, a messenger RNA, a non-coding RNA, a microRNA, a ribosomal RNA, a piwi-RNA, a circular RNA, or any combination thereof.

In some embodiments, the hardened particle further comprises a complementary strand of the target polynucleotide. For example, when the target is an mRNA, the immobilizable probe comprising a reverse transcription primer can bind to the mRNA and the reverse transcription can further be extended to synthesize the complementary cDNA strand of the mRNA target. In some embodiments, the complementary strand is a cDNA strand. In some embodiments, the target organelle is a target mitochondria or a target nucleus.

The immobilizable probe within the hardened particle can be responsible for linking the target into the polymer framework. In some embodiments, the immobilizable probe comprises a targeting moiety. In some embodiments, the targeting moiety is a polynucleotide, a polypeptide, or a chemical group. In some embodiments, the polynucleotide is a primer or an oligonucleotide aptamer. In some embodiments, the primer is a reverse transcription primer. In some embodiments, the reverse transcription primer comprises a poly-deoxy-thymidine nucleotide sequence. In some embodiments, the polypeptide is an antibody or a fragment thereof, or a peptide aptamer. In some embodiments, the chemical group is a reactive group. Non-limiting examples of suitable electrophilic reactive groups may include, for example, esters including activated esters (such as, for example, succinimidyl esters), amides, acrylamides, acyl azides, acyl halides, acyl nitriles, aldehydes, ketones, alkyl halides, alkyl sulfonates, anhydrides, aryl halides, aziridines, boronates, carbodiimides, diazoalkanes, epoxides, haloacetamides, haloplatinates, halotriazines, imido esters, isocyanates, isothiocyanates, maleimides, phosphoramidites, silyl halides, sulfonate esters, sulfonyl halides, and the like. Non-limiting examples of suitable nucleophilic reactive groups may include, for example, amines, anilines, thiols, alcohols, phenols, hyrazines, hydroxylamines, carboxylic acids, glycols, heterocycles, and the like. In some embodiments, the reactive group forms a covalent bond with an amino acid side chain or a nucleobase of the target. In some embodiments, the reactive group is an NHS ester, a maleimide group, or Label-IT linker and reactive group. In some embodiments, the amino acid side chain is lysine or cysteine side chain. In some embodiments, the nucleobase is guanine.

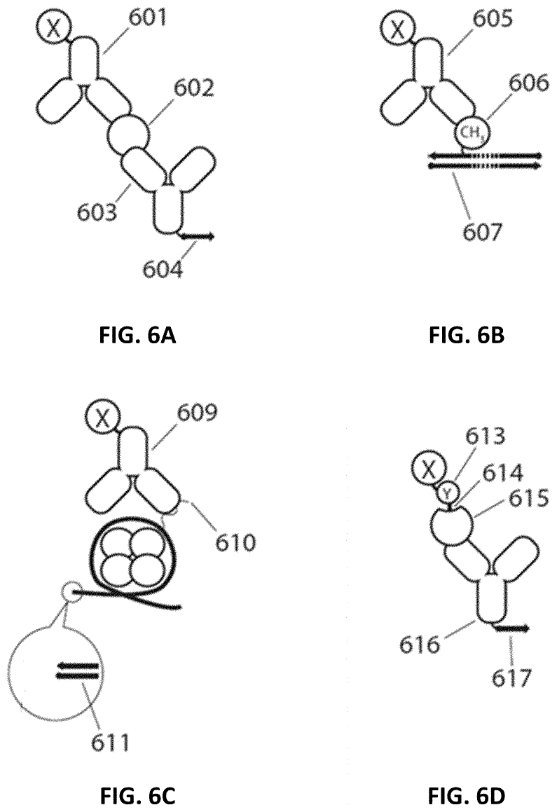

In some embodiments, the hardened particle further comprises a barcode. The barcode is attached to the barcode-accepting polynucleotide. The barcode-accepting polynucleotide can be a 5′ or 3′ segment of the target polynucleotide, a complementary strand of the target polynucleotide, a polynucleotide strand attached to the immobilizable probe, or a polynucleotide strand attached to a target probe that binds to an immobilized target. For example, FIGS. 4C-D and FIGS. 6A-D illustrate several situations where the barcode-accepting polynucleotide can be attached to. In some embodiments, the barcode is attached to the immobilizable probe or the target. In some embodiments, the barcode is from 5 to 500 nucleotides in length. In some embodiments, the polymers are polysaccharides or polyacrylamides. In some embodiments, the polysaccharides are poly-agarobioses or alginate. In some embodiments, the monomers are acrylamide or methacrylamide monomers. In some embodiments, the polymerized or gelled plurality of polymers and/or monomers are cross-linked.

In some embodiments, a vessel or compartment comprising a plurality of polymerizable or gellable polymers and/or monomer is generated. The vessel or compartment can be a droplet, for example, a water-in-oil droplet in an emulsion. In the cases where the vessel is a water-in-oil droplet, after polymerizing or gelling the polymers and/or monomers, the hardened particle may be surrounded by a thin liquid layer (e.g. water phase). Agents that can break the emulsion may be useful to isolate the hardened particle for any downstream manipulations.

Any gellable solution or gel-forming agent may be used in the present disclosure. The gellable solution or gel forming agent can be compartmentalized into one or more vessels, and gelation process can be triggered in each of the one or more vessels resulting in hardened particle formation. The gellable solution or gel-forming agent can comprise gellable polymers or monomers. The gellable polymers or monomers can be polymerizable polymers or monomers. In some cases, the hardened particle is a hydrogel particle.

A crosslinked polymer can be formed by reacting or contacting proper proportions of a crosslinkable polymer with a crosslinking agent. The gel-forming composition may at least contain either the crosslinkable polymer or the crosslinking agent. When the crosslinkable polymer or crosslinking agent is omitted from the composition, the omitted material can be introduced into the composition, either before, after, or simultaneously with the introduction of the gel-forming composition. In some embodiments, the composition comprises at least the crosslinkable polymer or monomers capable of polymerizing to form a crosslinkable polymer (e.g., acrylamide, vinyl acetate, acrylic acid, vinyl alcohol, and methacrylamide). In some embodiments, the composition comprises both (a) the crosslinking agent and (b) either (i) the crosslinkable polymer or (ii) the polymerizable monomers capable of forming a crosslinkable polymer.

The crosslinkable polymer can be water soluble. Common classes of water soluble crosslinkable polymers include polyvinyl polymers, polymethacrylamides, cellulose ethers, polysaccharides, lignosulfonates, ammonium salts thereof, alkali metal salts thereof, as well as alkaline earth salts of lignosulfonates. Examples of water soluble polymers include, but are not limited to, acrylic acid-acrylamide copolymers, acrylic acid-methacrylamide copolymers, polyacrylamides, partially hydrolyzed polyacrylamides, partially hydrolyzed polymethacrylamides, polyvinyl alcohol, polyvinyl acetate, carboxymethylcelluloses, carboxyalkylhydroxyethyl celluloses, hydroxyethylcellulose, galactomannans (e.g., guar gum), substituted galactomannans (e.g., hydroxypropyl guar), heteropolysaccharides obtained by the fermentation of starch-derived sugar (e.g., xanthan gum), and ammonium and alkali metal salts thereof. Additional water soluble crosslinkable polymers can include hydroxypropyl guar, partially hydrolyzed polyacrylamides, xanthan gum, polyvinyl alcohol, and the ammonium and alkali metal salts thereof.

The crosslinking agents can be selected from the group consisting of aldehydes, hexamethylenetetramine (HMT), and mixtures thereof. Examples of aldehydes include, but are not limited to, formaldehyde, paraformaldehyde, acetaldehyde, propionaldehyde, butyraldehyde, isobutyraldehyde, valeraldehyde, heptaldehyde, glutaraldehyde, and terephthaldehyde.

The acrylamide polymer may be a linear or crosslinked polyacrylamide or a mixture thereof, or water-dispersible copolymer resulting from the polymerization of a major proportion of acrylamide and a minor proportion of an ethylenically unsaturated monomer copolymerizable therewith, e.g., a copolymer of from 90% to 99% acrylamide and from 1% to 10% of a monomer selected from the class consisting of acrylic acid, methacrylic acid, vinylsulfonic acid, vinylbenzylsulfonic acid, vinylbenzenesulfonic acid, and alkali and alkaline earth metal salts of such acids.

The hardened particles comprising one or more targets can be labeled with a detectable label. Examples of labels include chromophores, phosphors, or fluorescent dyes. Suitable dyes for use include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue™ Texas Red, alexa dyes, phycoerythin, and bodipy. Examples of nucleic acid dye includes, but are not limited to, SYBR™ GREEN I, ethidium bromide, pico green, acridine orange, thiazole orange, and chromomycin A3. Additional examples of nucleic acid binding dyes include, PO-PRO™-1, BO-PRO™-1, SYTO® 43, SYTO® 44, SYTO® 45, SYTOX® Blue, POPO™-1, POPO™-3, BOBO™-1, BOBO™-3, LO-PRO™-1, JO-PRO™-1, YO-PRO®-1, TO-PRO®-1, SYTO® 11, SYTO® 13, SYTO® 15, SYTO® 16, SYTO® 20, SYTO® 23, TOTO™-3, YOYO®-3 (Molecular Probes, Inc., Eugene, Oreg.), GelStar® (Cambrex Bio Science Rockland Inc., Rockland, Me.), thiazole orange (Aldrich Chemical Co., Milwaukee, Wis.), EvaGreen, PicoGreen, Cyto 9, LC Green, SYBR GreenER, Ethidium bromide, TOTO, YOYO, Bebo, SYTO Green, bexto, SYBR Gold, SYBR Safe.

Polymer Framework Formation

The gellable solution or gel forming agent can be compai linentalized into one or more vessels, and gelation process can be triggered in each of the one or more vessels resulting in hardened particle formation. The gellable solution or gel-forming agent can comprise gellable polymers or monomers. The gellable polymers or monomers can be polymerizable polymers or monomers.

In some cases, polymer hydrogels are used. Examples of hydrogels include both natural—(e.g., alginate (ALG), chitosan, hyaluronic acid (HA), gelatin, heparin (HEP), collagen, fibrin, dextran (DEX), etc.) and synthetic—(e.g., polyesters, polyethyleneimine (PEI), etc.) based polymers. They are formed by various mechanisms, including self-assembly of polymers and peptides, chemical crosslinking, ionic crosslinking and biomolecule recognition.

An example of a gel or hardened matrix formed by polymerization mechanism includes, but is not limited to, polyacrylamide. Polyacrylamide can be a matrix formed from monomers of acrylamide and bis-acrylamide. The polymerization reaction can be a vinyl addition catalyzed by free radicals. The reaction can be initiated by TEMED, which induces free radical formation from ammonium persulphate (APS). The free radicals can transfer electrons to the acrylamide/bisacrylamide monomers, radicalizing them and causing them to react with each other to form the polyacrylamide chain. In the absence of bis-acrylamide, the acrylamide would polymerize into long strands, not a porous gel. Bis-acrylamide can cross-link the acrylamide chains, giving rise to the formation of the porous gel matrix. The amount of crosslinking, and therefore the pore size and consequent separation properties of the gel can be controlled by varying the ratio of acrylamide to bis-acrylamide. An example of a gel or a hardened matrix formed by a non-polymerization mechanism includes, but is not limited to, an agarose gel. Agarose is a polysaccharide, whose monomeric unit is a disaccharide of D-galactose and 3,6-anhydro-L-galactopyranose. In aqueous solutions below 35° C. these polymer strands can be held together in a porous gel structure by non-covalent interactions like hydrogen bonds and electrostatic interactions. Heating the solution can break these non-covalent interactions and separates the strands. Then as the solution cools, these non-covalent interactions can be re-established and the gel can form. Therefore, agarose gels can form by gelation through hydrogen bonding and electrostatic interactions. Additional materials that may be used for hydrogel particle formation can be found in Minh Khanh Nguyena and Eben Alsberg, Prog Polym Sci. 2014 July; 39(7): 1236-1265, which is entirely incorporated herein by reference.

Self-Assembly of Polymers in Response to Temperature

Self-assembly of polymers can be used to prepare physically crosslinked hydrogels. Self-assembly occurs with some polymers as a result of intra- and intermolecular forces, such as hydrogen bonding and hydrophobic interactions. Aqueous solutions of these polymers can undergo sol-to-gel transition upon self-assembling in response to external stimuli such as pH and temperature.

Self-assembly of thermo-responsive (or thermogelling) polymers can be used to fabricate hydrogels by a change in temperature. Temperature-sensitive polymers can be synthesized by post-polymerization grafting of a hydrophobic block to a hydrophilic block or by co-polymerization to create amphiphilic diblock (AB), triblock (ABA or BAB type) or multiblock copolymers. A is a hydrophilic block like PEG (also known as poly(ethylene oxide) (PEO)) while B is a hydrophobic block such as a polyester, poly(propylene oxide) (PPO) (also called poly(propylene glycol) (PPG)), or poly(N-isopropylacrylamide) (PNIPAm). The amphiphilic block copolymers can self-assemble in water to form micelles with shells of hydrophilic blocks and cores of hydrophobic blocks at low temperatures, and association of the micelles at elevated temperatures triggers gelation. The temperature at which a thermo-responsive polymer solution changes to a gel is called the gelation temperature. Poloxamer (ABA type PEO—PPO-PEO polymer), known as Pluronic® (BSAF) or Synperonic® PE (ICI), can be used to form thermo-sensitive hydrogels. Aliphatic esters, like poly(ε-caprolactone) (PCL) and poly(lactic acid) (PLA), can be coupled to the ends of Pluronic® via ring opening polymerization (ROP) of corresponding ε-caprolactone (CL) and lactic acid (LA) monomers using stannous octoate (Sn(oct)₂) as a catalyst to prepare hydrolytically degradable Pluronic® hydrogels.

Thermo-sensitive hydrogels based on PEG with aliphatic esters such as PLA, PCL, poly(glycolic acid) (PGA), and poly[(R)-3-hydroxybutyrate] (PHB) can be used. For example, ABA type PEG-poly(D,L-lactide-co-glycolide)-PEG (PEG-PLGA-PEG) triblock copolymers can form hydrolytically degradable hydrogels. Gelation and degradation of these hydrogels can be tailored by varying the molecular weight of the hydrophobic and hydrophilic blocks, the composition of the hydrophobic blocks, polymer concentration and additives. Gelation behavior of PLGA-PEG-PLGA can be modulated by incorporation of various end groups (i.e., hydroxyl, acetyl, propionyl, and butanoyl groups).

PNIPAm can be soluble in aqueous solution at room temperature but precipitates above 32° C. (phase transition temperature) due to its coil-to-globule transition. Incorporation of PNIPAm with other polymers can result in copolymers that exhibit sol-to-gel phase transition in aqueous solution in response to increased temperature. Radical polymerization can be used to incorporate NIPAm with other methacrylate or acrylate monomers/polymers to create PNIPAm-based polymers. For example, a PNIPAm-poly(2-metha-cryloyloxyethyl phosphorylcholine)-PNIPAm (PNIPAm-MPC-PNIPAm) copolymer can be synthesized via atom transfer radical polymerization (ATRP). The polymer solution can form a gel as temperature is raised above 32° C. due to hydrophobic interactions between the polymer chains during the formation of a network. Thermo-responsive polyphosphazenes can also display sol-to-gel phase transition in aqueous solutions with increasing temperature.

An ABA-type triblock copolymer consisting of MPEG and poly(propylene fumarate) (PPF) can result in a thermo-sensitive gel that can be further stabilized through crosslinking of unsaturated double bonds on PPF.

Mixing of enantiomeric PEG-P(L-lactide)-PEG (PEG-PLLA-PEG) and PEG-P(D-lactide)-PEG (PEG-PDLA-PEG) triblock copolymers can induce sol-to-gel transition. Hydrogels can be formed when temperature is increased to 37° C. and they can become solutions above 70° C. Similarly, hydrogels can be formed by stereocomplexation of enantiomeric PEG-(PLLA)8 and PEG-(PDLA)8 star block, and PEG-(PLLA)2 and PEG-(PDLA)2 triblock copolymers.

In addition to self-assembly of synthetic polymers, natural materials chemically modified with synthetic molecules can also self-assemble in aqueous media to form hydrogels. For example, chitosan, a polysaccharide derived from the partial deacetylation of naturally abundant chitin, can be used for hydrogel formation. Chitosan can form physical hydrogels when conjugated with several polymers. For instance, PEG-aldehyde can be coupled to chitosan via Schiff's base reaction followed by reduction with sodium cyanoborohydride (NaBH₃CN) to yield PEG-g-chitosan. The resulting graft polymer is a solution at low temperatures and can transform to a gel at around 37° C. temperature. The gelation can be attributed to hydrophobic interactions between the polymer chains, which lead to association of chitosan segments and a decrease in PEG mobility. Similarly, Pluronic®-g-chitosan also exhibits thermo-reversible sol-to-gel transition upon heating.

Self-Assembly of Polymers in Response to PH and Temperature

Some polymers bearing cationic or anionic groups can exhibit sol-to-gel transition in response to both pH and temperature. A poly(amidoamine)-PEG-poly(amidoamine) (PAA-PEG-PAA) copolymer can be synthesized by the addition of 4,4-trimethylene dipiperidine (TMDP) and 1,10-decylene diacrylamide to amine-functionalized PEG via Michael-type step polymerization. The PAA block is hydrophilic at low pH, but it can become more hydrophobic at a higher pH and/or temperature because of deprotonation of the tertiary amine groups on PAA. Micelles can be formed as the hydrophobicity of PAA increased and aggregation of the micelles at higher pH and/or temperature can lead to gelation. Unlike polymers bearing amine groups, a triblock copolymer with a central PEO block flanked by poly(methoxydi(ethylene glycol) methacrylate-co-methacrylic acid) (P(DEGMMA-co-MAA)-b-PEO-b-P(DEGMMA-co-MAA)) synthesized through ATRP can gel at acidic pH as a result of the carboxylic acid groups. In addition, synthesized multiblock poly(amino urea urethane) (PAUU) composed of PEG, 2-hydroxyethyl piperazine and HDI can form a gel in response to pH and temperature.

Another example of pH- and temperature-sensitive copolymer hydrogels is based on anionic oligomer sulfamethazine (OSM). A thermo-sensitive triblock copolymer of poly(CL-co-LA)-PEG-poly(CL-co-LA) (PCLA-PEG-PCLA) can be synthesized via ROP of CL and LA monomers on PEG; subsequently, the OSM can be conjugated to the end groups of the copolymer using 4-(dimethylamino)pyridine (DMAP)/N,N′-dicyclohexylcarbodiimide (DCC) chemistry. At high pH (e.g., 8.0), the OSM blocks can be ionized and the block copolymer becomes a free flowing sol. Deionization of the OSM at lower pH (e.g., 7.4) can result in sol-to-gel transition with increasing temperature. The gel window can be adjusted by varying the molecular weights of OSM, PEG and the PCLA blocks as well as the polymer concentration. Similarly, cationic poly(β-aminoester)s (PAE)s can be extended to an acrylate-activated PCL-PEG-PCL copolymer via Michael addition polymerization using 1,4-butandiol diacrylate (BDA) and TMDP. The polymers underwent sol-to-gel transition at basic pH but are solutions at acidic pH. Dual responsive amphiphilic block copolymers based on poly(amino ester urethane) (PAEU) or PAE with a central PEG block can be synthesized via Michael addition polymerization, which formed gels at about 37° C. temperature. These polymers are constructed such that their pH-sensitive segments can hydrolyze in aqueous media, without conjugating to hydrolysable temperature sensitive polymers. Cationic chitosan can be used to prepare pH- and temperature-sensitive hydrogels. Palmitic acid N-hydroxysuccinimide (PANHS) ester can be grafted to chitosan to produce a biodegradable and biocompatible pH-triggered N-palmitoyl-chitosan hydrogel in the pH range of 6.5-7.0. The gelation process may involve a balance between charge repulsion and hydrophobic interactions.

Self-Assembly of Polymers Based on Inclusion Complexes

Hydrogels can be formed based on inclusion complexes between polymers and cyclodextrins (CDs). CDs are cyclic oligosaccharides composed of 6, 7 or 8 D(+)-glucose units circled by D(+)-1,4-bonds (called α-, β- and γ-CD, respectively) with a hydrophobic inner cavity and a hydrophilic exterior. Several molecules can penetrate to the cavity of CDs to form inclusion complexes with necklace-like supramolecular structures, which can be used as a crosslinking method for preparation of supramolecular hydrogels. For example, a linear water-soluble polymer such as high molecular weight PEO can be inserted into the pocket of α-CD to induce inclusion complexes. The supramolecular self-assembly of the inclusion complexes in water can induce physically crosslinked hydrogels. To prepare more stable hydrogels, triblock copolymers composed of a central PPO flanked with PEO blocks can be used to form inclusion complexes with α-CD. PEO—PHB-PEO can also gel in the presence of α-CD due to the cooperative effect of complexation of PEO with α-CD and hydrophobic interactions between PHB blocks. The use of the polymers with the central hydrophobic blocks can result in more stable hydrogels. β-CD/cholesterol inclusion complexes driven by hydrophobic and van der Waals interactions can induce thermo-reversible hydrogels when β-CD and cholesterol end-capped 8-arm PEG solutions are mixed. The hydrogel properties are a function of the polymer concentration, α-CD/cholesterol stoichiometry, molecular weight of PEG and PEG architecture.

Self-Assembly of Peptides

Self-assembly of peptides and proteins can be an approach for constructing hydrogels. Peptides and proteins can have α-helix, β-sheet, and random coil structures under specific conditions and their building blocks can be prepared with controllable hydrophobic, hydrophilic and ionic characteristics. Their self-assembly is highly specific among the peptide building blocks, sensitive to external stimuli (such as pH or temperature discussed in the previous sections, or ionic strength) and reversible. For example, an ERK 16 peptide (Ala-Glu-Ala-Glu-Ala-Lys-Ala-Lys)2 (SEQ ID NO: 2) displays β-sheet structure in water, and it can assemble to form a stable macroscopic hydrogel membrane in PBS. The complementary ionic bridges between negatively charged Glu and positively charged Lys on one side of the β-sheet and hydrophobic interactions between Ala molecules on the other side can result in gelation. For another example, peptide-grafted copolymers can gel through the formation of antiparallel heterodimeric coiled-coils. Copolymers of MPEG-poly(L-glutamate)s with different hydrophobic side groups, such as methyl, ethyl, n-propyl and n-butyl can be synthesized via ROP and they can undergo sol-to-gel transition when temperature increases. The partial dehydration of MPEG and β-sheet formation in the polypeptides can lead to the gelation.

Chemically Crosslinked Hydrogels

Chemically crosslinked networks can be constructed through chemical reactions, for example: Schiff's base, enzyme-mediated, click, photopolymerization, Michael-type addition, and disulfide formation reactions. The chemical crosslinking methods can result in hydrogels with controllable crosslink density and network properties.

Chemically Crosslinked Hydrogels: Photocrosslinked Hydrogels

Photocrosslinked hydrogels can be formed under short exposure to visible or ultraviolet (UV) light in the presence of light-sensitive compounds (called photoinitiators). UV light can be used to decompose photoinitiators to generate free radicals which initiate methacrylate or acrylate end-capped macromers to produce photocrosslinked hydrogels. Photopolymerization strategies can provide spatiotemporal control over network formation and fast gelation kinetics (seconds to minutes) at room or physiological temperatures, and produce minimal heat, which can be used for encapsulation of biological particles or molecules. The hydrogels can be formed by partitioning the photopolymerizable macromer solutions into individual vessels and the gels are then formed by applying UV light externally.

An example of a photopolymerizable hydrogel consisting of a synthetic poly(α-hydroxy acid)-PEG-poly(α-hydroxy acid) triblock copolymer terminated with acrylate groups. An acrylated macromer solution with 2,2-dimethoxy-2-phenylacetophenone (Irgacure 651) photoinitiator or a mixture of ethyl eosin and triethanolamine can form a gel under exposure to UV light or visible light, respectively. In addition, PEG-diacrylate (PEGDA) can form hydrogels in the presence of Irgacure 651 photoinitiator. Another example of photopolymerizable hydrogel includes photopolymerizable thermo-sensitive triblock copolymers composed of a central PEG block extended with partly methacrylated poly(HPMA lactate) (PHPMAlac) blocks at two ends, which can form hydrogels under exposure to UV light.

Methacrylated HA conjugates can be synthesized by coupling synthetic GMA or methacrylic anhydride (MA) to natural HA, and then they can be exposed to UV light in the presence of 4-(2-hydroxyethoxy)phenyl-(2-hydroxy-2-propyl)ketone (Irgacure 2959) photoinitiator to fabricate cytocompatible hydrogels. Methacrylated ALG (ALG-MA) can also be used. The material can be produced using EDC chemistry by conjugating 2-aminoethyl methacrylate (AEMA) to carboxylic acid groups of ALG in the presence of 1-ethyl-3-(3-dimethylaminopropyl)-carbodiimide hydrochloride (EDC) and N-hydroxysuccinimide (NHS). Similarly, water-soluble natural carboxymethylcellulose (CMC) can be coupled with AEMA via EDC chemistry to achieve CMC-AEMA macromers. The macromers can be photocrosslinked with PEG-dimethacrylate (PEGDM) to form cytocompatible and degradable hydrogels.

Thiol-ene photopolymerization based on a reaction of a thiol and a vinyl group in the presence of a photoinitiator can be used to prepare hydrogels. This type of hydrogel can be based on 4-arm-PEG, which can be reacted with 5-norbornene-2-carboxylic acid (NORB) to form 4-arm-PEG-NORB. Subsequently, the NORB-terminated PEG solution containing Irgacure 2959 can crosslink with dithiol-functionalized chymotrypsin-degradable peptides under exposure to UV light to result in hydrogel formation.

Chemically Crosslinked Hydrogels: Michael Addition

Michael reaction between thiol groups and methacrylate/acrylate or vinyl groups can occur at physiological conditions without the addition of chemicals or catalysts or interaction with amine groups of proteins. The reaction can allow macromer solutions to gel in contact with living tissue, biologic molecules and cells without crosslinking to them. This type of hydrogel can be prepared by mixing PEG-acrylates with PEG-dithiol at 1:1 stoichiometry and physiological pH. A simultaneously physically and chemically gelling polymer system consisting of poly(NIPAm-co-cysteamine) and PEGDA is another example. The physically and chemically crosslinked material can display improved mechanical properties compared to the poly(NIPAm-co-cysteamine) physical gels alone.

Modified natural and synthetic polymers can be combined to form hydrogels via Michael-type addition. Dithiobis (propanoicdihydrazide) (DTP) can be reacted with HA via EDC chemistry followed by reducing their initial disulfide bonds using dithiothreitol (DTT) to obtain thiolated HA (i.e., HA-DTP). In another example, thiolated-DEX (DEX-SH) can crosslink with PEG-tetracrylate or DEX-vinyl sulfone (DEX-VS) to create hydrogels. Hydrogels based on thermo-sensitive triblock copolymers with HA-SH can also be used. For another example, a cysteine-containing matrix metalloproteinase (MMP) sensitive peptide susceptible to proteolytic degradation can be used to form hydrogels via Michael reaction with multi-arm PEG-VS, and these resultant hydrogels can be broken down proteolytically.

Chemically Crosslinked Hydrogels: Disulfide Formation

Polymers with thiol groups, or thiomers, can form inter- and/or intramolecular disulfide bonds at pH above 5 in the absence of toxic crosslinkers to yield hydrogels. Disulfide crosslinked hydrogels form via oxidation of thiol groups in air or oxidizing agents. While air drives disulfide formation over a time period of several minutes to hours, oxidizing agents (i.e., H₂O₂, sodium periodate (NaIO₄), ammonium persulfate ((NH₄)₂S₂O₈), sodium hypochlorite (NaOCl) and sodium borohydride (NaBH₄)) can reduce the reaction time. Reversible hydrogels can be formed from thiolated HA through a disulfide crosslinking strategy. HA-dithiobis(butyric dihydrazide) (HA-DTB) can be prepared in the same way to the synthesis of HA-DTP. The number of disulfide linkages formed can be increased when the thiol groups are oxidized by hydrogen peroxide (H₂O₂) rather than air. HA-DTP can display faster gelation when in contact with air compared to HA-DTB, due to the lower pKₐ of thiols in HA-DTP. Similarly, gelatin can be modified with DTP using EDC chemistry and the resulting gelatin-DTP can crosslink with HA-DTB to form hydrogels. 8-arm-PEG-SH can form gels through intra- and/or intermolecular disulfide bridges in the presence of H₂O₂. In contrast, hydrogels can form from the exclusively intermolecular disulfide formation between 8-arm-PEG-SH and 8-arm-PEG-S-thiopyridyl (8-arm-PEGS-TP) at pH 8.0 without oxidation.

Chemically Crosslinked Hydrogels: Schiff's Base Reaction

Schiff's base crosslinking reaction is a condensation of aldehyde with amine groups without use of any chemicals or catalysts. Therefore, macromers with aldehyde groups can react with those containing amine groups to form hydrogels. Polysaccharides are often oxidized by the reaction with NaIO₄ to create aldehyde groups on their main chain. Gelation reaction of aldehyde-modified DEX (DEX-CHO) and amino groups in gelatin can be used for the formation of hydrogels. The gelation rate can dependent on macromer solution pH and ionic strength, and gel strength can increase with the degree of DEX oxidation. DEX-CHO or oxidized carboxymethylcellulose (CMC-CHO) can also copolymerize with hydrazide-modified carboxymethyldextran (CMDX-ADH) to form hydrogels. In addition, oxidized HA (HA-CHO) can be crosslinked with N-succinyl-chitosan (S-chitosan) via Schiff's base reaction for preparation of hydrogels. Chitosan may have poor solubility in water under pH 7-7.5; therefore, the succinyl groups may be introduced at the N-position of the glucosamine units to enhance its solubility.

Chemically Crosslinked Hydrogels: Enzyme-Crosslinked

Enzymatic reactions can be used to prepare chemically crosslinked hydrogels because they exhibit high specificity and can occur in aqueous media without unwanted side reactions. Some solutions of modified macromers can be crosslinked by addition of horseradish peroxidase (HRP) and H₂O₂ to form hydrogels. HRP is a single-chain b-type hemoprotein that acts as a catalyst for inducing the oxidative coupling of phenol moieties in the presence of H₂O₂ as an oxidant. The oxidative reaction proceeds at C—C and C—O sites between the phenols under mild conditions. Different materials such as chitosan, DEX, CMC, ALG, PEG/CD and gelatin/4-arm PPO-PEO can be used to prepare chemically crosslinked hydrogels using peroxidases.

Transglutaminases (TGase) catalyze the formation of covalent γ-glutamyl-ε-lysine bonds between the γ-carboxamide group of a peptide-bound glutamine residue and ε-amino group of a peptide bound lysine or the primary amino group of polyamine in a calcium-dependent reaction. TGase can be used to catalyze formation of hydrogels. For example, fibrin hydrogels can be formed by covalently crosslinking bi-domain peptides during fibrinogen polymerization to fibrin through the action of TGase factor XIIIa. PEG-peptide conjugates containing glutamine and lysine residues can form hydrogels in the presence of TGase. Additionally, cytocompatible TGase-catalyzed hydrogels can be prepared via the crosslinking of genetically engineered protein polymers serving as lysine substrates with random coil glutamine containing proteins. Changing compositions of precursors can regulate gel strength, swelling properties and microstructure of the hydrogels. In addition to HRP/H₂O₂ and TGase enzymes, other enzymes, such as tyrosinase, phosphopantetheinyl transferase, an acid phosphatase, thermolysin, lysyl oxidase and an esterase, can be used to catalyze hydrogel formation.

Chemically Crosslinked Hydrogels: Click Reaction

Click chemistry can be used as a crosslinking strategy for hydrogel formation. One example of click chemistry is a Huisgen cycloaddition reaction between an organic azide and an alkyne. The reaction can be fast and complete when copper (I) (Cu(I)) is used as a catalyst. An example of “click reaction”-based hydrogels is to use poly(vinyl alcohol) (PVA). PVA can be functionalized with 1-azido-2-aminoethane and propargylamine (or N-methylpropargylamine) to prepare azide- and alkyl-modified PVAs, respectively. Upon simple mixing of the PVA macromers in the presence of Cu(I) and sodium ascorbate in water, the hydrogels formed via chemoselective 1,3-cycloaddition between azido and alkynyl functional groups of PVA. The properties of these hydrogels can be dependent on the polymer structure and concentration, stoichiometry, and catalyst concentration. This chemistry can be used to prepare PEG, HA, guar, and thermo-sensitive p(NIPAm-co-HEMA)/cellulose gels. A Cu(I)-free azide-alkyne click reaction may be used for the preparation of hydrogels. In addition, hydrogels can also be formed via the Cu(I)-free Diels-Alder reaction; similar to the Cu(I)-free azide-alkyne system, hydrogel precursor solutions may require long incubation times for gelation.

Chemically Crosslinked Hydrogels: Other Methods

Genipin, a natural product, isolated from gardenia fruit can crosslink functional amine groups of macromers to form biocompatible hydrogels. The genipin crosslinker can be employed to fabricate hydrogels based on polymers containing primary amine groups, such as amine-terminated PEG, gelatin, and chitosan.

Redox reactions that use ammonium persulfate/N,N,N′,N′-tetramethylethylene diamine (APS/TEMED) or APS/ascorbic acid can produce free radicals in aqueous solutions for polymerization of methacrylate/acrylate-modified macromers. Hydrogels based on DEX and PEG methacrylate, PEGLADA, and LA-Pluronic®-LA acrylate, poly(propylene fumarate-co-ethylene glycol) [P(PF-co-EG)] acrylate and acrylate functionalized ortho-nitrobenzylether (o-NBE)-PEG-o-NBE can be formed via redox reactions.

Ionically Crosslinked Hydrogels

Several ions, such as divalent cations (e.g., Ca²⁺) and anions (e.g., β-glycerophosphate (BGP)), can result in reversible hydrogels upon formation of ionic bridges between the ions and some biomaterials (e.g., chitosan, ALG). BGP was slowly added to a chitosan solution to obtain a clear liquid solution with pH of 7.15. The BGP/chitosan solution can become a gel when exposed to body temperature and the gelation temperature increased with decreasing degree of deacetylation of chitosan. The addition of BGP to the chitosan solution can affect electrostatic and hydrophobic interactions and hydrogen bonding between chitosan chains, which can drive gel formation. One of the methods to crosslink ALG is the use of divalent cation crosslinking agents (e.g., Ca²⁺, Ba²⁺, etc.). The crosslinking can occur between the divalent cations and the carboxylic acid groups in α-guluronic acid (G) of ALG, resulting in an egg-box structure, and the divalent cations need to crosslink with a minimum 20 G in a row for ALG hydrogels to form. Calcium sulfate (CaSO₄), calcium chloride (CaCl₂) and calcium carbonate (CaCO₃) are examples of some of the chemicals that can be used to ionically crosslink ALG with calcium. Reaction of ALG with highly soluble CaCl₂ may result in hydrogels with nonuniform structure. In contrast, CaSO₄ and CaCO₃ can allow for crosslinking at a slower rate because of their lower solubility. ALG can be crosslinked with CaCO₃ in the presence of D-glucono-d-lactone to form hydrogels with structural uniformity and controllable, consistent mechanical properties. The addition of D-glucono-d-lactone can trigger liberation of Ca²⁺ from CaCO₃, which is not soluble in water at neutral pH. Hence, by lowering the pH, the crosslinking reaction can be controlled by the liberated Ca²⁺. At lower temperatures, the crosslinking can be slower, resulting in hydrogels with well-ordered structure and enhanced gel strength. ALG with greater G content formed hydrogels with greater stiffness than those made with lower G content ALG.

Immobilization of Target

The hardened particle comprising a polymer framework functions to entrap or immobilize a target within the vessel. As used herein, the immobilization can refer to entrap the target so that it cannot diffuse out of the polymer framework. Immobilization of target can include physical incorporation of the target into the polymer framework, covalent tethering of the target to the polymer and/or monomer, or affinity binding of the target with the polymer framework.

Immobilization of Target: Physical Incorporation

When physically incorporating of a target into the polymer network, one of the design parameters to consider may be the mesh or pore size of the polymer networks that is modulated by the crosslink density. An increase in the crosslink density can decrease the average molecular distance between the adjacent crosslinks and thus can decrease hydrogel mesh size. Therefore, diffusivity of a target may decrease because smaller mesh size increases interactions of molecules with the polymer network and can act to confine molecules. On the other hand, hydrodynamic radius of a target can be increased to be larger than the pore size of the polymer network. For example, the target is bound to an immobilizable probe which is large in size. To make the immobilizable probe large in size, one can conjugate the immobilizable probe with long polymer tail, for example, an antibody immobilizable probe conjugated with one or more long PEG polymers. For another example, the immobilizable probe can further associate with a large particle which is incorporated into the polymer network.

Immobilization of Target: Covalent Tethering

Target (e.g., biological molecules) containing amine, carboxylic acid or thiol groups can be covalently bound to the functional groups of polymer framework precursors (e.g. polymers or monomers). This strategy can be used for sustaining their retention and/or release. For instance, the covalent incorporation of biological molecules such as proteins and nucleic acids permits their retention within hydrogel matrices, and their release can be controlled through hydrolytic and/or enzymatic cleavage of polymer-biological molecule bonds and/or networks. In some cases, a target is linked to the polymer network through an immobilizable probe, and in such cases, the immobilizable probe can be covalently bound to the network.

For example, a secondary amine of a target or an immobilizable probe can be reacted with carboxylic acid groups of a polyphosphazene polymer to form stable amide bonds. Lower pH (e.g., from 4.0 to 4.5, from 4.5 to 5.0, from 5.0 to 5.5, from 5.5 to 6.0, or from 6.0 to 6.5) can lead to degradation of the amide linkages. For another example, a target or an immobilizable probe bearing a hydroxyl group can be conjugated to carboxylic acid groups of polyphosphazene polymers, resulting in hydrolytic ester bonds. The hydrolytic degradation of the ester linkages can be controlled by pH.

Aldehyde groups can react with amine groups via Schiff's base reaction to form imine linkages that are degradable via hydrolysis, especially at low pH. For instance, an immobilizable probe can be conjugated to PEG-aldehyde to create a conjugate that can be subsequently bound to polymer networks composed of poly(vinyl amine) and PEG through Schiff's base reaction. The rapid hydrolysis of Schiff's base linkages at acidic pH can be used for controlled release in acidic environments.

Proteins containing primary amine groups can also be covalently incorporated into hydrogel networks, and the release of the proteins can be mediated via hydrolysis, reduction reaction and/or enzymatic degradation.

Immobilization of Target: Affinity Binding

Affinity is another way of associating targets within hydrogel networks which capitalizes on the association of one molecule to another via their opposite charges, hydrophobic interactions, hydrogen bonding, and/or van der Waals forces. Polymer networks functionalized or incorporated with molecules that have affinity with target molecules can serve to retain specific targets in hydrogels through these interactions.

Hydrophobic blocks of amphiphilic copolymers can interact with hydrophobic immobilizable probes via hydrophobic interactions in aqueous solutions, and therefore retain them within the polymer networks.

Ionic interactions naturally occurring between two oppositely charged molecules may be used for retaining target molecules or immobilizable probes within the polymer networks. For example, cationic polymers consisting of a high density of positively charged groups (such as PEI, polylysine, PAE and PAA) can condense negatively charged molecules into stabilized complexes. Exploiting this ionic binding phenomenon, functionalization of hydrogels with ionic molecules that can complex with counter-ionic biomolecules permits their retention within the polymer networks.

Aptamers, special types of single-stranded DNA or RNA oligonucleotides, can be used to recognize any targets of interest with high binding affinity and specificity. The affinity of aptamers to other molecules may provide a strategy to achieve desired retention or release of biomolecules. For example, aptamers can be conjugated to AAm gels to entrap the biomolecules that can bind by the aptamers.

In addition to aptamers, other biomolecules, such as peptides, that have affinity to target biological molecules can be incorporated into hydrogels to entrap target biological molecules. For example, PCLA-PEG-PCLA hydrogels can be functionalized with a peptide that can be bound by a protein target.

Functionalization of Polymer Framework

The functionalization of polymerizable or gellable polymers or monomers can be used to allow the specific capture of a given target. In certain cases, the desired functionality can be directly added in the prepolymer mixture and allowed to co-polymerize. A small amount of co-monomers with carboxyl or amine functionalities can be used during the polymerization step to allow functionalization of hydrogels in a post synthesis phase. This approach may be followed for conjugation of molecules that cannot withstand polymerization reaction conditions.

Examples of derivatization include carboxyl or amine groups, but other functionalities can also be used. Epoxy linkers can be introduced during the polymerization by using a suitable monomer while aldehydes or thiols groups can be introduced after post polymerization procedure in aqueous conditions. Click chemistry can be exploited for the functionalization. The polymer or monomer can be functionalized with a reactive group.

Reverse of Immobilization

In various embodiments, after barcoding a polynucleotide strand within a hardened particle, the barcoded polynucleotide can be released from the hardened particle. The releasing strategies can be any strategy described herein to reverse the interaction between the immobilizable probe or the target and the polymer network. In some situations, the barcoded polynucleotide is the target which is associated with the polymer network through the immobilizable probe, the releasing of the barcoded polynucleotide can be achieved by reversing the interaction between the immobilizable probe and the polymer network. Reversing the interaction between the immobilizable probe and the polymer network can be achieved through an external stimuli based on the type of the polymer framework. For example, the external stimuli can be pH, temperature, a chemical, or a light signal. In some situations, the barcoded polynucleotide is a target polynucleotide which hybridizes with the immobilizable probe, the barcoded polynucleotide can be released by simply heating the hardened particle. In some situations, the barcoded polynucleotide is a polynucleotide covalently linked to a probe through a reactive group, and the barcoded polynucleotide can be released by reversing the covalent bond.

In some embodiments, a covalent bond linking the barcoded polynucleotide to the polymer framework of the hydrogel can be broken. For example, the immobilizable probe may comprise a disulfide bond or a photo-cleavable bond that stably links the barcoded polynucleotide to the polymer framework. In these situations, the disulfide bond can be cleaved by reducing reagent such as DTT, and the photo-cleavable bond can be cleaved by illumination with light of appropriate wavelength and intensity.

In some embodiments, one or a series of non-covalent bond linking the barcoded polynucleotide to the polymer framework of the hydrogel can be broken. For example, stable nucleic acid hybridization (a form of non-covalent interaction) may be essential to stably link the compartment-barcoded polynucleotide to the polymer framework. Such nucleic acid hybridization, which may be stable at room temperature, can be broken by higher temperature (e.g., at least 70° C., 75° C., 80° C., 85° C., 90° C., 95° C., or higher). Thus, heating can break the linkage between the compartment-barcoded polynucleotide and the polymer framework. The barcoded polynucleotide that is no longer stably linked to the polymer framework is called “released barcoded polynucleotide.” In some embodiments, the released compartment-barcoded polynucleotide can exit the hydrogel beads into the surrounding solution via diffusion. In some embodiments, electrophoretic field may be applied to facilitate the exit of the released compartment-barcoded polynucleotide. In some embodiments, the polymer framework can be broken to melt the hydrogel bead. For example, disulfide bonds may be used to maintain the gel state of the hydrogel beads. In this situation, reducing agents, such as DTT, dithiobutylamine, and Tris(2-carboxyethyl)phosphine, can be used to break the disulfide bonds thus melt the hydrogel. For another example, peptides may crosslink in the polymer framework essential to maintain the gel state of the hydrogel beads. In this situation, proteases or peptidases that can cleave the peptide may be used to melt the hydrogel bead. It should be noted that when the hydrogel is melted, the barcoded polynucleotide can be freely accessible by other reagents in solution even if the linkage between the barcoded polynucleotide and the polymer framework is not cut. For another example, increasing the temperature of hydrogel particles can be used to melt thermo-sensitive hydrogel particles (e.g., agarose gels). In some cases, the temperature used to melt thermo-sensitive hydrogel particles is at least 50° C., 55° C., 60° C., 65° C., 70° C., 75° C., 80° C., 85° C., 90° C., 95° C., 100° C., or higher.

Barcode-Accepting Polynucleotide

The compositions and methods provided herein comprise a vessel comprising a barcode-accepting polynucleotide. The barcode-accepting polynucleotide is a nucleotide sequence located on either 5′ end or 3′ end that can be attached with a barcode sequence. Examples of barcode-accepting polynucleotide are illustrated in FIGS. 4C-D and FIGS. 6A-D. The barcode-accepting polynucleotide can be the target, a complementary strand of the target polynucleotide, a nucleotide sequence of the immobilizable probe, or a nucleotide sequence of a detection probe that can bind to an immobilized target. In some cases, the barcode-accepting polynucleotide is a non-cellular polynucleotide.

The barcode-accepting polynucleotide can denote the identity of the target. In some embodiments, the barcode-accepting polynucleotide is an mRNA molecule, a miRNA molecule, a non-coding RNA molecule, or a circular RNA molecule. Since in many applications one is interested in the quantity or sequence of such RNA molecules, these RNA molecules are considered target molecules. In some embodiments, the barcode-accepting polynucleotide is a polynucleotide that is copied or amplified from the target. For example, it may be a cDNA molecule copied from an mRNA molecule wherein the mRNA molecule is the target. In some embodiments, the barcode-accepting polynucleotide is an artificially synthesized polynucleotide comprising a sequence that is designed to reflect the identity of a polynucleotide target (e.g., miRNA) or a non-polynucleotide target (e.g., protein). In the example shown in FIG. 6A, one may synthesize a 20 nt- to 100 nt-long polynucleotide (604) with an arbitrary sequence designated to reflect the identity of the target protein (602) (i.e., the target), and conjugate this polynucleotide to an antibody (603) that can stably bind the target. This polynucleotide can serve as the barcode-accepting polynucleotide.

The barcode-accepting polynucleotide can be extended (via primer extension or ligation, at its 5′ end or 3′ end) to contain a barcode sequence to form a continuous, amplifiable polynucleotide that comprise both (a) the sequence that denotes the identity of the target and (b) the barcode. For example, in the case shown in FIG. 4B, the sequence to the left of domain “3A-a” contains sequence originated from a target RNA molecule thus denoting the target, and BC-a and BC-b are barcode sequences. The bottom strand of the bottom molecule in FIG. 4B (i.e., product of Step S4.5) is a continuous, amplifiable polynucleotide.

Barcode

A barcode or barcode sequence relates to a natural or synthetic nucleic acid sequence comprised by a polynucleotide allowing for unambiguous identification of the polynucleotide and other sequences comprised by the polynucleotide having the same barcode sequence. The barcode can be any suitable length, such as 2 to 100 nucleotides in length. The barcode can have random sequences or pre-determined sequences. A barcode sequence can comprise a sequence of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 45, at least 50, at least 55, at least 60, at least 65, at least 70, at least 75, at least 80, at least 85, at least 90, at least 95, or at least 100 consecutive nucleotides. A barcode sequence can comprise a randomly assembled sequence of nucleotides. A barcode sequence can be a degenerate sequence. A barcode sequence can be a known sequence. A barcode sequence can be a predefined sequence. A barcode can comprise one or more barcode segments, wherein the one or more barcode segments are consecutive or separated by one or more predefined sequences.

In some embodiments, a barcode is a vessel barcode. A vessel barcode can comprise information that is unique to polynucleotides from a single cell or from a single vessel, compared to polynucleotides from a different single cell or from a different single vessel. Therefore, a vessel barcode can be a single cell barcode or a barcode for a single complex of cells. In some embodiments, a vessel barcode is a single organelle barcode.

A barcode can have a length within a range of from 2 to 36 nucleotides, or from 4 to 36 nucleotides, or from 6 to 30 nucleotides, or from 8 to 20 nucleotides, or from 2 to 20 nucleotides, or from 4 to 20 nucleotides, or from 6 to 20 nucleotides. In some embodiments, a barcode can have a length within a range of from 3 to 10 nucleotides, or from 10 to 50 nucleotides, or from 50 to 100 nucleotides. In certain aspects, the melting temperatures of barcodes within a set are within 10° C. of one another, within 5° C. of one another, or within 2° C. of one another. In certain aspects, the melting temperatures of barcodes within a set are not within 10° C. of one another, within 5° C. of one another, or within 2° C. of one another. In other aspects, barcodes are members of a minimally cross-hybridizing set. For example, the nucleotide sequence of each member of such a set can be sufficiently different from that of every other member of the set that no member can form a stable duplex with the complement of any other member under stringent hybridization conditions. In some embodiments, the nucleotide sequence of each member of a minimally cross-hybridizing set differs from those of every other member by at least two nucleotides. Barcode technologies are described in Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240.

In some cases, a barcode sequence or segment can be flanked by a predefined sequence on 5′ and/or 3′ side of the barcode sequence or segment. For example, a barcode sequence can be flanked by a barcode acceptor site and a barcode donor site in order to attach the barcode sequence on to a polynucleotide strand. A barcode-containing segment can refer to the predefined sequence. In some cases, a barcode segment can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 45, or at least 50 nucleotides in length. In some cases, a barcode segment can be from 2 to 4, from 3 to 10 nucleotides, from 5 to 10, from 6 to 12, from 10 to 15, from 15 to 20, from 20 to 30, from 30 to 40, from 40 to 50, or from 10 to 50 nucleotides in length. The predefined sequence flanking a barcode segment can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 45, or at least 50 nucleotides in length. In some cases, the predefined sequence flanking a barcode segment can be can be from 2 to 4, from 3 to 10 nucleotides, from 5 to 10, from 6 to 12, from 10 to 15, from 15 to 20, from 20 to 30, from 30 to 40, from 40 to 50, or from 10 to 50 nucleotides in length. A barcode-containing segment (barcode plus flanking sequences) can be at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 45, at least 50, at least 55, at least 60, at least 65, at least 70, at least 75, at least 80, at least 85, at least 90, at least 95, or at least 100 consecutive nucleotides.

A vessel barcode can comprise any length of nucleotides. For example a vessel barcode can comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or at least about 1000 nucleotides. For example a vessel barcode can comprise at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides. In some embodiments, a vessel barcode has a particular length of nucleotides. For example, a vessel barcode can be about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length.

In some embodiments, each vessel barcode in a plurality of vessel barcodes has at least about 2 nucleotides. For example, each vessel barcode in a plurality of vessel barcodes can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length. In some embodiments, each vessel barcode in a plurality of vessel barcodes has at most about 1000 nucleotides. For example, each a vessel barcode in a plurality of vessel barcodes can be at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length. In some embodiments, each a vessel barcode in a plurality of vessel barcodes has the same length of nucleotides. For example, each a vessel barcode in a plurality vessel barcodes can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length. In some embodiments, one or more vessel barcodes in a plurality of vessel barcodes have a different length of nucleotides. For example one or more first vessel barcodes in a plurality of vessel barcodes can have about, or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides and one or more second vessel barcodes in a plurality of vessel barcodes can have about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides, wherein the number of nucleotides of the one or more first vessel barcodes is different than the one or more second vessel barcodes.

The number of different vessel barcodes can be less than the total number of molecules to be labeled with barcodes in a plurality of vessels. In some embodiments, the number of different vessel barcodes is at least about 1, 1.5, 2, 2.5, 3, 3.5, 4, 4.5, 5, 6, 7, 8, 9, 10, 15, 20, 30, 40, 50, 60, 70, 80, 90, or 100 times less than the total number of molecules to be labeled in a plurality of vessels.

The number of amplified product molecules from a vessel barcoded polynucleotide molecule in a single vessel can be in excess of the number of different molecules to be labeled in the single vessel. In some embodiments, the number of amplified product molecules from a vessel barcoded polynucleotide molecule in a single vessel is at least about 1, 1.5, 2, 2.5, 3, 3.5, 4, 4.5, 5, 6, 7, 8, 9, 10, 15, 20, 30, 40, 50, 60, 70, 80, 90, or 100 times greater than the number of different molecules to be labeled in the single vessel.

The number of vessel barcoded polynucleotide molecules in a single vessel can be less than the number of different molecules to be labeled in the single vessel. In some embodiments, the number of vessel barcoded polynucleotide molecules in a single vessel is at least about 1, 1.5, 2, 2.5, 3, 3.5, 4, 4.5, 5, 6, 7, 8, 9, 10, 15, 20, 30, 40, 50, 60, 70, 80, 90, or 100 times less than the number of different molecules to be labeled in the single vessel.

The number of vessel barcoded polynucleotide molecules in a single vessel can be one molecule. The number of unamplified vessel barcoded polynucleotide molecules in a single vessel can be one molecule.

The vessel barcodes in a population of vessel barcodes can have at least 10, 15, 20, 25, 30, 35, 40, 45, 50, 60, 70, 80, 90, 100, 200, 300, 400, 500, 600, 700, 800, 900, 1000 or more different sequences. For example, the vessel barcodes in a population can have at least 2,000, 3,000, 4,000, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1,000,000 or more different sequences. Thus, a plurality of vessel barcodes can be used to generate at least 10, 15, 20, 25, 30, 35, 40, 45, 50, 60, 70, 80, 90, 100, 200, 300, 400, 500, 600, 700, 800, 900, 1000 or more different sequences from one or more polynucleotides, such as target polynucleotides. For example, a plurality of vessel barcodes can be used to generate at least 2,000, 3,000, 4,000, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰10, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² or more different sequences from one or more polynucleotides, such as target polynucleotides. For example, a plurality of vessel barcodes can be used to generate at least about 10, 15, 20, 25, 30, 35, 40, 45, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10^{8 8}, 3×10, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10^{9 9}, 7×10, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10^{12 12}, 4×10, 5×10¹², 6×10¹², 7×10¹², 8×10¹², 9×10¹² or more different sequences from at least about 10, 15, 20, 25, 30, 35, 40, 45, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10^{7 7}, 9×10, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10^{9 9}, 4×10, 5×10⁹, 6×10⁹, 7×10⁹, 8×10⁹, 9×10⁹, 1×10¹⁹, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² or more target polynucleotides.

In some embodiments, one or more vessel barcodes are used to group or bin sequences. In some embodiments, one or more vessel barcodes are used to group or bin sequences, wherein the sequences in each bin contain the same vessel barcode. In some embodiments, one or more vessel barcodes are used to group or bin sequences, wherein the sequences in each bin comprise one or more amplicon sets. In some embodiments, one or more vessel barcodes are used to group or bin sequences, wherein the sequences in each bin comprise a plurality of sequences wherein the polynucleotides from which the plurality of sequences are generated are derived from the polynucleotides from a single vessel or single cell. The hardened particles provided herein may further comprise unique molecular identifier (UMI) which functions to denote a single molecule. The hardened particle may comprise species barcode which functions to denote a group of molecules, for example, a group of phosphorylated proteins.

The barcode can be linked to the target and/or the immobilizable probe. The barcode can be linked to a barcode-accepting polynucleotide. In some cases, the barcode-accepting polynucleotide is the target polynucleotide, and then the barcode is directly attached to the 5′ or 3′ end of the target polynucleotide. In some cases, the barcode-accepting polynucleotide is a complementary strand of the target polynucleotide, and then the barcode is directly attached to the 5′ or 3′ of the complementary strand of the target polynucleotide. In some cases, the barcode-accepting polynucleotide is the immobilizable probe or is linked to the immobilizable probe. In some cases, the barcode-accepting polynucleotide is attached to a detection probe (e.g., FIGS. 6A and 6D).

Barcoding

In the present disclosure, after polymerizing or gelling the vessel comprising one or more immobilized targets, a vessel barcode is added to each hardened particle. The barcode can be attached to a polynucleotide strand. In some cases, the polynucleotide strand attached with the barcode is the barcode-accepting polynucleotide. In some cases, the barcode is synthesized onto the barcode-accepting polynucleotide. In some cases, the barcode is ligated to the barcode-accepting polynucleotide. In a composition comprising a plurality of hardened particles, after barcoding, each single hardened particle will comprise one or more copies of a unique barcode sequence to denote one or more targets from that single vessel. In this composition, each two hardened particles will have different barcode sequences.

In some embodiments, the barcoding strategy can involve co-encapsulation of hardened particle comprising a target along with beads coated with barcoded primers into droplets.

In some embodiments, the barcoding strategy can involve co-encapsulation of hardened particle comprising a target along with a hydrogel particle carrying copies of a unique barcode that can be released from the hydrogel particle. For example, for single-cell transcriptomic analysis, the hardened particle comprising mRNA targets can be co-encapsulated with a reverse transcription (RT) mix and hydrogel particles carrying primers that can be released upon UV excitation.

In some embodiments, the barcoding strategy can involve combinatorial split-and-pool synthesis. For example, the hardened particles are pooled and then are repeatedly split into four oligonucleotide synthesis reactions, to which one of the four DNA bases is added, and then pooled together after each cycle. In this strategy, a total of “N” split-pool cycles can result in 4^{N} possible sequences. Similarly, the hardened particle can also be pooled and split into different reactions, each of the reaction containing a unique barcode segment sequence of a variety of barcode sequences. After the first cycle, each hardened particle will comprise a unique first barcode segment, and the hardened particles are pooled and split again for adding a second barcode segment. In this strategy, if a total of “M” varieties of reactions are performed in each cycle and a total of “N” split-pool cycles are performed, it can result in M^{N} possible sequences.

The barcoding methods provided herein can comprise: pooling the plurality of hardened particle; dividing the plurality of hardened particle into separate first populations; attaching a first oligonucleotide comprising a first barcode sequence segment to the separate first populations, wherein each separate first population comprises a different first barcode sequence segment; pooling the separate first populations to provide a first pooled population; separating the first pooled population into a plurality of second populations; attaching a second oligonucleotide comprising a second barcode sequence segment to the first oligonucleotide already attached to the second populations, wherein each of the plurality of second populations comprises a different second barcode sequence segment; and pooling the plurality of second populations to provide a second pooled population comprising a barcode library.

In some cases, a method of barcoding can comprise: generating a population of vessels comprising a first total number of a subpopulation of vessels, each of the first subpopulation comprising (i) a target from a single cell, a single complex of cells, or a single exosome; and (ii) a plurality of polymerizable or gellable polymers and/or monomers; polymerizing or gelling the plurality of polymerizable or gellable polymers and/or monomers of the population of vessels, thereby forming a population of hardened particles; and contacting a barcode to the population of hardened particles, thereby forming a population of barcoded hardened particles comprising a second total number of a subpopulation of barcoded hardened particles, each of the subpopulation comprising (i) the target from the single cell, the single complex of cells, or the single exosome; and (ii) polymerized or gelled polymers/monomers; wherein the second total number is at least 90%, 91%, 92%, 93%, 94%, 95%, 96%, 97%, 98%, 99%, or at least 100% of the first total number. In some embodiments, the second total number is from 80% to 85%, from 85% to 90%, from 90% to 95%, from 95% to 98%, or from 98% to 100%, of the first total number,

In some cases, a method provided herein comprises: generating a population of vessels, each comprising a plurality of polymerizable or gellable polymers or monomers, wherein a subpopulation of the population comprises (i) a target from a single cell, a single complex of cells or a single exosome, and (ii) a non-cellular polynucleotide; and polymerizing or gelling the plurality of polymerizable or gellable polymers or monomers of the population, wherein each vessel of the subpopulation comprises a hardened particle. In some cases, a barcode is contacted with the hardened particle.

Compositions

The composition provided herein can comprise a plurality of vessels, each vessel of the plurality comprising a hydrogel particle or bead; an immobilizable probe embedded within the hydrogel particle or the bead, wherein the immobilizable probe is immobilized to a diffusion restricting agent; and a target embedded within the hydrogel particle or the bead and bound to the immobilizable probe, wherein the target is from a single cell, a single complex of cells, or a single exosome; wherein the hydrogel or bead has a volume that is at least 50% of a volume of the vessel. The hydrogel or bead can have a volume that is at least 50%, at least 60%, at least 65%, at least 70%, at least 75%, at least 80%, at least 85%, at least 90%, at least 95%, at least 98% of the volume of the vessel. In some cases, the composition can further comprise a lysing agent.

The composition provided herein can comprise a plurality of hydrogel particles or beads, each hydrogel particle or bead of the plurality comprising an immobilizable probe embedded within the hydrogel particle or bead, wherein the immobilizable probe is immobilized to a diffusion restricting agent or has a hydrodynamic radius that is larger than a pore size of the hydrogel particle or bead; a target embedded within the hydrogel particle or bead and bound to the immobilizable probe, wherein the target is from a different single cell, a different single complex of cells, or a different single exosome; and a barcode.

A vessel or hardened particle can comprise a polymerized or gelled plurality of polymers and/or monomers; a single cell, a single complex of cells, or a single exosome; an immobilizable probe linked to one or more polymers and/or monomers of the polymerized or gelled plurality; and a barcode.

A vessel or hardened particle can comprise: a polymerized or gelled plurality of polymers and/or monomers; a single cell, a single complex of cells, or a single exosome; an immobilizable probe, wherein the immobilizable probe has a hydrodynamic radius that is larger than a pore size of the vessel; and a barcode.

A droplet can comprise: a single cell, a single complex of cells, or a single exosome; a plurality of polymerizable or gellable polymers and/or monomers; an immobilizable probe linked to one or more polymers and/or monomers of the plurality; and a non-cellular polynucleotide; wherein the non-cellular polynucleotide is linked to the immobilizable probe.

The hydrogel particle or bead can comprise a polymerized or gelled plurality of polymers and/or monomers. The immobilizable probe can comprise an immobilization moiety. The immobilization moiety can link the immobilizable probe to one or more polymers and/or monomers of the polymerized or gelled plurality. The immobilization moiety can be a chemical that is co-polymerized with the polymers and/or monomers, a chemical or protein that stably interacts with the polymers and/or monomers directly, or a chemical or protein that stably interacts with the polymers and/or monomers indirectly. Examples of the immobilization moiety include, but are not limited to, an NHS ester, a biotin, a maleimide group, a thiol group, an azide group, an avidin or streptavidin, a single-stranded polynucleotide, a biotin group, can a methacryl group. The diffusion restricting agent can be one or more polymers and/or monomers of the polymerized or gelled plurality. The diffusion restricting agent can be a particle larger than a pore size of the hydrogel or bead. The diffusion restricting agent can be a polymer chain conjugated on the immobilizable probe. The target can be a target polynucleotide, a target polypeptide, a target organelle, or any combination thereof. The target can be a target molecule. The target polynucleotide can be a target deoxyribonucleic acid (DNA), a target ribonucleic acid (RNA), or a combination thereof. In some cases, the target DNA is a genomic DNA, a modified DNA, or a combination thereof. In some cases, the modified DNA is a methylated DNA. In some cases, the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. The composition can further comprise a complementary strand of the target polynucleotide. The complementary strand can be a cDNA strand. The target organelle can be an acrosome, an autophagosome, a centriole, a cilium, a cnidocyst, an eyespot apparatus, a glycosome, a glyoxysome, a hydrogenosome, a lysosome, a melanosome, a mitosome, a myofibril, a nucleolus, a parenthesome, a peroxisome, a proteasome, a ribosome (80S), a stress granule, a vesicle, a chloroplast, an endoplasmic reticulum, a flagellum, a Golgi apparatus, a mitochondrion, a nucleus, a vacuole, or any combination thereof. The target polypeptide can be a peptide, a protein or a portion thereof, a modified protein or a portion thereof, a protein complex, or any combination thereof.

The immobilizable probe can further comprise a targeting moiety. The targeting moiety can be a polynucleotide, a polypeptide, or a chemical group. The polynucleotide can be a primer or an oligonucleotide aptamer. In some cases, the primer is a reverse transcription primer. In some cases, the reverse transcription primer comprises a poly-deoxy-thymidine nucleotide sequence. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. In some cases, the reactive group is an N-hydroxysuccinimide (NHS) ester, a maleimide group, a hydrazide group, an azide group or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine. In some cases, the lysing agent is a cell lysing agent. In some cases, the different single cell, the different single complex of cells, or the different single exosome is lysed.

Methods

According to an aspect, a method of barcoding can comprise: contacting a barcode to a hardened particle comprising a polymerized or gelled polymers and/or monomers and a single cell, a single complex of cell, a single exosome, a target, or a combination thereof, wherein the barcode diffuses into the hardened particle. The method can further comprise forming a compartment. In some cases, the compartment can be formed before contacting the barcode. The compartment can comprise a plurality of polymerizable or gellable polymers and/or monomers; the single cell, the single complex of cells, the single exosome, the target, or a combination thereof and an immobilizable probe. In some cases, the method further comprises polymerizing or gelling the plurality of polymers and/or monomers in the compartment, thereby forming the hardened particle. The compartment or the hardened particle can further comprise a crosslinking agent. The compartment or the hardened particle can further comprise a polymerization initiator. The polymerization initiator can be a photo-initiator. In some cases, the polymerization initiator is ammonium persulfate (APS), N,N,N′,N′-tetramethylethane-1,2-diamine (TEMED), Lithium- and magnesium phenyl-2,4,6-trimethylbenzoylphosphinates (TMPPL and TMPPM), sodium 4-[2-(4-morpholino)benzoyl-2-dimethylamino]-butylbenzenesulfonate (MBS), methylated-O-cyclodextrin (MβCD), or 2,2-dimethoxy-2-phenyl acetophenone (DMPA), or any combination thereof. In some cases, a thin liquid layer surrounding the hardened particle is removed. In some cases, the single cell, the single complex of cells, or the single exosome is lysed. In some cases, the target is from the single cell, the single complex of cells, or the single exosome. In some cases, the target is bound to the immobilizable probe. In some cases, the target is a target polynucleotide, a target polypeptide, a target organelle, or any combination thereof. In some cases, the target polynucleotide is a target deoxyribonucleic acid (DNA), a target ribonucleic acid (RNA), or a combination thereof. In some cases, the target DNA is a genomic DNA, a modified DNA, or a combination thereof. In some cases, the modified DNA is a methylated DNA. In some cases, the target RNA is target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. In some cases, the method further comprises a complementary strand of the target polynucleotide. In some cases, the complementary strand is a cDNA strand. In some cases, the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some cases, the target polypeptide is a peptide, a protein or a portion thereof, a modified protein or a portion thereof, a protein complex, or any combination thereof. In some cases, the immobilizable probe comprises a targeting moiety. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotide sequence. In some cases, the method further comprises annealing the primer to the target polynucleotide. In some cases, the method further comprises extending the primer to generate a complementary strand of the target polynucleotide. In some cases, the extending is performed before or after the polymerizing or gelling of the polymers and/or monomers. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. In some cases, the reactive group is an N-hydroxysuccinimide (NHS) ester, a maleimide group, a hydrazide group, an azide group, or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine. In some cases, the immobilizable probe comprises an immobilization moiety. In some cases, the immobilization moiety links the immobilizable probe to one or more of the polymers and/or monomers of the polymerized or gelled plurality. In some cases, the immobilization moiety wherein the immobilization moiety is a chemical that is co-polymerized with the polymers and/or monomers, a chemical or protein that stably interact with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the barcode is attached to the immobilizable probe, the target, or the complementary strand of the target polynucleotide. In some cases, the compartment or the hardened particle further comprises a barcode-accepting polynucleotide. In some cases, the barcode is attached to the barcode-accepting polynucleotide. In some cases, barcode-accepting polynucleotide is the target, the complementary strand of the target, or the immobilizable probe. In some cases, the compartment or the hardened particle further comprises a detection probe. In some cases, the barcode is attached to the detection probe. In some cases, the barcode is from 5 to 500 nucleotides in length. In some cases, the hardened particle is a plurality of hardened particle. In some cases, adding the barcode to the plurality of hardened particle comprises: pooling the plurality of hardened particle; dividing the plurality of hardened particle into separate first populations; attaching a first oligonucleotide comprising a first barcode sequence segment to the separate first bead populations, wherein each separate first population comprises a different first barcode sequence segment; pooling the separate first populations to provide a first pooled population; separating the first pooled population into a plurality of second populations; attaching a second oligonucleotide comprising a second barcode sequence segment to the first oligonucleotide already attached to the second populations, wherein each of the plurality of second populations comprises a different second barcode sequence segment; and pooling the plurality of second populations to provide a second pooled population comprising a barcode library. In some cases, the first barcode sequence segment or the second barcode sequence segment comprises at least 1 nucleotide in length. In some cases, the first barcode sequence segment or the second barcode sequence segment comprises at least 4 nucleotides in length. In some cases, the first barcode sequence segment or the second barcode sequence segment comprises from about 4 to about 20 nucleotides in length. In some cases, the method further comprises washing the hardened particles extensively.

According to another aspect, a method of barcoding can comprise: (a) generating a population of vessels comprising a first total number of a subpopulation of vessels, each of the first subpopulation comprising (i) a target from a single cell, a single complex of cells, or a single exosome; and (ii) a plurality of polymerizable or gellable polymers and/or monomers; (b) polymerizing or gelling the plurality of polymerizable or gellable polymers and/or monomers of the population of vessels, thereby forming a population of hardened particles; and (c) contacting a barcode to the population of hardened particles, thereby forming a population of barcoded hardened particles comprising a second total number of a subpopulation of barcoded hardened particles, each of the subpopulation comprising (i) the target from the single cell, the single complex of cells, or the single exosome; and (ii) polymerized or gelled polymers/monomers; wherein the second total number is at least 90%, 91%, 92%, 93%, 94%, 95%, 96%, 97%, 98%, 99%, or at least 100% of the first total number.

According to another aspect, a method provided herein can comprise: generating a population of vessels, each comprising a plurality of polymerizable or gellable polymers or monomers, wherein a subpopulation of the population comprises (i) a target from a single cell, a single complex of cells or a single exosome, and (ii) a non-cellular polynucleotide; and polymerizing or gelling the plurality of polymerizable or gellable polymers or monomers of the population, wherein each vessel of the subpopulation comprises a hardened particle.

According to another aspect, a method provided herein can comprise: (a) generating a population of vessels, each comprising a plurality of polymerizable or gellable polymers or monomers, wherein a subpopulation of the population comprises a single cell, a single complex of cells or a single exosome; (b) polymerizing or gelling the plurality of polymerizable or gellable polymers or monomers of the population, wherein each vessel of the subpopulation comprises a hardened particle; and (c) lysing the single cell, the single complex of cell, or the single exosome.

A method of barcoding a target can comprise: (a) forming a plurality of vessels each comprising: a plurality of polymerizable or gellable polymers and/or monomers; a target; (b) polymerizing or gelling the polymers and/or monomers in each of the plurality of vessels to form a hardened particle, thereby forming a plurality of hardened particles; (c) generating a barcode sequence in each of the plurality of hardened particles, thereby generating a barcoded target sequence. Each of the plurality of vessels can further comprise a single cell, a single complex of cells, or a single exosome. The target can be from the single cell, the single complex of cells, or the single exosome. The target can be a target polynucleotide, a target polypeptide, or a target organelle. The target polynucleotide can be a target ribonucleic acid (RNA) or a target deoxyribonucleic acid (DNA). The target RNA can be total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof. The target DNA can be a genomic DNA, a modified DNA, or a combination thereof. In some cases, the modified DNA is a methylated DNA. The target organelle can comprise acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof. In some cases, the target polypeptide is a peptide, a protein or a portion thereof, a modified protein or a portion thereof, a protein complex, or any combination thereof. In some cases, each of the plurality of vessels further comprises an immobilizable probe linked to one or more polymerizable or gellable polymers or monomers of the plurality. In some cases, the immobilizable probe comprises a targeting moiety. In some cases, the targeting moiety is a polynucleotide, a polypeptide, or a chemical group. In some cases, the polynucleotide is a primer or an oligonucleotide aptamer. In some cases, the primer is a reverse transcription primer. In some cases, the reverse transcription primer comprises a poly-deoxy-thymidine nucleotide sequence. In some cases, the method further comprises annealing the primer to the target polynucleotide. In some cases, the method further comprises extending the primer to generate a complementary strand of the target polynucleotide. In some cases, the extending is performed in the vessel or the hardened particle. In some cases, the polypeptide is an antibody or a fragment thereof, or a peptide aptamer. In some cases, the chemical group is a reactive group. In some cases, the reactive group forms a covalent bond with an amino acid side chain or a nucleobase of the target. In some cases, the reactive group is an N-hydroxysuccinimide (NHS) ester, a maleimide group, a hydrazide group, an azide group, or Label-IT linker and reactive group. In some cases, the amino acid side chain is lysine or cysteine side chain. In some cases, the amino acid side chain comprises an amine group, a carboxylate group, a carbonyl group, or a sulfhydryl group. In some cases, the nucleobase is guanine. In some cases, the immobilizable probe comprises an immobilization moiety. In some cases, the immobilization moiety links the immobilizable probe to one or more of the polymers and/or monomers of the plurality. In some cases, the immobilization moiety a chemical that is co-polymerized with the polymers and/or monomers, a chemical or protein that stably interact with the polymers and/or monomers directly, or a chemical or protein that stably interacts with the polymers and/or monomers indirectly. In some cases, the immobilization moiety is an NHS ester, a biotin, a maleimide group, a thiol group, an azide group, an avidin or streptavidin, a single-stranded polynucleotide, a biotin group, or a methacryl group. In some cases, the method further comprises contacting the plurality of vessels or hardened particles to a lysing agent. In some cases, the lysing agent comprises IGEPAL CA-630, Sarkosyl, protease K, lyticase, glusulase and zymolyase, or any combination thereof. In some cases, the barcode sequence diffuses into the plurality of hardened particles. In some cases, the vessel or the hardened particle further comprises a barcode-accepting polynucleotide. In some cases, the barcode is attached to the barcode-accepting polynucleotide. In some cases, the barcode-accepting polynucleotide is the target, the complementary strand of the target, or the immobilizable probe. In some cases, the vessel or the hardened particle further comprises a detection probe. In some cases, the barcode-accepting probe is attached to the detection probe. In some cases, generating the barcode sequence comprises: pooling the plurality of hardened particle; dividing the plurality of hardened particle into separate first populations; attaching a first oligonucleotide comprising a first barcode sequence segment to the separate first populations, wherein each separate first population comprises a different first barcode sequence segment; pooling the separate first populations to provide a first pooled population; separating the first pooled population into a plurality of second populations; attaching a second oligonucleotide comprising a second barcode sequence segment to the first oligonucleotide already attached to the second populations, wherein each of the plurality of second populations comprises a different second barcode sequence segment; and pooling the plurality of second populations to provide a second pooled population comprising a barcode library. In some cases, the first barcode sequence segment or the second barcode sequence segment comprises at least 1 nucleotide in length. In some cases, the first barcode sequence segment or the second barcode sequence segment comprises at least 4 nucleotides in length. In some cases, the first barcode sequence segment or the second barcode sequence segment comprises from about 4 to about 20 nucleotides in length. In some cases, the barcode sequence is attached to the immobilizable probe, the target, or the complementary strand of the target polynucleotide. In some cases, the method further comprises washing the hardened particles extensively. In some cases, the single complex of cells comprises a first cell and a second cell. In some cases, the first cell is a mammalian cell. In some cases, the mammalian cell expresses a T-cell receptor or a portion thereof. In some cases, the first cell is an immune cell. In some cases, the immune cell is a T cell. In some cases, the second cell is an antigen presenting cell. In some cases, the second cell is a yeast cell. In some cases, the yeast cell expresses a MHC molecule on its surface. In some cases, the MHC molecule is a class I MHC or a class II MHC. In some cases, the MHC molecule is expressed from a gene selected from HLA-A, HLA-B, HLA-C, HLA-DPA1, HLA-DPB1, HLA-DQA1, HLA-DQB1, HLA-DRA, HLA-DRB1, or any combination thereof. In some cases, the MHC molecule further comprises a peptide. In some cases, the method further comprises sequencing the barcoded target sequence. In some cases, the vessel is a water-in-oil droplet. In some cases, the hardened particle is a hydrogel particle.

The methods described herein can be used to detect secreted proteins from live cells with single-cell resolution. For example, using the methods (e.g., Example 4), more than 1,000, more than 10,000, more than 100,000 or more than 1,000,000 agarose hydrogel beads some of which comprise one live cell can be prepared. The agarose can have low gelling temperature so that the agarose remains melt at about 20° C., 25° C., 30° C., 32° C., 35° C., 37° C., or about 40° C. Some of the cell may secrete a protein of interest. The agarose gel can comprise culture media or serum to keep the cells alive. Capture antibodies, which can bind the secreted protein of interest stably, can be stably attached or entrapped in the agarose gel. The capture antibodies can be attached to the polysaccharide chain, or can be attached to a particulate or soluble diffusion restricting agent (e.g., a linear polymer). The attachment of the capture antibody can be mediated by oligonucleotides. For example, a ‘host’ oligonucleotide may be stably attached or entrapped in the agarose gel (as in Example 4), and a ‘guest’ oligonucleotide may be attached to the antibody, where the host and guest oligonucleotides can stably hybridize. Multiple capture antibodies (e.g., more than 2, more than 3, more than 4, more than 5, more than 10, more than 15, more than 20) can be used to capture multiple secreted proteins of interest.

Detection antibodies, which can bind the secreted protein of interest bound to the capture antibody (i.e., in a sandwich format), can be used to treat the agarose beads. The detection antibodies can diffuse into the agarose gel beads and bind the proteins of interest immobilized by the capture antibodies. The detection antibody may be labeled with a barcode (e.g., FIG. 6A). Alternatively, the detection antibody may be attached with a detectable moiety such as a fluorophore, a biotin moiety, or a signal-amplifying enzyme such as horseradish peroxidase (HRP) and alkaline phosphatase (AP), allowing detection methods such as flow cytometry or microscopy. In the case of flow cytometry, agarose gel beads housing cells expressing desirable level(s) of one or more proteins of interest can be sorted using a FACS.

### DETAILED DESCRIPTION OF FIGURES

FIG. 1 illustrates an example of vessel formation and hardened particle generation. Shown here is a vessel (e.g. a droplet, 101) comprising a plurality of polymerizable or gellable polymers (103) and/or monomers (102), an immobilizable probe (104), and a biological particle (e.g. a single cell, 105). The biological particle contains a target (e.g. nucleic acids, 106). The biological particle is further lysed (107). The plurality of polymerizable or gellable polymers are then polymerized or gelled to form a hardened particle (109) comprising a polymer framework (108). The immobilizable probe (104) captures the target (106) and immobilizes the target on the framework. The immobilizable probe shown here in this example is a primer. The primer is further extended. A barcode (110) is attached to the 3′ end of the extended primer (or the complementary strand of the target).

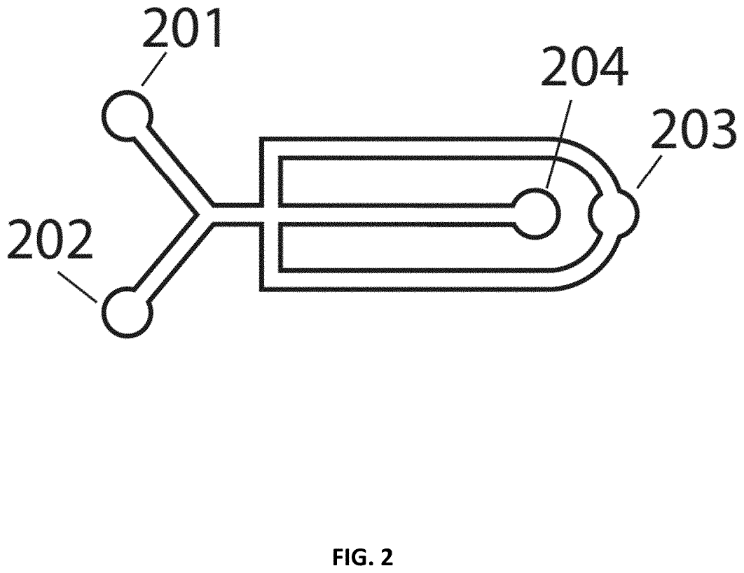

FIG. 2 illustrates an example of microfluidic device that can be used for vessel generation. For example, the cell suspension and the Cell Lysing and mRNA Embedding Buffer may be infused from incoming ports 201 and 202, respectively, at the same rate, causing 1:1 mixture of the fluids in the droplets. The carrier oil (such as RAN Biotechnologies, cat. no. 008-FluoroSurfactant-2wtH) can be infused from incoming port 203. Droplets or hydrogel particles can be collected through port 204.

FIG. 3 illustrates an example of barcoding scheme by combinatorial split-and-pool barcoding. “T” represents a barcode-accepting polynucleotide. A pool of hardened particles are pooled first and separated into different parallel reactions. In one of the reaction, a first barcode segment A1 is attached to T. In a different parallel reaction, a first barcode segment A2 is attached to T. After barcoding the first segment, the hardened particles are pooled and separated into different parallel reactions again. A second segment is then attached to the first segment. The process can be repeated multiple times to generate a barcode with multiple segments. The combined segments form a complete barcode.

FIG. 4A illustrates an example construct of barcode-containing segments. A barcode-containing segment (401) can comprise 3A-a* which is complementary to a portion of the barcode-accepting polynucleotide, BC-a* which is a barcode sequence, and 3A-b* containing deoxyU nucleotides. The USER enzyme mix (e.g. from NEB) can be added to cut the deoxyU nucleotide after the barcode-containing segment is added onto the target. The barcode-containing segment can be modified on the 3′ end (403). Another barcode-containing segment containing a different barcode sequence is also provided (402). See Example 2 for further description.

FIG. 4B illustrates an example of barcoding strategy. A first barcode-containing segment hybridizes to the barcode-accepting polynucleotide (at the barcode acceptor site 3A-a) and the polynucleotide is extended. The USER enzyme mix is used to cut the deoxyU containing region to expose another barcode acceptor site (3A-b). The second barcode-containing segment is then added following the same process. See Example 2 for further description.

FIG. 4C illustrates an example of an immobilizable probe. In this example, the immobilizable probe is a polynucleotide probe with an immobilization moiety X. The polynucleotide probe comprises a DA region and a RTP region. The immobilizable probe can be an Acrydite-modified polyT-based RT primer having the following sequence: /5Acryd/CGACATCTAA CCTAGCTC TTTTTTTTTT TTTTTTTTTT TTTTTTTTTTV (SEQ ID NO: 3), where /5Acryd/ is the 5′ Acrydite modification. The first 18 nt (′CGACATCTAA CCTAGCTC′ (SEQ ID NO: 4)) provides a primer binding site for downstream processing. This domain can be called Domain A (or DA) throughout this disclosure. The RTP region is the reverse transcription primer. For example, this probe can be used to capture mRNA.

FIG. 4D illustrates another example of an immobilizable probe. In this example, the immobilizable probe comprises two polynucleotide strands. The strand containing RTP can be the targeting moiety which functions to hybridize with a target polynucleotide. The hybridization between DA and DA* is the linkage between the targeting moiety and immobilization moiety. In this example, the barcode can be added to 5A-a region which functions as barcode acceptor site.

FIG. 5A illustrates a general structure of an immobilizable probe. The immobilizable probe comprises a targeting moiety Y and an immobilization moiety X.

FIG. 5B illustrates an example of an immobilizable probe functioning in a vessel to link a target (T) onto the polymer framework. A barcode is attached to a barcode-accepting polynucleotide. As shown here, a polymer framework (502) is immobilized with an immobilizable probe (X-Y) through immobilization moiety (X). The linkage (503) between the framework and X can be covalent or non-covalent. A target is linked to the targeting moiety (Y) of the immobilizable probe. The linkage (504) between T and Y can be a covalent bond, an affinity binding, or nucleic acid hybridization. A barcode-accepting polynucleotide (505) is linked to the target or the immobilizable probe. It is to be understood that the barcode-accepting polynucleotide can be directly or indirectly linked to the target or the immobilizable probe. A barcode (506) is added or attached onto the barcode-accepting polynucleotide.

FIG. 6A illustrates an immobilization and barcoding scheme through affinity binding of a target (e.g. a protein, 602). In this example, the targeting moiety (Y, 601) can be an antibody which binds to a protein target. A detection probe (603) which is another antibody can be linked to a barcode-accepting polynucleotide (604) for barcode attachment.

FIG. 6B illustrates an immobilization and barcoding scheme of a target (e.g. methylated double-stranded DNA, 607). In this example, the targeting moiety (605) can be an antibody which binds to the methylated DNA. The target can serve as barcode-accepting polynucleotide.

FIG. 6C illustrates an immobilization and barcoding scheme of a target (e.g. a chromosomal DNA wrapped around histones, 611). In this example, the targeting moiety can be an antibody (609) that binds to the histone tail (610). The target can serve as a barcode-accepting polynucleotide.

FIG. 6D illustrates an immobilization and barcoding scheme of a target (e.g. an active enzyme, 615). This scheme can be used to perform a functional assay to select active enzymes. A suicide substrate (613) can be the targeting moiety of the immobilizable probe. When an active enzyme is bound to the suicide substrate, a covalent bound is formed between the suicide substrate and the enzyme to stably link the enzyme to the polymer framework. A detection probe (616) which can be an antibody specific for the enzyme is present to detect the enzyme. The detection probe is linked to a barcode-accepting polynucleotide (617) for downstream barcoding.

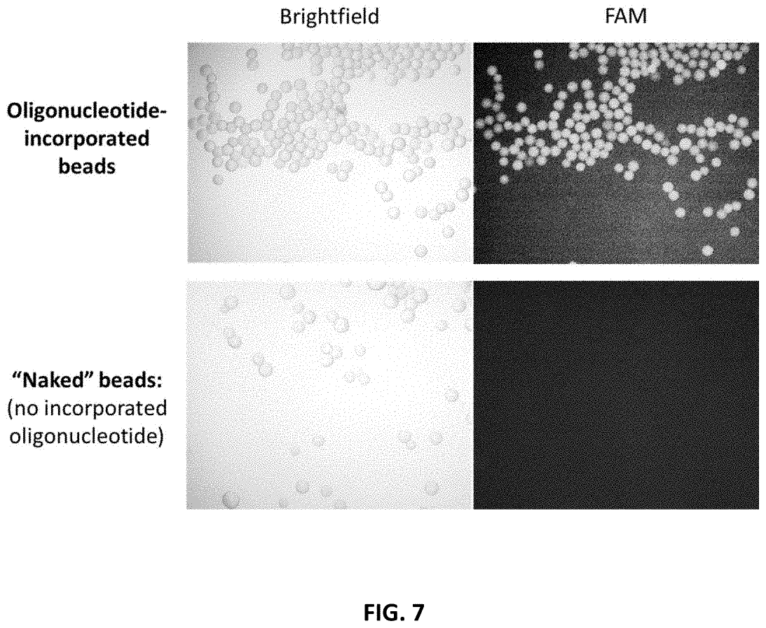

FIG. 7 illustrates images of polyacrylamide hydrogel particles incorporated with oligonucleotides that can function as the immobilizable probes (top panel). The incorporated oligonucleotides can be detected by detectable labels, e.g., FAM-labeled oligonucleotides that are complementary to the incorporated oligonucleotides. The hydrogel particles without any incorporated oligonucleotide cannot be detected by detectable labels, and therefore are invisible under FAM channel (bottom panel).

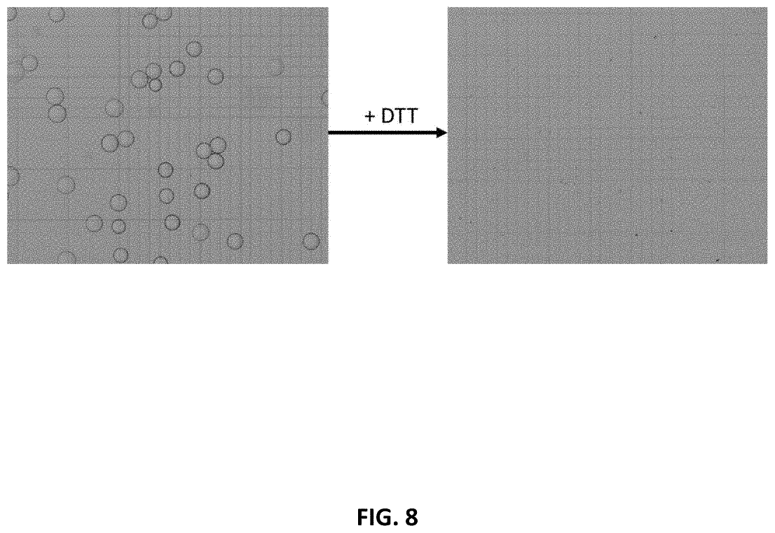

FIG. 8 illustrates an example of melting hydrogel particles using a reducing agent. In this example, DTT is used to melt polyacrylamide hydrogel particles.

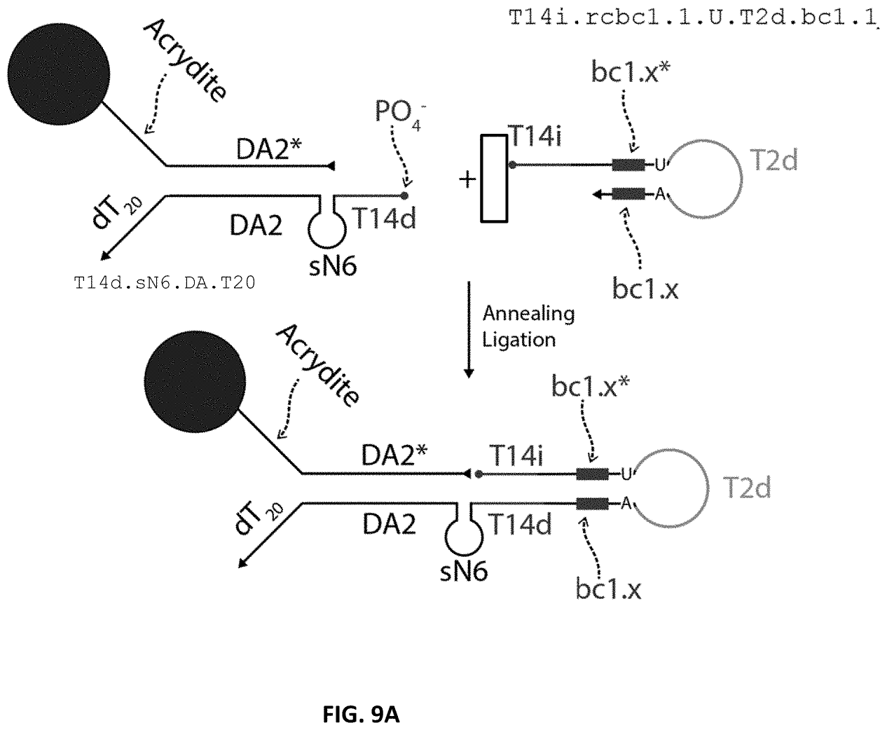

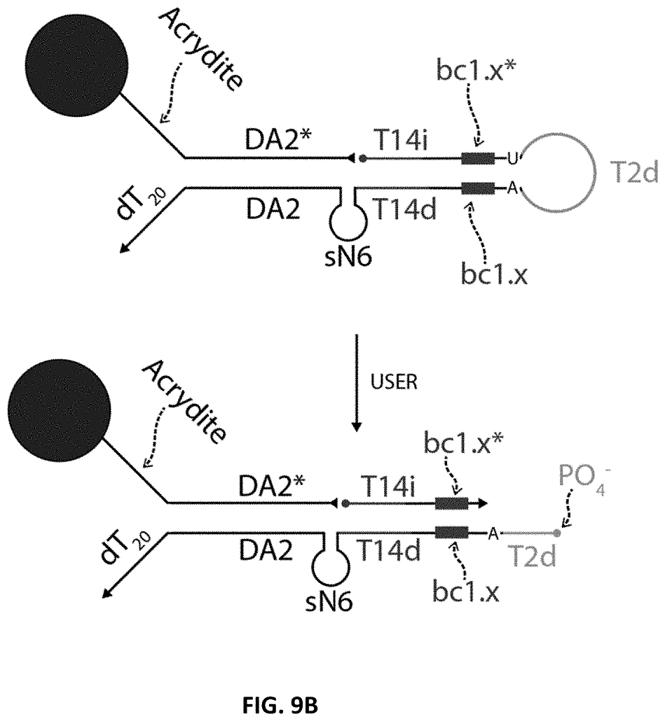

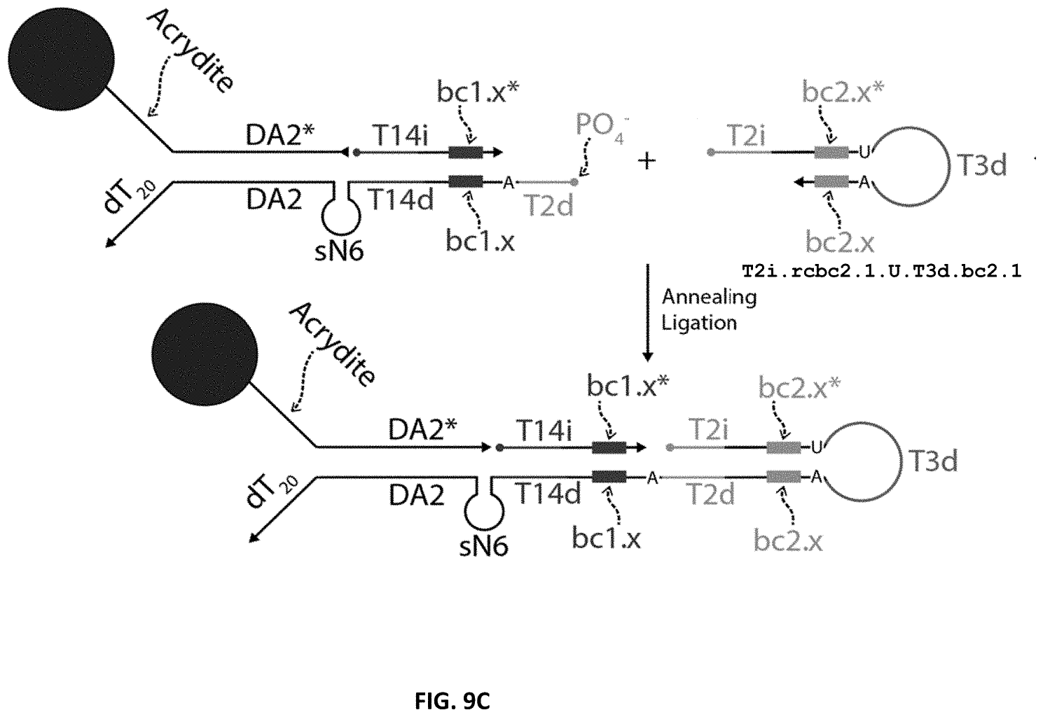

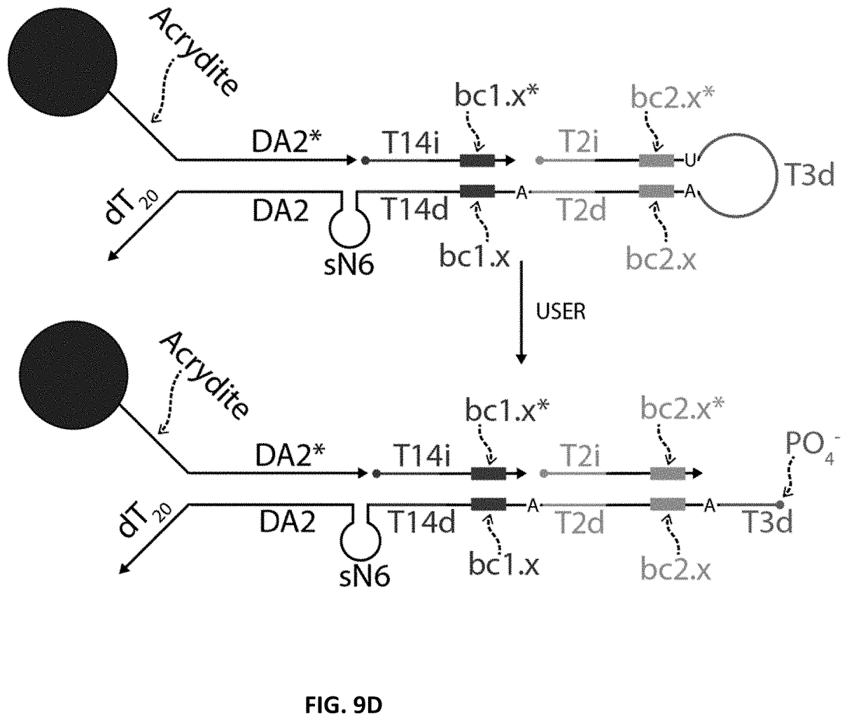

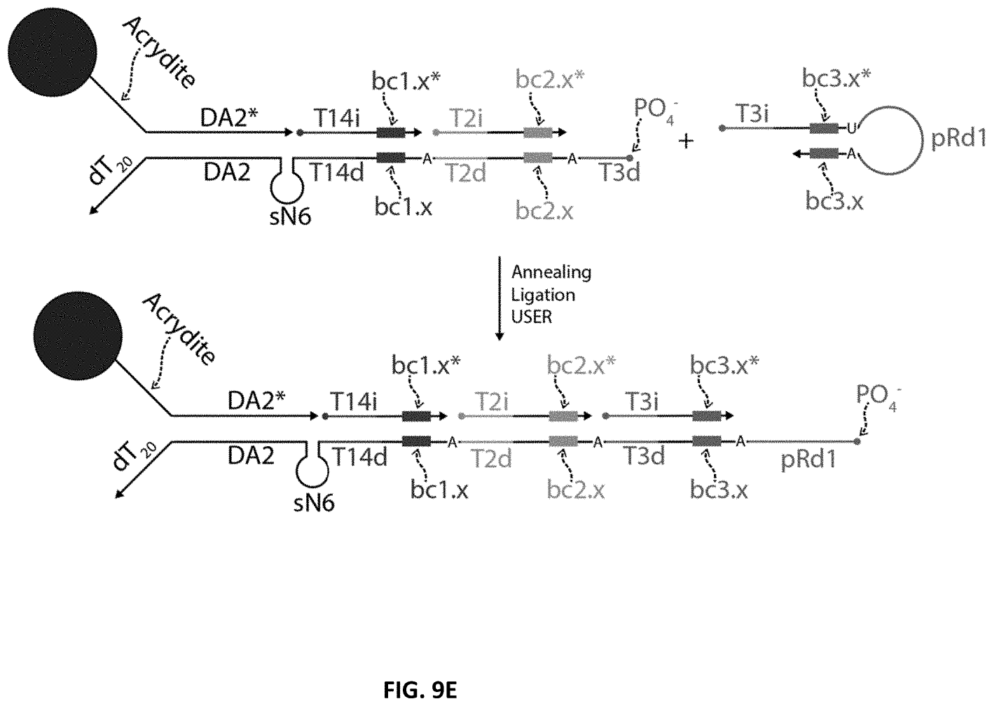

FIGS. 9A-9D illustrate an example scheme of ligating one or more barcodes (or barcode segments) onto an immobilizable probe entrapped in the hydrogel particle. The immobilizable probe has a barcode accepting polynucleotide to be ligated to the barcode. FIG. 9A illustrates ligating a barcode onto the barcode acceptor site of the barcode accepting polynucleotide. In this example, the barcode has a hairpin structure containing a deoxyuridine base. After ligating in FIG. 9A, the deoxyuridine base can be cleaved using the USER enzyme mix to generate another barcode acceptor site as illustrated in FIG. 9B. The schemes can be repeated for multiple rounds as illustrated in FIG. 9C-9E.

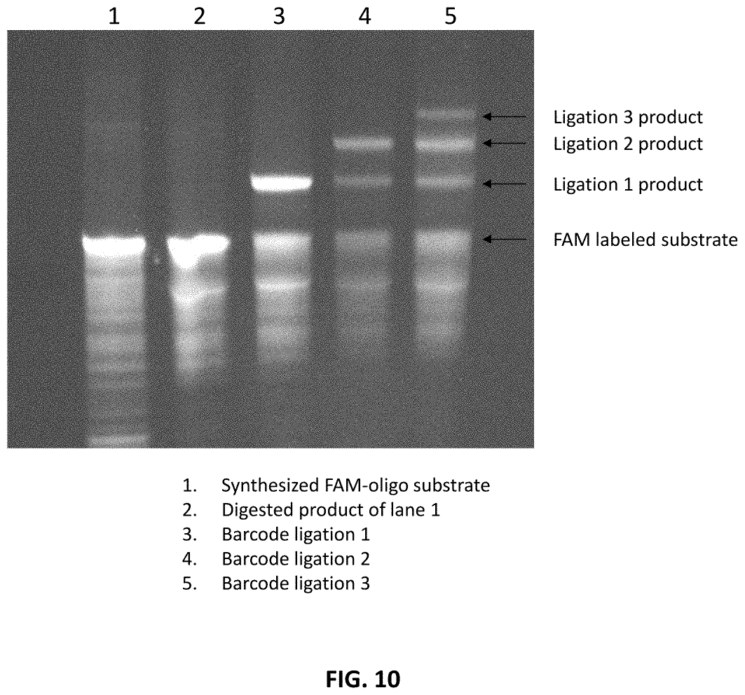

FIG. 10 illustrates an experimental data of generating barcodes using hardened particles. Using the schemes illustrated in FIGS. 9A-9E, immobilizable probes entrapped in the hydrogel particles can be ligated to one or more barcode sequences, resulting in ligation products with different length as shown in the gel image lanes 3-5.

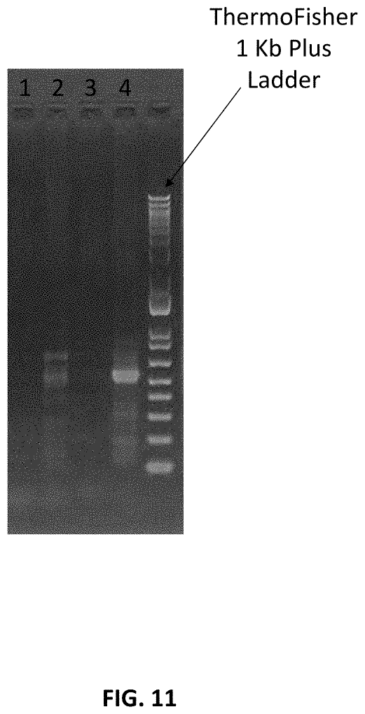

FIG. 11 illustrates an experimental data of entrapping target molecules in hardened particles. In this example, target mRNAs encoding TCR alpha subunit and TCR beta subunit are entrapped in the hydrogel particles using immobilizable probes having RT primers. To detect the entrapped target molecules, nucleic acid amplification were performed. In the gel image, lanes 2 and 4 show the entrapped mRNAs encoding TCR alpha subunit and TCR beta subunits, respectively. Lanes 1 and 3 show negative controls without entrapped target molecules.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EMBODIMENT PARAGRAPHS

[1] A method of barcoding comprising contacting a barcode to a hardened particle comprising polymerized or gelled polymers and/or monomers and a single cell, a single complex of cell, a single exosome, a target or derivative thereof, or a combination thereof and diffusing the barcode into the hardened particle.
[2] The method of paragraph [1], further comprising forming a compartment.
[3] The method of paragraph [2], comprising forming the compai linent before contacting the barcode.
[4] The method of paragraph [2] or [3], wherein the compartment comprises a plurality of polymerizable or gellable polymers and/or monomers; the single cell, the single complex of cells, the single exosome, the target, or a combination thereof; and an immobilizable probe.
[5] The method of any one of paragraphs [2]-[4], further comprising polymerizing or gelling the plurality of polymers and/or monomers in the compartment, thereby forming the hardened particle.
[6] The method of any one of paragraphs [1]-[5], wherein the polymers comprise polysaccharides, polyacrylamides, polyacrylic acids, polyethylene glycols, polyvinyl alcohols, polymethacrylamides, or any combination thereof[7] The method of paragraph [6], wherein the polysaccharides comprise agarose, hyaluronic acids, carboxymethycellose, chitosan, alginate, or any combination thereof[8] The method of any one of paragraphs [1]-[5], wherein the monomers comprise acrylic acids, acrylamides, methacrylamides, methacrylic acids, or any combination thereof[9] The method of any one of paragraphs [1]-[8], wherein the compartment or the hardened particle further comprises a crosslinking agent.
[10] The method of any one of paragraphs [1]-[9], wherein the compartment or the hardened particle further comprises a polymerization initiator.
[11] The method of paragraph [10], wherein the polymerization initiator is a photo-initiator.
[12] The method of paragraph [10], wherein the polymerization initiator is ammonium persulfate (APS), N,N,N′,N′-tetramethylethane-1,2-diamine (TEMED), Lithium- and magnesium phenyl-2,4,6-trimethylbenzoylphosphinates (TMPPL and TMPPM), sodium 4-[2-(4-morpholino)benzoyl-2-dimethylamino]-butylbenzenesulfonate (MBS), methylated-O-cyclodextrin (MI3CD), or 2,2-dimethoxy-2-phenyl acetophenone (DMPA), or any combination thereof.
[13] The method of any one of paragraphs [1]-[12], further comprising removing a thin liquid layer surrounding the hardened particle.
[14] The method of any one of paragraphs [1]-[13], wherein the single cell, the single complex of cells, or the single exosome is lysed.
[15] The method of any one of paragraphs [5]-[13], further comprising lysing the single cell, the single complex of cells, or the single exosome before or after polymerizing or gelling the plurality of polymers and/or monomers.
[16] The method of any one of paragraphs [1]-[14], wherein the target or derivative thereof is from the single cell, the single complex of cells, or the single exosome.
[17] The method of any one of paragraphs [4]-[16], wherein the target or derivative thereof is bound to the immobilizable probe.
[18] The method of any one of paragraphs [1]-[17], wherein the target is a target polynucleotide, a target polypeptide, a target organelle, or any combination thereof.
[19] The method of paragraph [18], wherein the target polynucleotide is a target deoxyribonucleic acid (DNA), a target ribonucleic acid (RNA), or a combination thereof.
[20] The method of paragraph [19], wherein the target DNA is a genomic DNA, a modified DNA, or a combination thereof.
[21] The method of paragraph [20], wherein the modified DNA is a methylated DNA.
[22] The method of paragraph [19], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[23] The method of any one of paragraphs [19]-[22], wherein the target is a target polynucleotide and the derivative of the target is a complementary strand of the target polynucleotide.
[24] The method of paragraph [23], wherein the complementary strand is a cDNA strand or a templated-switched cDNA strand.
[25] The method of paragraph [18], wherein the target polypeptide is a peptide, a protein or a portion thereof, a modified protein or a portion thereof, a secreted protein, a protein complex, or any combination thereof.
[26] The method of any one of paragraphs [4]-[25], wherein the immobilizable probe comprises a targeting moiety, which targeting moiety links the immobilizable probe to the target or derivative thereof.
[27] The method of paragraph [26], wherein the targeting moiety is a polynucleotide, a polypeptide, or a chemical group.
[28] The method of paragraph [26], wherein the target is a target polynucleotide and the targeting moiety is a primer.
[29] The method of paragraph [28], wherein the primer is a reverse transcription primer.
[30] The method of paragraph [29], wherein the reverse transcription primer comprises a poly-deoxy-thymidine nucleotide sequence.
[31] The method of any one of paragraphs [28]-[30], further comprising annealing the primer to the target polynucleotide.
[32] The method of any one of paragraphs [28]-[31], further comprising extending the primer to generate a complementary strand of the target polynucleotide.
[33] The method of paragraph [32], wherein the extending is performed before or after the polymerizing or gelling of the polymers and/or monomers.
[34] The method of paragraph [27], wherein the polypeptide is an antibody or a fragment thereof, or a peptide aptamer.
[35] The method of paragraph [27], wherein the chemical group forms a covalent bond with an amino acid side chain or a nucleobase of the target.
[36] The method of paragraph [27] or [35], wherein the chemical group is an N-hydroxysuccinimide (NHS) ester, a maleimide group, a hydrazide group, an azide group, or Label-IT linker and reactive group.
[37] The method of paragraph [35] or [36], wherein the amino acid side chain comprises an amine group, a carboxylate group, a carbonyl group, or a sulfhydryl group.
[38] The method of any one of paragraphs [35]-[37], wherein the nucleobase is guanine, adenine, cytosine or thymine.
[39] The method of any one of paragraphs [4]-[38], wherein the immobilizable probe comprises an immobilization moiety, which immobilization moiety links the immobilizable probe to one or more of the polymers and/or monomers of the polymerized or gelled plurality.
[40] The method of paragraph [39], wherein the immobilization moiety wherein the immobilization moiety is a chemical that is co-polymerized with the polymers and/or monomers, a chemical or protein that stably interact with the polymers and/or monomers directly, or a chemical or protein that stably interacts with the polymers and/or monomers indirectly.
[41] The method of any one of paragraphs [39]-[40], wherein the immobilization moiety is an NHS ester, a biotin, a maleimide group, a thiol group, an azide group, an avidin or streptavidin, a single-stranded polynucleotide, a biotin group, or a methacryl group.
[42] The method of any one of paragraphs [4]-[41], further comprising attaching the barcode to a barcode-accepting polynucleotide, wherein the barcode-accepting polynucleotide is the immobilizable probe or the target or derivative thereof.
[43] The method of any one of paragraphs [1]-[41], wherein the compartment or the hardened particle further comprises a detection probe, which detection probe binds to the target or derivative thereof.
[44] The method of paragraph [43], wherein the barcode is attached to the detection probe.
[45] The method of any one of paragraphs [1]-[44], wherein the barcode has a size that is smaller than a pore size of the hardened particle.
[46] The method of any one of paragraphs [1]-[45], wherein the barcode is from 5 to 500 nucleotides in length.
[47] The method of any one of paragraphs [1]-[46], wherein the hardened particle is a plurality of hardened particles.
[48] The method of paragraph [47], wherein attaching the barcode within the plurality of hardened particles comprises: pooling the plurality of hardened particles; dividing the plurality of hardened particles into separate first populations; attaching a first oligonucleotide comprising a first barcode sequence segment to the separate first populations, wherein each separate first population comprises a different first barcode sequence segment; pooling the separate first populations to provide a first pooled population; separating the first pooled population into a plurality of second populations; attaching a second oligonucleotide comprising a second barcode sequence segment to the first oligonucleotide already attached to the second populations, wherein each of the plurality of second populations comprises a different second barcode sequence segment; and pooling the plurality of second populations to provide a second pooled population comprising a barcode library.
[49] The method of paragraph [48], wherein the first barcode sequence segment or the second barcode sequence segment is at least 1 nucleotide in length.
[50] The method of paragraph [48], wherein the first barcode sequence segment or the second barcode sequence segment is at least 4 nucleotides in length.
[51] The method of paragraph [48], wherein the first barcode sequence segment or the second barcode sequence segment is from about 4 to about 20 nucleotides in length.
[52] The method of any one of paragraphs [1]-[51], further comprising washing the hardened particle.
[53] A method of barcoding, comprising: generating a population of vessels comprising a first total number of vessels, each vessel of the population of vessels comprising (i) a target or derivative thereof from a single cell, a single complex of cells, or a single exosome; and (ii) a plurality of polymerizable or gellable polymers and/or monomers; polymerizing or gelling the plurality of polymerizable or gellable polymers and/or monomers of the population of vessels, thereby forming a population of hardened particles; and contacting a barcode to the population of hardened particles, thereby forming a population of barcoded hardened particles comprising a second total number of barcoded hardened particles, each barcoded hardened particle of the population of barcoded hardened particles comprising (i) the target or derivative thereof from the single cell, the single complex of cells, or the single exosome; and (ii) polymerized or gelled polymers/monomers; wherein the second total number is at least 90%, 91%, 92%, 93%, 94%, 95%, 96%, 97%, 98%, 99%, or at least 100% of the first total number.
[54] The method of paragraph [53], wherein the barcode diffuses into the hardened particles.
[55] The method of paragraph [53] or [54], further comprising removing a thin liquid layer surrounding the population of vessels.
[56] The method of any one of paragraphs [53]-[55], wherein the vessel or the hardened particle further comprises a non-cellular polynucleotide.
[57] A composition comprising a plurality of vessels, each vessel of the plurality comprising a hydrogel particle or a bead; an immobilizable probe embedded within the hydrogel particle or bead, wherein the immobilizable probe is immobilized to a diffusion restricting agent; and a target or derivative thereof embedded within the hydrogel particle or bead and linked to the immobilizable probe, wherein the target is from a single cell, a single complex of cells, or a single exosome, or the derivative thereof is a derivative of a target from a single cell, a single complex of cells, or a single exosome; wherein the hydrogel particle or the bead has a volume that is at least 50% of a volume of the vessel.
[58] The composition of paragraph [57], wherein the target is from a different single cell, a different single complex of cells, or a different single exosome.
[59] The composition of paragraph [57], wherein the hydrogel particle or the bead has a volume that is at least 70%, 80%, or 90% of the volume of the vessel.
[60] The composition of paragraph [57], wherein the hydrogel particle or bead comprises a polymerized or gelled plurality of polymers and/or monomers.
[61] The composition of paragraph [60], wherein the polymers comprise polysaccharides, polyacrylamides, polyacrylic acids, polyethylene glycols, polyvinyl alcohols, polymethacrylamides, or any combination thereof.
[62] The composition of paragraph [61], wherein the polysaccharides comprise agarose, hyaluronic acids, carboxymethycellose, chitosan, alginate, or any combination thereof.
[63] The composition of paragraph [60], wherein the monomers comprise acrylic acids, acrylamides, methacrylamides, methacrylic acids, or any combination thereof.
[64] The composition of any one of paragraphs [57]-[63], wherein the immobilizable probe and the target or derivative thereof are linked by a covalent bond or a non-covalent interaction.
[65] The composition of any one of paragraphs [57]-[64], wherein the immobilizable probe comprises an immobilization moiety, which immobilization moiety links the immobilizable probe to one or more polymers and/or monomers of the polymerized or gelled plurality.
[66] The composition of paragraph [65], wherein the immobilization moiety is a chemical that is co-polymerized with the polymers and/or monomers, a chemical or protein that stably interacts with the polymers and/or monomers directly, or a chemical or protein that stably interacts with the polymers and/or monomers indirectly.
[67] The composition of paragraph [65] or [66], wherein the immobilization moiety is an NHS ester, a biotin, a maleimide group, a thiol group, an azide group, an avidin or streptavidin, a single-stranded polynucleotide, a biotin group, or a methacryl group.
[68] The composition of any one of paragraphs [57]-[67], wherein the diffusion restricting agent is one or more polymers and/or monomers of the polymerized or gelled plurality.
[69] The composition of any one of paragraphs [57]-[67], wherein the diffusion restricting agent is a polymer chain conjugated on the immobilizable probe.
[70] The composition of any one of paragraphs [57]-[69], wherein the target is a target polynucleotide, a target polypeptide, a target organelle, or any combination thereof.
[71] The composition of paragraph [70], wherein the target polynucleotide is a target deoxyribonucleic acid (DNA), a target ribonucleic acid (RNA), or a combination thereof.
[72] The composition of paragraph [71], wherein the target DNA is a genomic DNA, a modified DNA, or a combination thereof.
[73] The composition of paragraph [71], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[74] The composition of any one of paragraphs [57]-[73], wherein the target is a target polynucleotide and the derivative of the target is a complementary strand of the target polynucleotide.
[75] The composition of paragraph [74], wherein the complementary strand is a cDNA strand or a template-switched cDNA strand.
[76] The composition of paragraph [70], wherein the target polypeptide is a peptide, a protein or a portion thereof, a modified protein or a portion thereof, a secreted protein, a protein complex, or any combination thereof.
[77] The composition of any one of paragraphs [57]-[76], wherein the immobilizable probe further comprises a targeting moiety, which targeting moiety links the immobilizable probe to the target or derivative thereof.
[78] The composition of paragraph [77], wherein the targeting moiety is a polynucleotide, a polypeptide, or a chemical group.
[79] The composition of paragraph [78], wherein the polynucleotide is a primer or an oligonucleotide aptamer.
[80] The composition of paragraph [79], wherein the primer is a reverse transcription primer.
[81] The composition of paragraph [80], wherein the reverse transcription primer comprises a poly-deoxy-thymidine nucleotide sequence.
[82] The composition of paragraph [78], wherein the polypeptide is an antibody or a fragment thereof, or a peptide aptamer.
[83] The composition of paragraph [78], wherein the chemical group forms a covalent bond with an amino acid side chain or a nucleobase of the target.
[84] The composition of paragraph [83], wherein the chemical group is an N-hydroxysuccinimide (NHS) ester, a maleimide group, a hydrazide group, an azide group or Label-IT linker and reactive group.
[85] The composition of paragraph [83] or [84], wherein the amino acid side chain comprises an amine group, a carboxylate group, a carbonyl group, or a sulfhydryl group.
[86] The composition of any one of paragraphs [83]-[85], wherein the nucleobase is guanine, adenine, cytosine or thymine.
[87] The composition of any one of paragraphs [57]-[86], wherein each vessel of the plurality further comprises a lysing agent.
[88] The composition of paragraph [87], wherein the lysing agent is a cell lysing agent.
[89] The composition of any one of paragraphs [57]-[87], wherein the different single cell, the different single complex of cells, or the different single exosome is lysed.
[90] The composition of paragraph [57]-[64], wherein the derivative is a copied product of the target.
[91] The composition of paragraph [90], wherein the copied product of the target is linked to the immobilizable probe via a phosphodiester bond.
[92] A composition comprising a plurality of hydrogel particles or beads, each hydrogel particle or bead of the plurality comprising an immobilizable probe embedded within the hydrogel particle or the bead, wherein the immobilizable probe is immobilized to a diffusion restricting agent or has a hydrodynamic radius that is larger than a pore size of the hydrogel particle or the bead; a target or derivative thereof embedded within the hydrogel particle or the bead and linked to the immobilizable probe, wherein the target is from a single cell, a single complex of cells, or a single exosome or the derivative thereof is a derivative of a target from a single cell, a single complex of cells, or a single exosome; and a barcode.
[93] The composition of paragraph [92], wherein each of hydrogel particle or bead comprises a polymerized or gelled plurality of polymers and/or monomers.
[94] The composition of paragraph [92]-[93], wherein the diffusion restricting agent is one or more polymers and/or monomers of the polymerized or gelled plurality.
[95] The composition of any one of paragraphs [92]-[94], wherein the diffusion restricting agent is a polymer chain conjugated on the immobilizable probe.
[96] The composition of paragraph [95], wherein the polymer chain is a polyethylene glycol molecule or a polyacrylamide molecule.
[97] The composition of any one of paragraphs [92]-[96], wherein the immobilizable probe comprises an immobilization moiety, which immobilization moiety links the immobilizable probe to one or more polymers and/or monomers of the polymerized or gelled plurality.
[98] The composition of paragraph [97], wherein the immobilization moiety is a chemical that is co-polymerized with the polymers and/or monomers, a chemical or protein that stably interacts with the polymers and/or monomers directly, or a chemical or protein that stably interacts with the polymers and/or monomers indirectly.
[99] The composition of paragraph [98], wherein the immobilization moiety is an NHS ester, a biotin, a maleimide group, a thiol group, an azide group, an avidin or streptavidin, a single-stranded polynucleotide, a biotin group, or a methacryl group.
[100] The composition of any one of paragraphs [92]-[99], wherein the target is a target polynucleotide, a target polypeptide, a target organelle, or any combination thereof.
[101] The composition of paragraph [100], wherein the target polynucleotide is a target deoxyribonucleic acid (DNA), a target ribonucleic acid (RNA), or a combination thereof.
[102] The composition of paragraph [101], wherein the target DNA is a genomic DNA, a modified DNA, or a combination thereof.
[103] The composition of paragraph [101], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[104] The composition of any one of paragraphs [100]-[103], wherein the target is a target polynucleotide and the derivative of the target is a complementary strand of the target polynucleotide.
[105] The composition of paragraph [104], wherein the complementary strand is a cDNA strand or a template-switched cDNA strand.
[106] The composition of paragraph [100], wherein the target polypeptide is a peptide, a protein or a portion thereof, a modified protein or a portion thereof, a secreted protein, a protein complex, or any combination thereof.
[107] The composition of any one of paragraphs [92]-[106], wherein the immobilizable probe further comprises a targeting moiety, which targeting moiety links the immobilizable probe to the target or derivative thereof.
[108] The composition of paragraph [107], wherein the targeting moiety is a polynucleotide, a polypeptide, or a chemical group.
[109] The composition of paragraph [108], wherein the polynucleotide is a primer or an oligonucleotide aptamer.
[110] The composition of paragraph [109], wherein the primer is a reverse transcription primer.
[111] The composition of paragraph [110], wherein the reverse transcription primer comprises a poly-deoxy-thymidine nucleotide sequence.
[112] The composition of paragraph [108], wherein the polypeptide is an antibody or a fragment thereof, or a peptide aptamer.
[113] The composition of paragraph [108], wherein the chemical group forms a covalent bond with an amino acid side chain or a nucleobase of the target.
[114] The composition of paragraph [108] or [113], wherein the chemical group is an N-hydroxysuccinimide (NHS) ester, a maleimide group, a hydrazide group, an azide group or Label-IT linker and reactive group.
[115] The composition of paragraph [113] or [114], wherein the amino acid side chain comprises an amine group, a carboxylate group, a carbonyl group, or a sulfhydryl group.
[116] The composition of any one of paragraphs [113]-[115], wherein the nucleobase is guanine, adenine, cytosine or thymine.
[117] The composition of any one of paragraphs [92]-[116], wherein the different single cell, the different single complex of cells, or the different single exosome is lysed.
[118] The composition of any one of paragraphs [92]-[117], wherein the barcode is attached to the target or derivative thereof or the immobilizable probe.
[119] The composition of any one of paragraphs [92]-[118], wherein the barcode is from 5 to 500 nucleotides in length.
[120] A hardened particle comprising a polymerized or gelled plurality of polymers and/or monomers; a single cell, a single complex of cells, or a single exosome; an immobilizable probe linked to one or more polymers and/or monomers of the polymerized or gelled plurality; and a barcode.
[121] The hardened particle of paragraph [120], wherein the hardened particle is a hydrogel particle.
[122] The hardened particle of paragraph [120] or [121], wherein the immobilizable probe comprises an immobilization moiety, which immobilization moiety links the immobilization probe to one or more polymers and/or monomers of the polymerized or gelled plurality.
[123] The hardened particle of paragraph [122] or [122], wherein the immobilization moiety is a chemical that is co-polymerized with the polymers and/or monomers, a chemical or protein that stably interacts with the polymers and/or monomers directly, or a chemical or protein that stably interacts with the polymers and/or monomers indirectly.
[124] The hardened particle of any one of paragraphs [122]-[123], wherein the immobilization moiety is an NHS ester, a biotin, a maleimide group, a thiol group, an azide group, an avidin or streptavidin, a single-stranded polynucleotide, a biotin group, or a methacryl group.
[125] A hardened particle comprising: a polymerized or gelled plurality of polymers and/or monomers; a single cell, a single complex of cells, or a single exosome; an immobilizable probe, wherein the immobilizable probe has a hydrodynamic radius that is larger than a pore size of the vessel; and a barcode.
[126] The hardened particle of paragraph [125], wherein the immobilizable probe comprises an immobilization moiety.
[127] The hardened particle of paragraph [126], wherein the immobilization moiety is conjugated to a polymer chain.
[128] The hardened particle of paragraph [127], wherein the immobilization polymer is a polyethylene glycol molecule.
[129] The hardened particle of any one of paragraphs [125]-[128], further comprising a barcode.
[130] The hardened particle of any one of paragraphs [120]-[128], further comprising a target from the single cell, the single complex of cell, or the single exosome.
[131] The hardened particle of paragraph [130], wherein the target is bound to the immobilizable probe.
[132] The hardened particle of paragraph [130] or [131], wherein the target is a target polynucleotide, a target polypeptide, a target organelle, or any combination thereof.
[133] The hardened particle of paragraph [132], wherein the target polynucleotide is a target deoxyribonucleic acid (DNA), a target ribonucleic acid (RNA), or a combination thereof.
[134] The hardened particle of paragraph [133], wherein the target DNA is a genomic DNA, a modified DNA, or a combination thereof.
[135] The hardened particle of paragraph [134], wherein the modified DNA is a methylated DNA.
[136] The hardened particle of paragraph [133], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[137] The hardened particle of any one of paragraphs [133]-[136], further comprising a complementary strand of the target polynucleotide.
[138] The hardened particle of paragraph [137], wherein the complementary strand is a cDNA strand.
[139] The hardened particle of paragraph [132], wherein the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof.
[140] The hardened particle of paragraph [132], wherein the target polypeptide is a peptide, a protein or a portion thereof, a modified protein or a portion thereof, a protein complex, or any combination thereof.
[141] The hardened particle of any one of paragraphs [120]-[140], wherein the immobilizable probe further comprises a targeting moiety.
[142] The hardened particle of paragraph [141], wherein the targeting moiety is a polynucleotide, a polypeptide, or a chemical group.
[143] The hardened particle of paragraph [142], wherein the polynucleotide is a primer or an oligonucleotide aptamer.
[144] The hardened particle of paragraph [143], wherein the primer is a reverse transcription primer.
[145] The hardened particle of paragraph [144], wherein the reverse transcription primer comprises a poly-deoxy-thymidine nucleotide sequence.
[146] The hardened particle of paragraph [142], wherein the polypeptide is an antibody or a fragment thereof, or a peptide aptamer.
[147] The hardened particle of paragraph [142], wherein the chemical group is a reactive group.
[148] The hardened particle of paragraph [147], wherein the reactive group forms a covalent bond with an amino acid side chain or a nucleobase of the target.
[149] The hardened particle of paragraph [147] or [148], wherein the reactive group is an N-hydroxysuccinimide (NHS) ester, a maleimide group, a hydrazide group, an azide group or Label-IT linker and reactive group.
[150] The hardened particle of paragraph [148] or [149], wherein the amino acid side chain is lysine or cysteine side chain.
[151] The hardened particle of any one of paragraphs [148]-[150], wherein the amino acid side chain comprises an amine group, a carboxylate group, a carbonyl group, or a sulfhydryl group.
[152] The hardened particle of any one of paragraphs [148]-[151], wherein the nucleobase is guanine, adenine, cytosine or thymine.
[153] The hardened particle of any one of paragraphs [120]-[152], wherein the barcode is linked to the immobilizable probe, the target, or a complementary strand of the target polynucleotide.
[154] The hardened particle of any one of paragraphs [120]-[153], further comprising a detection probe.
[155] The hardened particle of paragraph [154], wherein the barcode is linked to the detection probe.
[156] The hardened particle of any one of paragraphs [120]-[155], wherein the barcode is from 5 to 500 nucleotides in length.
[157] The hardened particle of any one of paragraphs [120]-[156], wherein the polymers are polysaccharides or polyacrylamides.
[158] The hardened particle of paragraph [157], wherein the polysaccharides are poly-agarobioses, hyaluronic acids, carboxymethycellose, chitosan or alginate.
[159] The hardened particle of any one of paragraphs [120]-[158], wherein the monomers are acrylamide or methacrylamide monomers.
[160] The hardened particle of any one of paragraphs [120]-[159], wherein the polymerized or gelled plurality of polymers and/or monomers are cross-linked.
[161] A droplet, comprising: a single cell, a single complex of cells, or a single exosome; a plurality of polymerizable or gellable polymers and/or monomers; an immobilizable probe linked to one or more polymers and/or monomers of the plurality; and a non-cellular polynucleotide; wherein the non-cellular polynucleotide is linked to the immobilizable probe.
[162] The droplet of paragraph [161], wherein the single cell, the single complex of cells, or the single exosome is lysed.
[163] The droplet of paragraph [161] or [162], wherein the immobilizable probe comprises an immobilization moiety.
[164] The droplet of paragraph [163], wherein the immobilization moiety links the immobilization probe to one or more polymerizable or gellable polymers and/or monomers of the plurality.
[165] The droplet of paragraph [163] or [164], wherein the immobilization moiety is a chemical that is co-polymerized with the polymers and/or monomers, a chemical or protein that stably interacts with the polymers and/or monomers directly, or a chemical or protein that stably interacts with the polymers and/or monomers indirectly.
[166] The droplet of any one of paragraphs [163]-[165], wherein the immobilization moiety is an NHS ester, a biotin, a maleimide group, a thiol group, an azide group, an avidin or streptavidin, a single-stranded polynucleotide, a biotin group, or a methacryl group.
[167] The droplet of any one of paragraphs [161]-[166], further comprising a target from the single cell, the single complex of cell, or the single exosome.
[168] The droplet of paragraph [167], wherein the target is bound to the immobilizable probe.
[169] The droplet of paragraph [167] or [168], wherein the target is a target polynucleotide, a target polypeptide, a target organelle, or any combination thereof.
[170] The droplet of paragraph [169], wherein the target polynucleotide is a target deoxyribonucleic acid (DNA), a target ribonucleic acid (RNA), or a combination thereof.
[171] The droplet of paragraph [170], wherein the target DNA is a genomic DNA, a modified DNA, or a combination thereof.
[172] The droplet of paragraph [171], wherein the modified DNA is a methylated DNA.
[173] The droplet of paragraph [170], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[174] The droplet of any one of paragraphs [169]-[173], further comprising a complementary strand of the target polynucleotide.
[175] The droplet of paragraph [174], wherein the complementary strand is a cDNA strand.
[176] The droplet of paragraph [169], wherein the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof.
[177] The droplet of paragraph [169], wherein the target polypeptide is a peptide, a protein or a portion thereof, a modified protein or a portion thereof, a protein complex, or any combination thereof.
[178] The droplet of any one of paragraphs [161]-[177], wherein the immobilizable probe further comprises a targeting moiety.
[179] The droplet of paragraph [178], wherein the targeting moiety is a polynucleotide, a polypeptide, or a chemical group.
[180] The droplet of paragraph [179], wherein the polynucleotide is a primer or an oligonucleotide aptamer.
[181] The droplet of paragraph [180], wherein the primer is a reverse transcription primer.
[182] The droplet of paragraph [181], wherein the reverse transcription primer comprises a poly-deoxy-thymidine nucleotide sequence.
[183] The droplet of paragraph [179], wherein the polypeptide is an antibody or a fragment thereof, or a peptide aptamer.
[184] The droplet of paragraph [179], wherein the chemical group is a reactive group.
[185] The droplet of paragraph [184], wherein the reactive group forms a covalent bond with an amino acid side chain or a nucleobase of the target.
[186] The droplet of paragraph [184]-[185], wherein the reactive group is an N-hydroxysuccinimide (NHS) ester, a maleimide group, a hydrazide group, an azide group or Label-IT linker and reactive group.
[187] The droplet of paragraph [185] or [186], wherein the amino acid side chain is lysine or cysteine side chain.
[188] The droplet of any one of paragraphs [185]-[187], wherein the amino acid side chain comprises an amine group, a carboxylate group, a carbonyl group, or a sulfhydryl group.
[189] The droplet of any one of paragraphs [185]-[188], wherein the nucleobase is guanine, adenine, cytosine or thymine.
[190] The droplet of any one of paragraphs [161]-[189], wherein the droplet is a water-in-oil droplet.
[191] A method, comprising: generating a population of vessels, each comprising a plurality of polymerizable or gellable polymers or monomers, wherein a subpopulation of the population comprises (i) a target from a single cell, a single complex of cells or a single exosome, and (ii) a non-cellular polynucleotide; and polymerizing or gelling the plurality of polymerizable or gellable polymers or monomers of the population, wherein each vessel of the subpopulation comprises a hardened particle.
[192] The method of paragraph [191], further comprising contacting a barcode to the hardened particle.
[193] The method of paragraph [192], wherein the barcode diffuses into the hardened particle.
[194] The method of any one of paragraphs [191]-[193], further comprising removing a thin liquid layer surrounding the hardened particle.
[195] The method any one of paragraphs [53]-[194], wherein the single cell, the single complex of cells, or the single exosome is lysed.
[196] The method of any one of paragraphs [53]-[195], wherein the target is from the single cell, the single complex of cells, or the single exosome.
[197] The method of any one of paragraphs [53]-[196], wherein the target is a target polynucleotide, a target polypeptide, a target organelle, or any combination thereof.
[198] The method of paragraph [197], wherein the target polynucleotide is a target deoxyribonucleic acid (DNA), a target ribonucleic acid (RNA), or a combination thereof.
[199] The method of paragraph [198], wherein the target DNA is a genomic DNA, a modified DNA, or a combination thereof.
[200] The method of paragraph [199], wherein the modified DNA is a methylated DNA.
[201] The method of paragraph [198], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[202] The method of any one of paragraphs [197]-[201], further comprising a complementary strand of the target polynucleotide.
[203] The method of paragraph [202], wherein the complementary strand is a cDNA strand.
[204] The method of paragraph [197], wherein the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof.
[205] The method of paragraph [197], wherein the target polypeptide is a peptide, a protein or a portion thereof, a modified protein or a portion thereof, a protein complex, or any combination thereof.
[206] The method of any one of paragraphs [53]-[205], wherein each of the vessels or hardened particles further comprises an immobilizable probe.
[207] The method of paragraph [206], wherein the immobilizable probe is linked to one or more of the plurality of polymerizable or gellable polymers or monomers.
[208] The method of paragraph [206] or [207], wherein the immobilizable probe comprises a targeting moiety.
[209] The method of paragraph [208], wherein the targeting moiety is a polynucleotide, a polypeptide, or a chemical group.
[210] The method of paragraph [209], wherein the polynucleotide is a primer or an oligonucleotide aptamer.
[211] The method of paragraph [210], wherein the primer is a reverse transcription primer.
[212] The method of paragraph [211], wherein the reverse transcription primer comprises a poly-deoxy-thymidine nucleotide sequence.
[213] The method of any one of paragraphs [210]-[212], further comprising annealing the primer to the target polynucleotide.
[214] The method of any one of paragraphs [210]-[213], further comprising extending the primer to generate a complementary strand of the target polynucleotide.
[215] The method of paragraph [214], wherein the extending is performed before or after the polymerizing or gelling of the polymers and/or monomers.
[216] The method of paragraph [209], wherein the polypeptide is an antibody or a fragment thereof, or a peptide aptamer.
[217] The method of paragraph [209], wherein the chemical group is a reactive group.
[218] The method of paragraph [217], wherein the reactive group forms a covalent bond with an amino acid side chain or a nucleobase of the target.
[219] The method of paragraph [217] or [218], wherein the reactive group is an N-hydroxysuccinimide (NHS) ester, a maleimide group, a hydrazide group, an azide group, or Label-IT linker and reactive group.
[220] The method of paragraph [218] or [219], wherein the amino acid side chain is lysine or cysteine side chain.
[221] The method of any one of paragraphs [218]-[220], wherein the amino acid side chain comprises an amine group, a carboxylate group, a carbonyl group, or a sulfhydryl group.
[222] The method of any one of paragraphs [218]-[221], wherein the nucleobase is guanine, adenine, cytosine or thymine.
[223] The method of any one of paragraphs [206]-[222], wherein the immobilizable probe comprises an immobilization moiety.
[224] The method of paragraph [223], wherein the immobilization moiety links the immobilizable probe to one or more of the polymers and/or monomers of the polymerized or gelled plurality.
[225] The method of paragraph [224], wherein the immobilization moiety a chemical that is co-polymerized with the polymers and/or monomers, a chemical or protein that stably interact with the polymers and/or monomers directly, or a chemical or protein that stably interacts with the polymers and/or monomers indirectly.
[226] The method of any one of paragraphs [223]-[225], wherein the immobilization moiety is an NHS ester, a biotin, a maleimide group, a thiol group, an azide group, an avidin or streptavidin, a single-stranded polynucleotide, a biotin group, or a methacryl group.
[227] The method of any one of paragraphs [53]-[226], wherein the barcode is attached to the immobilizable probe, the target, or the complementary strand of the target polynucleotide.
[228] The method of any one of paragraphs [56]-[227], wherein the non-cellular polynucleotide is a barcode-accepting polynucleotide.
[229] The method of any one of paragraphs [56]-[228], wherein the barcode is attached to the barcode-accepting polynucleotide.
[230] The method of any one of paragraphs [53]-[229], wherein the compartment or vessel further comprises a detection probe.
[231] The method of paragraph [230], wherein the barcode-accepting polynucleotide is attached to the detection probe.
[232] The method of any one of paragraphs [191]-[231], wherein the vessel is a water-in-oil droplet.
[233] The method of any one of paragraphs [191]-[232], wherein the hardened particle is a hydrogel particle.
[234] A method, comprising: generating a population of vessels, each comprising a plurality of polymerizable or gellable polymers or monomers, wherein a subpopulation of the population comprises a single cell, a single complex of cells or a single exosome; polymerizing or gelling the plurality of polymerizable or gellable polymers or monomers of the population, wherein each vessel of the subpopulation comprises a hardened particle; and lysing the single cell, the single complex of cell, or the single exosome.
[235] The method of paragraph [234], further comprising removing a thin liquid layer surrounding the hardened particle.
[236] The method of paragraph [234] or [235], further comprising contacting a barcode to the hardened particle.
[237] The method of paragraph [236], wherein the barcode diffuses into the hardened particle.
[238] The method of any one of paragraphs [234]-[237], wherein the vessel or hardened particle further comprises a target from the single cell, the single complex of cells, or the single exosome.
[239] The method of paragraph [238], wherein the target is a target polynucleotide, a target polypeptide, a target organelle, or any combination thereof.
[240] The method of paragraph [239], wherein the target polynucleotide is a target deoxyribonucleic acid (DNA), a target ribonucleic acid (RNA), or a combination thereof.
[241] The method of paragraph [240], wherein the target DNA is a genomic DNA, a modified DNA, or a combination thereof.
[242] The method of paragraph [241], wherein the modified DNA is a methylated DNA.
[243] The method of paragraph [240], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[244] The method of any one of paragraphs [239]-[243], further comprising a complementary strand of the target polynucleotide.
[245] The method of paragraph [244], wherein the complementary strand is a cDNA strand.
[246] The method of paragraph [239], wherein the target organelle is acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof.
[247] The method of paragraph [239], wherein the target polypeptide is a peptide, a protein or a portion thereof, a modified protein or a portion thereof, a protein complex, or any combination thereof.
[248] The method of any one of paragraphs [234]-[247], wherein each of the vessels or hardened particles further comprises an immobilizable probe.
[249] The method of paragraph [248], wherein the immobilizable probe is linked to one or more of the plurality of polymerizable or gellable polymers or monomers.
[250] The method of paragraph [248] or [249], wherein the immobilizable probe comprises a targeting moiety.
[251] The method of paragraph [250], wherein the targeting moiety is a polynucleotide, a polypeptide, or a chemical group.
[252] The method of paragraph [251], wherein the polynucleotide is a primer or an oligonucleotide aptamer.
[253] The method of paragraph [252], wherein the primer is a reverse transcription primer.
[254] The method of paragraph [253], wherein the reverse transcription primer comprises a poly-deoxy-thymidine nucleotide sequence.
[255] The method of any one of paragraphs [252]-[254], further comprising annealing the primer to the target polynucleotide.
[256] The method of any one of paragraphs [252]-[255], further comprising extending the primer to generate a complementary strand of the target polynucleotide.
[257] The method of paragraph [256], wherein the extending is performed before or after the polymerizing or gelling of the polymers and/or monomers.
[258] The method of paragraph [251], wherein the polypeptide is an antibody or a fragment thereof, or a peptide aptamer.
[259] The method of paragraph [251], wherein the chemical group is a reactive group.
[260] The method of paragraph [259], wherein the reactive group forms a covalent bond with an amino acid side chain or a nucleobase of the target.
[261] The method of paragraph [259] or [260], wherein the reactive group is an N-hydroxysuccinimide (NHS) ester, a maleimide group, a hydrazide group, an azide group, or Label-IT linker and reactive group.
[262] The method of paragraph [260] or [261], wherein the amino acid side chain is lysine or cysteine side chain.
[263] The method of any one of paragraphs [260]-[262], wherein the amino acid side chain comprises an amine group, a carboxylate group, a carbonyl group, or a sulfhydryl group.
[264] The method of any one of paragraphs [260]-[263], wherein the nucleobase is guanine, adenine, cytosine or thymine.
[265] The method of any one of paragraphs [248]-[264], wherein the immobilizable probe comprises an immobilization moiety.
[266] The method of paragraph [265], wherein the immobilization moiety links the immobilizable probe to one or more of the polymers and/or monomers of the polymerized or gelled plurality.
[267] The method of paragraph [266], wherein the immobilization moiety a chemical that is co-polymerized with the polymers and/or monomers, a chemical or protein that stably interact with the polymers and/or monomers directly, or a chemical or protein that stably interacts with the polymers and/or monomers indirectly.
[268] The method of any one of paragraphs [265]-[267], wherein the immobilization moiety is an NHS ester, a biotin, a maleimide group, a thiol group, an azide group, an avidin or streptavidin, a single-stranded polynucleotide, a biotin group, or a methacryl group.
[269] The method of any one of paragraphs [236]-[268], wherein the barcode is attached to the immobilizable probe, the target, or the complementary strand of the target polynucleotide.
[270] The method of any one of paragraphs [234]-[269], wherein the compartment or vessel further comprises a detection probe.
[271] The method of paragraph [270], wherein the barcode is attached to the detection probe.
[272] The method of any one of paragraphs [53]-[271], further comprising washing the hardened particles extensively.
[273] The method any one of paragraphs [234]-[272], wherein the compartment or the vessel is a water-in-oil droplet.
[274] The method of any one of paragraphs [234]-[273], wherein the hardened particle is a hydrogel particle.
[275] A method of barcoding a target, comprising: forming a plurality of vessels each comprising: a plurality of polymerizable or gellable polymers and/or monomers; a target; polymerizing or gelling the polymers and/or monomers in each of the plurality of vessels to form a hardened particle, thereby forming a plurality of hardened particles; generating a barcode sequence in each of the plurality of hardened particles, thereby generating a barcoded target sequence.
[276] The method of paragraph [275], wherein each of the plurality of vessels further comprises a single cell, a single complex of cells, or a single exosome.
[277] The method of paragraph [275] or [276], wherein the target is from the single cell, the single complex of cells, or the single exosome.
[278] The method of any one of paragraphs [275]-[277], wherein the target is a target polynucleotide, a target polypeptide, or a target organelle.
[279] The method of paragraph [278], wherein the target polynucleotide is a target ribonucleic acid (RNA) or a target deoxyribonucleic acid (DNA).
[280] The method of paragraph [278], wherein the target RNA is total RNA, a messenger RNA, a non-coding RNA, a transfer RNA, a ribosomal RNA, a short interfering RNA, a short-hairpin RNA, a micro-RNA, small nucleolar RNA, a Piwi-interacting RNA, a tRNA-derived small RNA, a small rDNA-derived RNA, a circular RNA, or any combination thereof.
[281] The method of paragraph [278], wherein the target DNA is a genomic DNA, a modified DNA, or a combination thereof.
[282] The method of paragraph [281], wherein the modified DNA is a methylated DNA.
[283] The method of paragraph [278], wherein the target organelle comprises acrosome, autophagosome, centriole, cilium, cnidocyst, eyespot apparatus, glycosome, glyoxysome, hydrogenosome, lysosome, melanosome, mitosome, myofibril, nucleolus, parenthesome, peroxisome, proteasome, ribosome (80S), stress granule, vesicle, chloroplast, endoplasmic reticulum, flagellum, Golgi apparatus, mitochondrion, nucleus, vacuole, or any combination thereof.
[284] The method of paragraph [278], wherein the target polypeptide is a peptide, a protein or a portion thereof, a modified protein or a portion thereof, a protein complex, or any combination thereof.
[285] The method of any one of paragraphs [275]-[284], wherein each of the plurality of vessels further comprises an immobilizable probe linked to one or more polymerizable or gellable polymers or monomers of the plurality.
[286] The method of paragraph [285], wherein the immobilizable probe comprises a targeting moiety.
[287] The method of paragraph [286], wherein the targeting moiety is a polynucleotide, a polypeptide, or a chemical group.
[288] The method of paragraph [287], wherein the polynucleotide is a primer or an oligonucleotide aptamer.
[289] The method of paragraph [288], wherein the primer is a reverse transcription primer.
[290] The method of paragraph [289], wherein the reverse transcription primer comprises a poly-deoxy-thymidine nucleotide sequence.
[291] The method of any one of paragraphs [288]-[290], further comprising annealing the primer to the target polynucleotide.
[292] The method of any one of paragraphs [288]-[291], further comprising extending the primer to generate a complementary strand of the target polynucleotide.
[293] The method of paragraph [292], wherein the extending is performed in the vessel or the hardened particle.
[294] The method of paragraph [287], wherein the polypeptide is an antibody or a fragment thereof, or a peptide aptamer.
[295] The method of paragraph [287], wherein the chemical group is a reactive group.
[296] The method of paragraph [295], wherein the reactive group forms a covalent bond with an amino acid side chain or a nucleobase of the target.
[297] The method of paragraph [295] or [296], wherein the reactive group is an N-hydroxysuccinimide (NHS) ester, a maleimide group, a hydrazide group, an azide group, or Label-IT linker and reactive group.
[298] The method of paragraph [296] or [297], wherein the amino acid side chain is lysine or cysteine side chain.
[299] The method of any one of paragraphs [296]-[298], wherein the amino acid side chain comprises an amine group, a carboxylate group, a carbonyl group, or a sulfhydryl group.
[300] The method of any one of paragraphs [296]-[299], wherein the nucleobase is guanine, adenine, cytosine or thymine.
[301] The method of any one of paragraphs [285]-[300], wherein the immobilizable probe comprises an immobilization moiety.
[302] The method of paragraph [301], wherein the immobilization moiety links the immobilizable probe to one or more of the polymers and/or monomers of the plurality.
[303] The method of paragraph [302], wherein the immobilization moiety a chemical that is co-polymerized with the polymers and/or monomers, a chemical or protein that stably interact with the polymers and/or monomers directly, or a chemical or protein that stably interacts with the polymers and/or monomers indirectly.
[304] The method of any one of paragraphs [301]-[303], wherein the immobilization moiety is an NHS ester, a biotin, a maleimide group, a thiol group, an azide group, an avidin or streptavidin, a single-stranded polynucleotide, a biotin group, or a methacryl group.
[305] The method of any one of paragraphs [276]-[304], further comprising contacting the plurality of vessels or hardened particles to a lysing agent.
[306] The method of paragraph [305], wherein the lysing agent comprises IGEPAL CA-630, Sarkosyl, protease K, lyticase, glusulase and zymolyase, or any combination thereof.
[307] The method of any one of paragraphs [275]-[306], wherein the barcode sequence diffuses into the plurality of hardened particles.
[308] The method of any one of paragraphs [275]-[307], wherein the vessel or the hardened particle further comprises a barcode-accepting polynucleotide.
[309] The method of paragraph [308], wherein the barcode is attached to the barcode-accepting polynucleotide.
[310] The method of paragraph [308] or [309], wherein the barcode-accepting polynucleotide is the target, the complementary strand of the target, or the immobilizable probe.
[311] The method any one of paragraphs [275]-[310], wherein the vessel or the hardened particle further comprises a detection probe.
[312] The method of paragraph [311], wherein the barcode-accepting probe is attached to the detection probe.
[313] The method of any one of paragraphs [275]-[312], wherein generating the barcode sequence comprises: pooling the plurality of hardened particle; dividing the plurality of hardened particle into separate first populations; attaching a first oligonucleotide comprising a first barcode sequence segment to the separate first populations, wherein each separate first population comprises a different first barcode sequence segment; pooling the separate first populations to provide a first pooled population; separating the first pooled population into a plurality of second populations; attaching a second oligonucleotide comprising a second barcode sequence segment to the first oligonucleotide already attached to the second populations, wherein each of the plurality of second populations comprises a different second barcode sequence segment; and pooling the plurality of second populations to provide a second pooled population comprising a barcode library.
[314] The method of paragraph [313], wherein the first barcode sequence segment or the second barcode sequence segment comprises at least 1 nucleotide in length.
[315] The method of paragraph [313], wherein the first barcode sequence segment or the second barcode sequence segment comprises at least 4 nucleotides in length.
[316] The method of paragraph [313], wherein the first barcode sequence segment or the second barcode sequence segment comprises from about 4 to about 20 nucleotides in length.
[317] The method of any one of paragraphs [275]-[316], wherein the barcode sequence is attached to the immobilizable probe, the target, or the complementary strand of the target polynucleotide.
[318] The method of any one of paragraphs [275]-[317], further comprising washing the hardened particles extensively.
[319] The method of any one of paragraphs [276]-[318], wherein the single complex of cells comprises a first cell and a second cell.
[320] The method of paragraph [319], wherein the first cell is a mammalian cell.
[321] The method of paragraph [320], wherein the mammalian cell expresses a T-cell receptor or a portion thereof.
[322] The method of any one of paragraphs [319]-[321], wherein the first cell is an immune cell.
[323] The method of paragraph [322], wherein the immune cell is a T cell.
[324] The method of any one of paragraphs [319]-[323], wherein the second cell is an antigen presenting cell.
[325] The method of any one of paragraphs [319]-[324], wherein the second cell is a yeast cell.
[326] The method of paragraph [325], wherein the yeast cell expresses a MHC molecule on its surface.
[327] The method of paragraph [326], wherein the MHC molecule is a class I MHC or a class II MHC.
[328] The method of paragraph [326] or [327], wherein the MHC molecule is expressed from a gene selected from HLA-A, HLA-B, HLA-C, HLA-DPA1, HLA-DPB1, HLA-DQA1, HLA-DQB1, HLA-DRA, HLA-DRB1, or any combination thereof.
[329] The method of any one of paragraphs [326]-[328], wherein the MHC molecule further comprises a peptide.
[330] The method of any one of the paragraphs [275]-[329], further comprising sequencing the barcoded target sequence.
[331] The method of any one of paragraphs [275]-[330], wherein the vessel is a water-in-oil droplet.
[332] The method of any one of paragraphs [275]-[331], wherein the hardened particle is a hydrogel particle.

### EXAMPLES

### Example 1: Stably Embedding cDNAs from Single Cells in Hydrogel Particles

A. Methods to Stably and Accessibly Embed cDNA in Hydrogel Particle

1. Encapsulation of Single Cells with Hydrogel Precursor, Cell Lysing Agent, and Immobilizable Probe in Water-in-Oil Droplets

A microfluidic device such as that shown in FIG. 2 can be used to mix a cell suspension and a Cell Lysing and mRNA Embedding Buffer, and partition the mixture into uniform-sized droplets. The cell suspension and the Cell Lysing and mRNA Embedding Buffer may be infused from incoming ports 201 and 202, respectively, at the same rate, causing 1:1 mixture of the fluids in the droplets. The carrier oil (such as RAN Biotechnologies, cat. no. 008-FluoroSurfactant-2wtH) can be infused from incoming port 203. Droplets or hydrogel particles can be collected through port 204. The flow rates of the three incoming fluids can be tuned so that the size of the droplets can be 0.5 to 5 nL and have a variation in size below 20%. The exact volume may depend on the cell type and the optimal volume can be determined empirically. The widths of different channels can also be tuned to optimize encapsulation efficiency.

The microfluidic device can be made of PDMS and manufactured using standard microfabrication techniques. Before using, the microfluidic device should be silanized for example using Aqupel.

The cell suspension should contain cells at sufficiently low density so that having two cells in the same droplet is unlikely, and may comprise density-matching agent (such as OptiPrep) to prevent cell sedimentation.

The Cell Lysing and mRNA Embedding Buffer can comprise (a) a cell lysing agent, (b) an immobilizable probe, (c) acrylamide, (d) bisacrylamide, and (e) a polymerization initiator. The cell lysing agent can be IGEPAL CA-630 at a final concentration (in the droplet) of 0.25%. The immobilizable probe can be an Acrydite-modified polyT-based RT primer having the following sequence: /5Acryd/CGACATCTAA CCTAGCTC TTTTTTTTTT TTTTTTTTTT TTTTTTTTTTV (SEQ ID NO: 3), where/5Acryd/is the 5′ Acrydite modification. The first 18 nt (‘CGACATCTAA CCTAGCTC’ (SEQ ID NO: 4)) provides a primer binding site for downstream processing. This domain can be called Domain A (or DA) throughout this disclosure. The polymerization initiator can be the photoinitiator lithium phenyl-2,4,6-trimethylbenzoylphosphinate (LAP) which can be synthesized, such as by the methods described in by Fairbanks et al., Biomaterials 30(2009):6702 and Majima and Schnabel, Makromol Chem 192(1991):2307, and can be activated by a 365-nm LED UV source. 2. Optimization of gelling condition

Illumination with 365 nm UV (e.g., using an LED light source) can be used to trigger the gelling. The gelling condition (i.e., concentration of the photoinitiator, intensity and duration of the illumination) can be easily tuned. An acceptable gelling condition should yield high structural integrity and high immobilization efficiency of the immobilizable probe.

High structural integrity can be considered achieved if aqueous suspension of separable, sphere-shaped hydrogel particles can be obtained after emulsion breaking.

To test immobilization efficiency of the immobilizable probe, one may use a surrogate immobilizable probe with the following sequence: 5′-/5Acryd/TTTTTTTTT/deoxyU/TTTTTTTTT/deoxyU/TTTTTT/deoxyU/TTT/3-FAM/-3′ (SEQ ID NO: 5), where deoxyU is deoxyuridine nucleotide and 3-FAM is 3′ fluoroscein modification. After the emulsion is broken, the hydrogel particles can be washed extensively. Then, the USER enzyme mix (e.g. from NEB) can be added to cut the deoxyU nucleotide on the surrogate immobilizable probe and release the 3′ remnant (5′-TTT/3-FAM/-3′) to the solution. After washing and collecting all supernatant, the hydrogel particles can be examined using fluorescent microscope to confirm that nearly all fluorescein has been removed, and the 3′ remnant in the supernatant can be quantified using a fluorescence plate reader with proper standard curve. The amount of the surrogate immobilizable probe can be calculated, and this quantity can be divided by the input amount of surrogate immobilizable probe to obtain the immobilization efficiency. An immobilization efficiency value of >70% should be considered acceptable. 3. Emulsion-breaking, reverse transcription, second-strand synthesis and clean up

The emulsion can be broken by adding detergent that destabilizes the water-oil interface. For example, if RAN Biotechnologies, cat. no. 008-FluoroSurfactant-2wtH is used as the carrier oil, then one may break 1 V of emulation using the following procedure. To ensure that the complex formed by mRNA and RT primer is stable and to minimize mRNA degradation, the procedure should be carried out on ice or at 4° C. whenever possible.

(a) Add 1 V of Tris-buffered saline-EDTA-Triton [TBSET, prepared by combining 822 mL of nuclease-free water, 10 mL of 1 M Tris-HCl (pH 8.0), 137 mL of 1 M NaCl, 1.35 mL of 2 M KCl, 20 mL of 0.5 M EDTA, and 10 mL of 10% (vol/vol) Triton X-100 in nuclease-free water].

(b) Add 2 V of 20% (vol/vol) PFO (prepared by combining 8 mL of HFE-7500 oil with 2 mL of 1H,1H,2H, 2H-perfluorooctanol)

(c) Centrifuge at 5,000 g for 30 s, remove the bottom PFO phase.

(d) Repeat steps (b) and (c) until the hydrogel particles appear as a transluscent (rather than milky) mass

(e) Add 2 V of 1% (vol/vol) Span-80 in hexane (prepared by combining 99 ml of hexane and 1 ml of Span-80), vortex and centrifuge at 5,000 g for 30 s, remove the top hexane layer.

(f) Repeat step (e).

After the emulsion is broken, reverse transcriptase, TSO and additives that support reverse transcription will be added to the hydrogel particles, these reagents can diffuse into the hydrogel particles and complete reverse transcription and template switching.

The hydrogel particles can be washed by adding wash buffer to the particles, incubating for 5 to 15 min, centrifugation at 5,000 g for 30 s, and removing the supernatant. Depending on the applications, a variety of wash buffer can be used. Such washing can remove unwanted enzymes, oligonucleotides (such as unused TSO, un-immobilized RT primer), buffer components that is no longer used (such as DTT and nucleotide triphosphates).

Second-strand synthesis will be performed by (a) adding RNaseH to degrade the mRNA on the mRNA:cDNA hybrid, (b) adding a second-strand synthesis primer essentially having the same sequence as the TSO, and (c) adding a DNA polymerase of reverse transcriptase which extends the second-strand synthesis primer using the first-strand cDNA as a template.

The second-strand synthesis primer has a deoxyuridine nucleotide at about the 4^{th}, the 8^{th}, and the 12^{th} nucleotide from its 5′ end. After second-strand synthesis, the hydrogel particles can be treated with the USER Enzyme Mix (New England Biolabs) to cleave the deoxyuridine nucleotides, leaving a ˜12-nt long single-stranded region at the 3′ end of the first-strand cDNA. This single-stranded region can be called 3′ barcode acceptor site, which can be useful in the tagging process.

B. Alternatives and extensions

1. Cell lysing agent

The cell lysing agent can be IGEPAL CA-630 at a final concentration (in the droplet) of 0.1% to 1%, or Sarkosyl at a final concentration (in the droplet) of 0.1% to 0.5%. Additional agents that help cell lysis may be added to the Cell Lysing and mRNA Embedding Buffer as well, such as protease K, lyticase, glusulase and zymolyase. These enzymes can be particularly useful if the cell has a cell wall (e.g., a yeast cell). Fixation reversal agent can be used to make mRNA accessible to primers and reverse transcriptase.

2. Cells within the cell suspension

Cells can be replaced with any biological particles, for example, cell complexes, cell-cell conjugates, exosomes, and organelles.

3. Target molecules and immobilizable probe

The example above shows how one may stably embed mRNA in hydrogel particles. However, a wide range of target molecules can be stably embedded in hydrogel particles by employing different types of immobilizable probes. For example, when proteins are target molecules of interest, the targeting moiety can be an antibody specific for a protein target. For another example, when polynucleotides are target molecules of interest, the targeting moiety can be an oligonucleotide which hybridizes with a target polynucleotide. The targeting moiety can be a reverse transcription primer which hybridizes with all mRNA species in a cell. The targeting moiety can be a primer having a specific or designed sequence which hybridizes with a particular DNA or RNA of interest.

4. Methods to create cell-entrapping droplets

The partition can be an emulsion formed passively using a microfluidics device. These methods can involve squeezing, dripping, jetting, tip-streaming, tip-multi-breaking, or similar. Passive microfluidic droplet generation can be modulated to control the particle number, size, and diameter by altering the competing forces of two different fluids. These forces can be capillary, viscosity, and/or inertial forces upon the mixing of two solutions.

5. Choice of polymer framework

Polyacrylamide can be a choice to be the polymer framework for the hydrogel. The polyacrylamide gel can be polymerized from monomers (e.g., acrylamide) and crosslinkers (e.g., bis-acrylamide) in the droplets which offers some advantage such as low viscosity and ease of immobilizing the immobilizable probe with high immobilization efficiency.

In addition, several polysaccharide-based polymers can be considered too, such as alginate and agarose. Alginate can be readily derivatized which offers many options to immobilize the immobilizable probe (see, e.g., Pawer and Edgar, Biomaterials 33(2012), 3279). Even when the polymer is not derivatized, one may still be able to achieve stable physical association between the immobilizable probe and the polymer framework. For example, large particles (e.g., micron-sized streptavidin-coated beads) can be entrapped in underivatized polymer framework, and the immobilizable probe can form stable physical association with the surface or interior of the large particle.

With some polymers, the gelling can be reversed. That is, the hydrogel particles can be converted from a gel state to a fluid state. In other words, the gel can be melted. This may be useful in some situations such as recovering the target molecule or DNA from the hydrogel particles. For example, the bis-acrylamide in polyacrylamide gel can be replaced with crosslinkers such as DATD (diallyl-tartardiamide), DHEBA (dihydroxyethylene-bis-acrylamide), and BAC (bis-acrylylcystamine). These crosslinkers can be cleaved by several reducing agents or oxidizing agents. Alginate gels can be easily melted by EDTA, and agarose gel can be melted by high temperature.

6. Polymerization initiator and gelling condition

When making polyacrylamide hydrogel particles in droplets, the polymerization initiator may be ammonium persulfate (APS) or a water-soluble photoinitiator.

In the situations wherein APS is used as the initiator, accelerator tetramethylethylenediamine (TEMED) may be added to the carrier oil. For example, 2.5 mL of carrier oil and 10 μL of TEMED may be combined to form TEMED-containing carrier oil. If APS and TEMED-containing carrier oil are used, then the resulting emulsion can be incubated at 65° C. for overnight to induce the polymerization of the polyacrylamide hydrogel.

Other water-soluble photoinitiators can be used. The photoinitiator can be excited with 365 nm UV. Several options other than LAP exist, such as (a) sodium 4-[2-(4-morpholino)benzoyl-2-dimethylamino]-butylbenzenesulfonate (MBS) which can be synthesized, such as by the methods described in Kojima et al., Chem Mater 10(1998):3429, and (b) molecular complex formed by 2,2-dimethoxy-2-phenyl acetophenone and methylated-O-cyclodextrin (DMPA:MβCD complex) which can be prepared, such as by the methods described in Ayub et al., Advanced Materials Research 1125(2015):84.

In the situations wherein alginate is used as the polymer framework, the gelling can be triggered by delivering calcium ions directly to the droplets in the microfluidic device, or by releasing of photocaged calcium. In the situations wherein agarose is used as the polymer framework, the gelling can be triggered by lowering the temperature.

After the biological particles are distributed to multiple partitions and the target nucleic acid has been released, the monomers and cross-linkers within the partition can be polymerized to form a cross-linked polymer network which supports the hydrogel. Several methods can be used to trigger such polymerization. One method is to use ammonium persulfate (APS) and N,N,N′,N′-tetramethylethane-1,2-diamine (TEMED). The APS can be included in the aqueous phase (e.g., in the droplets) and the TEMED can be added to the partitioning oil. After droplet generation, the emulsion can be heated (e.g., at at least 50° C., at least 55° C., at least 60° C., or at least 65° C.) for a prolonged period of time (e.g., at least 10, 12, 15, or 20 hours, or overnight), which triggers the polymerization. However, in some applications such process may create difficulties in other aspects such as maintaining the quality of the target nucleic acid. Therefore, in some cases it may be needed to trigger the polymerization faster and with a milder treatment. Long wavelength UV (e.g., >360 nm) photo-initiation can be an option. Convenient LED-based light source are available for 365 nm illumination.

A number of molecules or molecular complexes can be water-soluble photo-initiator that is compatible with long wavelength UV, for example, lithium- and magnesium phenyl-2,4,6-trimethylbenzoylphosphinates (TMPPL and TMPPM). They are effective water-soluble photo-initiators for the free-radical polymerization of appropriate monomers such as acrylamide and methacrylamide in aqueous solution. TMPPL (also called LAP) can be used to trigger the formation of biocompatible hydrogel. Similarly, sodium 4-[2-(4-morpholino)benzoyl-2-dimethylaminol-butylbenzenesulfonate (MBS), via sulfonation of 2-benzyl-2-(dimethylamino)-1-(4-morpholinophenyl)-1-butanone (BDMB), can be used as water-soluble long wavelength UV photo-initiator. Commonly used, water-insoluble photo-initiators may also be formulated in water-soluble form such as by complexing with methylated-β-cyclodextrin (MβCD). For example, 2,2-dimethoxy-2-phenyl acetophenone (DMPA) complexed with MβCD as water-soluble photo-initiator can be used.

7. Reverse transcription in the droplets

The Cell Lysing and mRNA Embedding Buffer may additionally comprise reverse transcriptase, template switching oligo (TSO) and additives that support reverse transcription (such as DTT). If (a) the Cell Lysing and mRNA Embedding Buffer comprises reverse transcriptase, TSO, and additives that support reverse transcription, and (b) the cell lysing agent in the Cell Lysing and mRNA Embedding Buffer does not inhibit the reverse transcriptase, then reverse transcription can be carried out before the emulsion is broken. The reverse transcription can also be carried out before the gelling is triggered. For example, the emulsion created by the microfluidic device can be incubated at the optimal temperature of the reverse transcriptase for 1 to 2 hours before triggering the gelling of the hydrogel.

### Example 2: Tagging the Molecules of Interest in Each Hydrogel Particle with a Unique DNA Barcode

A. Adding bead barcode to cDNA strand by split-and-pool primer extension

Barcodes can be added by split-and-pool extension. For each unique barcode, it can be added by one or more round of extension, and in each round, there are a certain number of variations of barcode-containing segments. Each segment is a short oligonucleotide which can be added to the cDNA strand.

After the double-stranded cDNA-RNA hybrid, which has a ˜12-nt 3′ barcode acceptor site at the 3′ end of the first-strand cDNA, is stably embedded in the hydrogel particle following the process described in Example 1, Section A, the cDNA molecule can be barcoded so that essentially all the cDNA molecules originated from the same cell get the same barcode, whereas cDNA molecules originated from different cells have different barcode. This can be done using a split-and-pool process.

Before doing the tagging experiment, 384 variations of the BarcodeThree A (named BarcodeThree A1 to BarcodeThree A384, where ‘Three’ denotes that the barcode can be attached a 3′ barcode acceptor site A, shown as 3A-a in FIG. 4B) will be prepared. The BarcodeThree A is made of an oligonucleotide chain comprising a ˜12-nt 3′ barcode donor site A (3BDA, shown as 3A-a* in FIGS. 4A and 4B), a 5- to 8-nt barcode region (BC-a* in FIGS. 4A and 4B), and a ˜12-nt antisense 3′ barcode acceptor site B (3BAB*, shown as 3A-b* in FIGS. 4A and 4B). Different version of BarcodeThree A has different barcode sequences and may have different lengths. The 3′ end of the BarcodeThree A may contain a modification that blocks primer extension (such as 3′ amino modification, 3′ phosphorylation, or inverted dT).

Similarly, 384 versions of BarcodeThree B will be prepared (FIG. 4A). BarcodeThree B can be designed to be similar to BarcodeThree A, except that the ˜12-nt 3′ barcode donor site A (3BDA) is replaced by a ˜12-nt 3′ barcode donor site B (3BDB, shown as 3A-b* in FIGS. 4A and 4B); the barcode domain is replaced by another set of sequence (shown as BC-b* in FIGS. 4A and 4B); and the ˜12-nt antisense 3′ barcode acceptor site B (3BAB*) is replaced by a ˜12-nt antisense 3′ barcode acceptor site C (3BAC*, shown as 3A-c* in FIGS. 4A and 4B).

B. Alternatives and extensions

It is helpful to assign DNA barcode to target molecules in a way that all target molecules from the same cell get the same barcode, whereas target molecules from different cells have different barcode.

1. More rounds of split-and-pool

Doing more-rounds of split-and-pool can result in more numbers of unique barcodes generated. Using 384 variations of barcode-containing segments per round as an example, for a particular barcode, if one round of barcoding is performed, 384 unique barcodes will be generated, and if two rounds of barcoding is performed, 384×384 unique barcodes will be generated, and if “N” rounds of barcoding is performed, 384^{N} unique barcodes will be generated.

2. Tagging at the 5′ end

As an alternative to adding the barcode to the 3′ end of the cDNA via primer extension as described in Examples 1 and 2, the barcode can be added to the 5′ end of the cDNA via ligation. For example, the immobilizable probe can be designed as shown in FIG. 4D, where “X” is the immobilization moiety, RTP is the RT primer, DA* is an arbitrary sequence attached to X, DA is complementary to DA* and can stably hybridize to DA* at experimental condition, and 5A-a is a 5′ barcode acceptor site. Barcode sequences can be ligated to the 5′ end of 5A-a in a split-and-pool fashion, similar to the strategy described in Example 2.

### Example 3: Stably Incorporating Oligonucleotide in ‘Meltable’ Polyacrylamide Hydrogel Beads and In Situ Barcode Ligation

Two types of polyacrylamide hydrogel beads were manufactured: oligonucleotide-incorporated beads and “naked” beads. For oligonucleotide-incorporated beads, an Acrydite-modified oligonucleotide (named Acrydite.DA2*, which is an example of the oligonucleotide DA* shown in FIG. 4D) was mixed with acrylamice, N,N′ bis(acryloyl)cystamine (BAC), in the presence of photo polymerization agent lithium phenyl-2,4,6-trimethylbenzoylphosphinate (TMPPL). The composition contains 10-20% Acrylamide, 1-5% BAC, 0.05-1% TMPPL, 10-50 μM Acrydite.DA2*, and 1-10 μM RT primer. The mixture was made into ˜50 μm diameter droplets using a flow-focusing microfluidics device. The droplets were exposed to 365 nm UV for 30 s which induces polymerization and turns droplets into beads. The beads were demulsified using perfluorooctanol (PFO). The “naked” beads were produced in the same manner except Acrydite.DA2* was omitted.

Next, a FAM-labeled oligonucleotide whose sequence is complementary to Acrydite.DA2* was incubated with both types of beads. It can be seen from FIG. 7 that only the oligonucleotide-incorporated beads were labeled. This shows that Acrydite.DA2* was stably attached to the polyacrylamide framework in the oligonucleotide-incorporate beads. In addition, after treatment of DTT, both types of beads can be melted, as seen in FIG. 8.

To show that RT primers can be ligated to DNA barcodes (which effectively barcode the cDNA extended from the RT primer), a new type of oligonucleotide-incorporated beads was made where the Acrydite.DA2* was replaced with a duplex formed by Acrydite.DA2* and another oligonucleotide named T14d.sN6.DA2.TRAC.FAM. The sequence of T14d.sN6.DA2.TRAC.FAM is

(SEQ ID NO: 6)
5′-ATGCTGACGTGA gctg NNNNNN cagc
CCGAGAGTGATTGCTTGTGACGCC GGTACAC
GGCAGGGTCAGGGTTCTGGATAT-3′,

where the underlined portion is complementary to Acrydite.DA2*, the lower-case portion forms a hairpin with a randomized sequence in the middle to serve as UMI, the 5′ end region with the sequence 5′-ATGCTGACGTGA-3′ (SEQ ID NO: 7) serves as the barcode acceptor site, the 3′ end region with the sequence 5′-GGTACACGGCAGGGTCAGGGTTCTGGATAT-3′ (SEQ ID NO: 8) may serve as RT primer which targets a target of interest (e.g., human TCR alpha chain constant region (TRAC)). The 5′ end of T14d.sN6.DA2.TRAC.FAM is phosphorylated to facilitate ligation. For easy tracking of ligation, the 3′ end of T14d.sN6.DA2.TRAC.FAM was also FAM-labeled. In some applications, the 3′ FAM modification (which blocks primer extension) will be omitted.

Next, a barcode-carrying oligonucleotide named T14i.rcbc1.1.U.T2d.bc1.1 was diffused into the beads along with a DNA ligase so that T14i.rcbc1.1.U.T2d.bc1.1 can be ligated to the barcode acceptor site (FIG. 9A). T14i.rcbc1.1.U.T2d.bc1.1 has the following sequence

(SEQ ID NO: 9)
5′-TCACGTCAGCATCTATCTGTTuATCTTGTTGTGTCGCGA
TaAACAGATAG-3′.

The 5′ region with the sequence 5′-TCACGTCAGCAT-3′ (SEQ ID NO: 10) is complementary to the barcode acceptor site of Acrydite.DA2*. The lower-case ‘u’ is a deoxyuridine base which can later be cleaved using the USER enzyme mix (available from New England Biolabs). The underlined regions can form a hairpin, serve as vessel barcode and can be replaced by other sequences in other variations of the barcode.

Next, the beads were treated with USER enzyme mix which can diffuse into the beads and cleave the deoxyuridine nucleotide and linearize the hairpin (FIG. 9B). The newly exposed 5′ end (a domain named T2d, carrying a 5′ phosphate) can be ligated in the next round of barcode ligation (which mimics the next round of ligation in the split-and-pool barcode ligation process).

Similar to the last round of barcode ligation, the beads were treated with a new barcode-carrying oligonucleotide named T2i.rcbc2.1.U.T3d.bc2.1, having the sequence:

(SEQ ID NO: 11)
5′-GCGACACAACAAGATCTATCTGTTuCACACGGAACCAAC
AGTGaAACAGATAG-3′,

and a DNA ligase (FIG. 9C). Again, the underlined regions can torm a hairpin and serve as vessel barcode. The beads were then treated with USER enzyme mix to cleave the deoxyuridine nucleotide exposing a new 5′ domain (named T3d) for further barcode ligation (FIG. 9D). The beads were subject to another round of barcode ligation of barcode ligation and USER treatment (FIG. 9E).

After each round of barcode ligation, an aliquot of the beads was saved, melted with DTT, and subject to denaturing PAGE. The gel image (FIG. 10) shows that the barcodes were successfully ligated to the barcode acceptor.

### Example 4: Template-Switched cDNA from a Single Cell can be Stably Entrapped in Agarose Gel Using Linear Polymer as Diffusion Restricting Agent

Two reverse transcription primers targeting the human TRAC and TRBC regions (i.e., constant domains of TCR alpha and beta chain), respectively, were covalently linked to linear polyacrylamide. To do this, amine-modified linear polyacrylamide was first prepared by copolymerizing acrylamide and N-(3-Aminopropyl)methacrylamide hydrochloride, and then reacted with NHS-azide and DBCO-modified primer.

Then, a suspension comprising 0.5-3% low-melting temperature agarose, Jurkat cells (at 1,000 cells/uL), 1×SuperScript IV buffer, 5 mM DTT, 0.5 mM dNTP (each), 1 μM TSO (sequence: AAGCAGTGGTATCAACGCAGAGTACATrGrG+G (SEQ ID NO: 12), where ‘rG’ denote ribose G and ‘+G’ denotes LNA G), 2 U/μL RNaseOUT, 10 U/uL SuperScript IV enzyme were prepared at a warm temperature to keep the agarose from gelling. The cell suspension was encapsulated in droplets using a flow-focusing chip such as the “hydrogel bead generation device” described in Zilionis et al., 2017 (PMID 27929523) and droplet oil (e.g., FluoroSurfactant in HFE7500 available from RAN Biotechnologies or equivalent).

The emulsions were incubated at 50° C. for 30 to 90 min which lyses the cells and allows reverse transcription to occur. Optionally, a nucleic-acid staining dye (e.g., SYBR Gold) can be included in the cell suspension which stains the primers, TSO, as well as the nucleic of lysed cells. Such staining can help examination of the emulsion using fluorescence microscope to assess size distribution cell density.

After the 50° C. incubation, the emulsions were demulsified using PFO, and the beads were washed with copious volume of PBS or water repeatedly for a total of −2 h. Next, two PCR reactions was set up each using 2 μL of beads as input. The first PCR contained a forward primer (having the sequence: 5′-GCAGTGGTATCAACGCAGAGTAC-3′ (SEQ ID NO: 13)) that targets the TSO sequence and a reverse primer (having the sequence 5′-gctggtacacggcagggtc-3′ (SEQ ID NO: 14)) targeting TRAC. The second PCR contained the same forward primer as the first PCR and a reverse primer (having the sequence 5′-tctgcttctgatggctcaaacacA-3′ (SEQ ID NO: 15)) targeting TRBC. The first and second PCR amplifies template-switched TCR alpha chain and TCR beta chain, respectively, and the products are shown in Lanes 2 and 4 of FIG. 11, respectively. Lane 1 and 3 showed negative-control PCR of the first and second PCR reaction, respectively where no bead was provided. It can be seen that these template-switched cDNA can be readily detected in washed beads, which indicates that the template-switched cDNA was stably entrapped in the gel beads.

What is claimed is:

1.-116. (canceled)

117. A method of barcoding comprising
(a) contacting a barcode to a hardened particle, wherein the hardened particle comprises:
(i) polymerized or gelled polymers and/or monomers, and
(ii) a target or derivative thereof from a single cell;
(b) diffusing the barcode into the hardened particle; and
(c) attaching the barcode to the target or derivative thereof.

118. The method of claim 117, further comprising forming a compartment comprising the hardened particle prior to contacting the barcode.

119. The method of claim 118, wherein forming the compartment comprising the hardened particle comprises polymerizing or gelling a plurality of polymerizable or gellable polymers and/or monomers within the compartment, thereby forming the hardened particle.

120. The method of claim 119, wherein the compartment comprises the single cell comprising the target and an immobilizable probe.

121. The method of claim 120, wherein the target or derivative thereof is bound to the immobilizable probe.

122. The method of claim 120, wherein the immobilizable probe comprises a targeting moiety that links the immobilizable probe to the target or derivative thereof.

123. The method of claim 122, wherein the targeting moiety is a polynucleotide, a polypeptide, or a chemical group.

124. The method of claim 123, wherein the target is a target polynucleotide, and wherein the targeting moiety is a primer.

125. The method of claim 124, further comprising extending the primer to generate a complementary strand of the target polynucleotide prior to attaching the barcode to the target or derivative thereof.

126. The method of claim 123, wherein the target is a target polypeptide, and wherein the targeting moiety is an antibody or a fragment thereof, or a peptide aptamer.

127. The method of claim 123, wherein the targeting moiety is a chemical group that forms a covalent bond with an amino acid side chain or a nucleobase of the target.

128. The method of claim 120, wherein the immobilizable probe comprises an immobilization moiety that links the immobilizable probe to the polymerized or gelled polymers and/or monomers of the hardened particle.

129. The method of claim 128, wherein the immobilization moiety is a chemical that is co-polymerized with the polymerized or gelled polymers and/or monomers, a chemical or protein that stably interacts with the polymerized or gelled polymers and/or monomers directly, or a chemical or protein that stably interacts with the polymerized or gelled polymers and/or monomers indirectly.

130. The method of claim 120, wherein attaching the barcode comprises attaching the barcode to a barcode-accepting polynucleotide, wherein the barcode-accepting polynucleotide is the immobilizable probe or the target or derivative thereof.

131. The method of claim 117, wherein the hardened particle further comprises a detection probe that binds to the target or derivative thereof.

132. The method of claim 131, wherein the method comprises attaching the barcode to the detection probe.

133. The method of claim 117, wherein the hardened particle is a plurality of hardened particles.

134. The method of claim 133, wherein the method comprises:
(a) pooling the plurality of hardened particles;
(b) dividing the plurality of hardened particles into separate first populations of hardened particles;
(c) attaching a first oligonucleotide comprising a first barcode sequence segment to the separate first populations of hardened particles, wherein each separate first population of hardened particles comprises a different first barcode sequence segment;
(d) pooling the separate first populations of hardened particles to provide a first pooled population of hardened particles;
(e) separating the first pooled population of hardened particles into a plurality of second populations of hardened particles;
(f) attaching a second oligonucleotide comprising a second barcode sequence segment to the first oligonucleotide attached to the plurality of second populations of hardened particles, wherein each second population of the plurality of second populations of hardened particles comprises a different second barcode sequence segment; and
(g) pooling the plurality of second populations of hardened particles to provide a second pooled population of hardened particles.

135. The method of claim 117, further comprising washing the hardened particle.

136. A composition comprising a plurality of vessels, each vessel of the plurality comprising
(a) a hydrogel particle;
(b) an immobilizable probe embedded within the hydrogel particle, wherein the immobilizable probe is immobilized to a diffusion restricting agent; and
(c) a target or derivative thereof embedded within the hydrogel particle and linked to the immobilizable probe, wherein the target is from a single cell, a single complex of cells, or a single exosome, or the derivative thereof is a derivative of a target from a single cell, a single complex of cells, or a single exosome;
wherein the hydrogel particle has a volume that is at least 50% of a volume of the vessel.


SEQUENCE LISTING (16 sequences)

SEQ ID NO: 1 - DNA, 31 bp, Artificial Sequence
  Description of Artificial Sequence Synthetic primer
  misc_feature (1)..(30): This region may encompass 20-30 nucleotides
  misc_feature (31)..(31): May or may not be present
ttttttttttttttttttttttttttttttv

SEQ ID NO: 2 - protein, 16 aa, Artificial Sequence
  Description of Artificial Sequence Synthetic peptide
AEAEAKAKAEAEAKAK

SEQ ID NO: 3 - DNA, 49 bp, Artificial Sequence
  Description of Artificial Sequence Synthetic primer
cgacatctaacctagctcttttttttttttttttttttttttttttttv

SEQ ID NO: 4 - DNA, 18 bp, Artificial Sequence
  Description of Artificial Sequence Synthetic primer
cgacatctaacctagctc

SEQ ID NO: 5 - DNA, 30 bp, Artificial Sequence
  Description of Artificial Sequence Synthetic oligonucleotide
  Description of Combined DNA/RNA Molecule Synthetic oligonucleotide
tttttttttutttttttttuttttttuttt

SEQ ID NO: 6 - DNA, 80 bp, Artificial Sequence
  Description of Artificial Sequence Synthetic oligonucleotide
  modified_base (17)..(22): a, c, t, g, unknown or other
atgctgacgtgagctgnnnnnncagcccgagagtgattgcttgtgacgccggtacacggcagggtcagggttctggatat

SEQ ID NO: 7 - DNA, 12 bp, Artificial Sequence
  Description of Artificial Sequence Synthetic oligonucleotide
atgctgacgtga

SEQ ID NO: 8 - DNA, 30 bp, Artificial Sequence
  Description of Artificial Sequence Synthetic oligonucleotide
ggtacacggcagggtcagggttctggatat

SEQ ID NO: 9 - DNA, 50 bp, Artificial Sequence
  Description of Artificial Sequence Synthetic oligonucleotide
  Description of Combined DNA/RNA Molecule Synthetic oligonucleotide
tcacgtcagcatctatctgttuatcttgttgtgtcgcgataaacagatag

SEQ ID NO: 10 - DNA, 12 bp, Artificial Sequence
  Description of Artificial Sequence Synthetic oligonucleotide
tcacgtcagcat

SEQ ID NO: 11 - DNA, 53 bp, Artificial Sequence
  Description of Artificial Sequence Synthetic oligonucleotide
  Description of Combined DNA/RNA Molecule Synthetic oligonucleotide
gcgacacaacaagatctatctgttucacacggaaccaacagtgaaacagatag

SEQ ID NO: 12 - DNA, 30 bp, Artificial Sequence
  Description of Artificial Sequence Synthetic oligonucleotide
aagcagtggtatcaacgcagagtacatggg

SEQ ID NO: 13 - DNA, 23 bp, Artificial Sequence
  Description of Artificial Sequence Synthetic primer
gcagtggtatcaacgcagagtac

SEQ ID NO: 14 - DNA, 19 bp, Artificial Sequence
  Description of Artificial Sequence Synthetic primer
gctggtacacggcagggtc

SEQ ID NO: 15 - DNA, 24 bp, Artificial Sequence
  Description of Artificial Sequence Synthetic primer
tctgcttctgatggctcaaacaca

SEQ ID NO: 16 - DNA, 20 bp, Artificial Sequence
  Description of Artificial Sequence Synthetic oligonucleotide
tttttttttttttttttttt